(19) United States
(12) Patent Application Publication
(10) Pub. No.: US 2002/0128213 A1
(43) Pub. Date: Sep. 12, 2002

(54) Sixteen-membered macrolide compounds

(76) Inventors: Leonard Katz, Oakland, CA (US); Gary Ashley, Alameda, CA (US); Mark A. Burlingame, San Francisco, CA (US); Steven D. Dong, San Francisco, CA (US); Hong Fu, Union City, CA (US); Yong Li, Palo Alto, CA (US); David C. Myles, Kensington, CA (US)

Correspondence Address: Brenda J. Wallach, Ph.D., Morrison & Foerster LLP, Suite 500, 3811 Valley Centre Drive, San Diego, CA, 92130-2332, US

(21) Appl. No.: 09/969,177

(22) Filed: Sep. 24, 2001

Related U.S. Application Data
(60) Provisional application No. 60/251,338, filed on Dec. 4, 2000.
(60) Provisional application No. 60/269,693, filed on Feb. 17, 2001.
(60) Provisional application No. 60/234,994, filed on Sep. 25, 2000.

(51) Int. Cl. A61K 31/7048; C07H 17/08

(52) U.S. Cl. 514/30; 536/7.1

(57) ABSTRACT

The present invention provides novel sixteen-membered macrolide compounds that are useful as anti-infective agents or as intermediates thereto. The present invention also provides methods for the preparation of these compounds, and methods and formulations for their use. In one aspect of the present invention, sixteen-membered macrolide possessing a side chain Z are provided where Z is aliphatic, aryl, alkylaryl, halide, ═NOR³, ═NNHR³, or —W—R³ where W is O, S, NC(═O)R⁴, NC(═O)OR⁴, NC(═O)NHR⁴ or NR⁴ where R³ and R⁴ are each independently hydrogen, aliphatic, aryl or alkylaryl. In another aspect of the present invention, bicyclic compounds are provided where one of the cyclic-components is a sixteen-membered macrolide and the other is a cyclic moiety whose cyclic structure is formed by between 3 and 10 atoms. In another aspect of the present invention, sixteen-membered macrolide compounds that bind to the domain II region of the 23S RNA are provided.

This application asserts priority to U.S. Provisional Application No. 60/269,693 filed Feb. 17, 2001 entitled NOVEL SIXTEEN-MEMBERED MACROLIDES by inventors Leonard Katz and Gary Ashley; U.S. Provisional Application No. 60/251,338 filed Dec. 4, 2000 by inventors Leonard Katz and Gary Ashley; and U.S. Provisional Application No. 60/234,994 filed Sep. 25, 2000 entitled NOVEL MACROLIDES by inventors Leonard Katz and Gary Ashley, all of which are incorporated herein by reference.

### BACKGROUND

Sixteen-membered macrolides as potentially new anti-infective agents have not been fully explored. Due to the increasing incidence of antibiotic resistance, novel compounds possessing antibiotic activity are both needed and desired.

In general, macrolide antibiotics bind to sites in the ribosome complex that disrupt protein synthesis in target organisms by inhibiting one or more processes in the growth of the peptide chain. Structure-activity relationship (“SAR”) studies of macrolide antibiotics have identified three prokaryotic ribosomal binding regions that are associated with antibacterial activity. All three involve the 23S RNA. The first two sites are located in domain V of the RNA and are referred to as the A2058 region (so named because adenosine is the base at the 2058 position in E. coli) and the peptidyl transferase region respectively. The third site is located in domain II of the 23S RNA.

The A2058 region is important to the activity of most 14-membered and sixteen-membered macrolide antibiotics. Consequently, modifications at this region that disrupt or otherwise interfere with macrolide binding are among the strategies used by resistant strains. For example, methylation of A2058 decreases the binding affinities of 14-membered erythromycin-like antibiotics so that these compounds are no longer effective at inhibiting the translation process.

Some naturally occurring sixteen-membered macrolides and derivatives are effective against resistant strains by having a side chain that binds to the peptidyl transferase region and/or inhibits peptidyl transferase activity. Illustrative examples of such compounds are carbomycin B (where R is isovaleryl) and various 4″-acyl derivatives of demycinosyl-tylosin (where R is acyl):

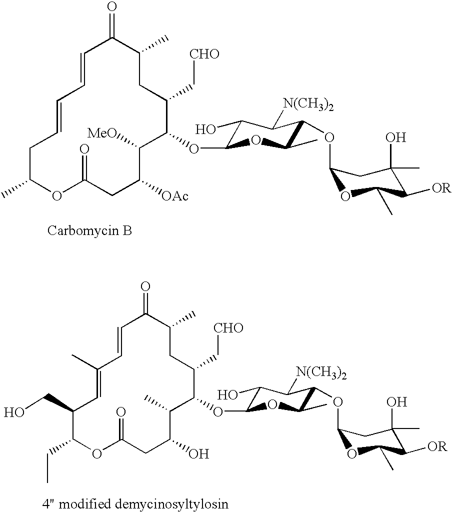

Another strategy for combating resistance is to compensate for the loss of binding affinity at the A2058 region by having one or more side chains that bind to another region of the ribosomal complex such as the domain II region. For example, ketolides that bind to the A2058 region and to the domain II region have been found to be effective against some resistant strains. Ketolides are so named due to the presence of a keto group at C-3 instead of the sugar cladinose that is found in erythromycin A.

Illustrative examples of two different families of ketolides are HMR-3647 and ABT-773. The structure of HMR-3647 is:

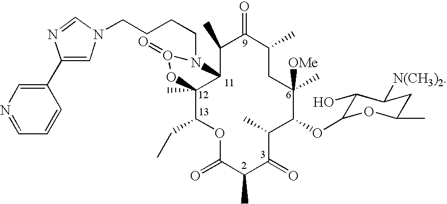

ABT-773 is similar in structure to HMR-3647 in that it is also is a cyclic carbamate-containing ketolide:

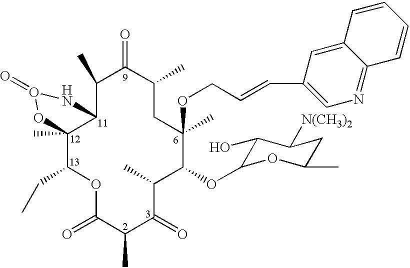

However, unlike HMR-3647, the alkylaryl moiety is off the C-6 hydroxyl and not off the carbamate nitrogen. SAR studies of these types of compounds indicate that alkylaryl groups off both the carbamate nitrogen and C-6 hydroxyl bind to the domain II region of the ribosomal complex. The affinity of the alkylaryl groups for the domain II region appears to compensate for the loss of affinity of the macrolide ring at the A2058 region in resistant strains so that these ketolide compounds bind sufficiently tightly to the ribosomal complex to disrupt the translation process.

The present invention provides novel sixteen-membered macrolides. In one aspect of the present invention, sixteen-membered macrolide compounds are provided that possess improved binding affinities to the ribosomal complex. In another aspect of the present invention, sixteen-membered macrolide compounds are provided that bind to the domain II region of the 23S RNA. In yet another aspect of the present invention, sixteen-membered macrolides are provided that bind to the domain II region of the 23 S RNA and that inhibit the peptidyl transferase activity of the 23 S RNA.

### SUMMARY

The present invention provides novel sixteen-membered macrolide compounds that are useful as anti-infective agents or as intermediates thereto. The present invention also provides methods for the preparation of these compounds, and methods and formulations for their use.

In one aspect of the present invention, sixteen-membered macrolide possessing a side chain Z are provided where Z is aliphatic, aryl, alkylaryl, halide, ═NOR³, ═NNHR³, or —W—R³ where W is O, S, NC(═O)R⁴, NC(═O)OR⁴, NC(═O)NHR⁴ or NR⁴ where R³ and R⁴ are each independently hydrogen, aliphatic, aryl or alkylaryl. in another embodiment, Z is attached to C-15 of the macrolide. In one embodiment Z is attached to C-7, C-11, or C-13 of the macrolide. In another embodiment Z is attached to C-8 of the macrolide. In another embodiment, Z is attached to C-6 of the macrolide. In another embodiment Z is attached to a substituent that is attached to C-6 to C-14. In another embodiment, Z is attached to C-12 of the macrolide. In yet another embodiment, Z is attached to C-3 or C-9 of the macrolide.

In another aspect of the present invention, bicyclic compounds are provided where one of the cyclic-components is a sixteen-membered macrolide and the other is a cyclic moiety whose cyclic structure is formed by between 3 and 10 atoms. In one embodiment, the cyclic moiety is attached at non-adjacent carbon atoms of the macrolide. In another embodiment, the cyclic moiety is attached at non-adjacent carbon atoms and is attached to the macrolide in the syn-configuration (relative to the macrolide). In another embodiment, the cyclic moiety is attached to the macrolide at C-9 and at C-11. In another embodiment, the cyclic moiety is attached to the macrolide at C-11 and at C-13.

In yet another embodiment, the cyclic moiety is attached at adjacent carbon atoms of the macrolide. In one embodiment, the cyclic moiety is attached to the macrolide at C-13 and C-14. In another embodiment, the cyclic moiety is attached to the macrolide at C-11 and C-12. In another embodiment, the cyclic moiety is attached to the macrolide at C-10 and C-11. In another embodiment, the cyclic moiety is attached to the macrolide at C-9 and C-10. In another embodiment, the cyclic moiety is attached to the macrolide at C-6 and C-7. In another embodiment, the cyclic moiety is of the formula

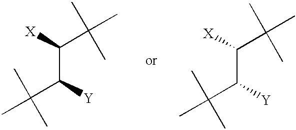

where X and Y together form the cyclic moiety that is attached to the macrolide in the syn-configuration.

In another aspect of the present invention, sixteen-membered macrolides are provided that possesses a side chain Z and that bind to the domain II region of the 23 S RNA wherein Z is as previously defined. In one embodiment, the macrolide also inhibits the peptidyl transferase activity of the 23 RNA. In another embodiment, the macrolide also possess a side chain Z′ where Z′ is an aryl- or a saccharide-containing aliphatic. In yet another embodiment, Z′ is a disaccharide, preferably a 4-mycarosyl-mycaminose wherein one or more hydroxyls of the mycarose has been converted into an aliphatic or aryl containing moiety.

In another aspect of the present invention, bicyclic compounds are provided wherein one of the cyclic components is a sixteen-membered macrolide and the other is a cyclic moiety whose cyclic structure is formed by between 3 and 10 atoms and that bind to the domain II region of the 23 S RNA, wherein the cyclic moiety is as previously defined. In one embodiment, the compound also inhibits the peptidyl transferase activity of the 23 RNA. In another embodiment, the compound also possess a side chain Z′ where Z′ is an aryl- or a saccharide-containing aliphatic. In yet another embodiment, Z′ is a disaccharide, preferably a 4-mycarosyl-mycaminose wherein one or more hydroxyls of the mycarose has been converted into an aliphatic or aryl containing moiety.

In another aspect of the present invention, recombinant DNA compounds that encode the proteins required to produce sixteen-membered macrolides as well as proteins that further modify these macrolides are provided. In one embodiment, recombinant DNA compounds that encode portions of these proteins are provided. In another aspect of the present invention, recombinant DNA compounds that encode a hybrid protein that is the product of one or more PKS genes are provided wherein the hybrid protein encodes all or portion of a protein involved in the biosynthesis of sixteen-membered macrolide. In one embodiment, the recombinant DNA compounds of the invention are recombinant DNA cloning vectors that facilitate manipulation of the coding sequences or recombinant DNA expression vectors that code for the expression of one or more of the proteins of the invention in recombinant host cells. In another aspect of the present invention, recombinant host cells are provided for the expression of PKS genes.

In another aspect of the present invention, chemobiosynthetic methods, synthetic thioesters and intermediates thereto, for making modified sixteen-membered macrolides are provided. In one embodiment, a synthetic thioester is an N-acyl-cysteamine thioester of the formula

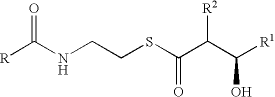

where R, R¹ and R² are each independently hydrogen, aliphatic, aryl or alkylaryl. When a thioester of the above-formula is added to a PKS that can accept such compounds as a starter unit, the PKS makes a modified lactone having the following fragment

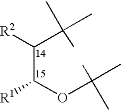

where R² and R¹ replaces the groups that are normally present at C-14 and C-15.

### DETAILED DESCRIPTION OF THE PREFERRED EMBODIMENTS

The present invention relates to novel sixteen-membered macrolides. The definitions of certain terms are defined below. These definitions apply to these terms as they are used throughout this specification, unless otherwise limited explicitly or implicitly in specific instances, either individually or as part of a larger group.

Where stereochemistry is not explicitly or implicitly specified, all stereoisomers of the inventive compounds are included within the scope of the invention, as pure compounds as well as mixtures thereof. Crystalline forms for the compounds may exist as polymorphs and as such are included in the present invention. In addition, some of the compounds may form solvates with water (i.e., hydrates) or common organic solvents, and such solvates are also encompassed within the scope of this invention.

Certain compounds of the inventions are intermediates and are often used in their protected form during chemical synthesis. Both protected and unprotected forms of these compounds are included within the scope of the present invention. A variety of protecting groups are disclosed, for example, in T. H. Greene and P. G. M. Wuts, Protective Groups in Organic Synthesis, Third Edition, John Wiley & Sons, New York (1999), which is incorporated herein by reference in its entirety. For example, a hydroxy protected form of the inventive compounds are those where at least one of the hydroxyl groups is protected by a hydroxy protecting group. Illustrative hydroxyl protecting groups include but not limited to tetrahydropyranyl; benzyl; methylthiomethyl; ethylthiomethyl; pivaloyl; phenylsulfonyl; triphenylmethyl; trisubstituted silyl such as trimethyl silyl, triethylsilyl, tributylsilyl, tri-isopropylsilyl, t-butyldimethylsilyl, tri-t-butylsilyl, methyldiphenylsilyl, ethyldiphenylsilyl, tert-butyldiphenylsilyl and the like; acyl and aroyl such as acetyl, pivaloylbenzoyl, 4-methoxybenzoyl, 4-nitrobenzoyl and aliphatic acylaryl and the like. Keto groups in the inventive compounds may similarly be protected.

The present invention includes within its scope prodrugs of certain compounds of this invention. In general, such prodrugs are functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to a subject in need thereof. Conventional procedures for the selection and preparation of suitable prodrug derivatives are described, for example, in “Design of Prodrugs”, H. Bundgaard ed., Elsevier, 1985.

As used herein, the term “aliphatic” refers to saturated and unsaturated straight chained, branched chain, cyclic, or polycyclic hydrocarbons that may be optionally substituted at one or more positions. Illustrative examples of aliphatic groups include alkyl, alkenyl, alkynyl, cycloalkyl, cycloalkenyl, and cycloalkynyl moieties. The term “alkyl” refers to straight or branched chain saturated hydrocarbon substituent, preferably a C₁-C₁₀ and more preferably a C₁-C₅. “Alkenyl” refers to a straight or branched chain hydrocarbon substituent with at least one carbon-carbon double bond, preferably a C₂-C₁₀ and more preferably a C₂-C₅. “Alkynyl” refers to a straight or branched chain hydrocarbon substituent with at least one carbon-carbon triple bound, preferably a C₂-C₁₀ and more preferably a C₂-C₅. “Cycloalkyl”, “cycloalkenyl” and “cycloalkynyl” moieties (preferably C₃-C₈, more preferably C₅-C₆) include heterocyclo groups having one or more heteroatoms (e.g., S, N, and O).

The term “aryl” refers to monocyclic or polycyclic groups having at least one aromatic ring structure that optionally include one or more heteroatoms and preferably include three to fourteen carbon atoms. Aryl substituents may optionally be substituted at one or more positions. Illustrative examples of aryl groups include but are not limited to: furanyl, imidazolyl, indanyl, indenyl, indolyl, isooxazolyl, isoquinolinyl, naphthyl, oxazolyl, oxadiazolyl, phenyl, pyrazinyl, pyridyl, pyrimidinyl, pyrrolyl, pyrazolyl, quinolyl, quinoxalyl, tetrahydronaphththyl, tetrazolyl, thiazolyl, thienyl, and the like.

The aliphatic (i.e., alkyl, alkenyl, etc.) and aryl moieties may be optionally substituted with one or more substituents, preferably from one to five substituents, more preferably from one to three substituents, and most preferably from one to two substituents. The definition of any substituent or variable at a particular location in a molecule is independent of the definition of the same substituent or variable at a different location. It is understood that substituents and substitution patterns on the compounds of this invention can be selected by one of ordinary skill in the art to provide compounds that are chemically stable and that can be readily synthesized by techniques known in the art as well as those methods set forth herein. Examples of suitable substituents include but are not limited to: alkyl; alkenyl; alkynyl; aryl; halo; trifluoromethyl; trifluoromethoxy; hydroxy; alkoxy; cycloalkoxy; heterocyclooxy; oxo; alkanoyl (—C(═O)-alkyl which is also referred to as “acyl”)); aryloxy; alkanoyloxy; amino; alkylamino; arylamino; aralkylamino; cycloalkylamino; heterocycloamino; disubstituted amines in which the two amino substituents are selected from alkyl, aryl, or aralkyl; alkanoylamino; aroylamino; aralkanoylamino; substituted alkanoylamino; substituted arylamino; substituted aralkanoylamino; thiol; alkylthio; arylthio; aralkylthio; cycloalkylthio; heterocyclothio; alkylthiono; arylthiono; aralkylthiono; alkylsulfonyl; arylsulfonyl; aralkylsulfonyl; sulfonamido (e.g., SO₂NH₂); substituted sulfonamido; nitro; cyano; carboxy; carbamyl (e.g., CONH₂); substituted carbamyl (e.g., —C(═O)NRR′ where R and R′ are each independently hydrogen, alkyl, aryl, aralkyl and the like); alkoxycarbonyl, aryl, substituted aryl, guanidino, and heterocyclo such as indoyl, imidazolyl, furyl, thienyl, thiazolyl, pyrrolidyl, pyridyl, pyrimidyl and the like. Where applicable, the substituent may be further substituted such as with, alkyl, alkoxy, aryl, aralkyl, halogen, hydroxy and the like.

The terms “alkylaryl” or “arylalkyl” refer to an aryl group with an aliphatic substituent that is bonded to the compound through the aliphatic group. An illustrative example of an alkylaryl or arylalkyl group is benzyl, a phenyl with a methyl group that is bonded to the compound through the methyl group (—CH₂Ph where Ph is phenyl).

The term “acyl” refers to —C(═O)R where R is an aliphatic group, preferably a C₁-C₆ moiety.

The term “alkoxy” refers to —OR wherein O is oxygen and R is an aliphatic group.

The term “alkylester” refers to —OC(═O)R where R is an aliphatic group.

The term “aminoalkyl” refers to —RNH₂ where R is an aliphatic moiety.

The term “alkylamino” refers to —NHR where R is an aliphatic moiety.

The terms “halogen,” “halo”, or “halide” refer to fluorine, chlorine, bromine and iodine.

The term “haloalkyl” refers to —RX where R is an aliphatic moiety and X is one or more halogens.

The term “heterocycle or heterocyclo” refers to a cyclic aliphatic (preferably a five- or six-membered ring) whose cyclic structure includes one or more heteroatoms such as N, O, and S

The term “hydroxyalkyl” refers to —ROH where R is an aliphatic moiety.

The term “isolated” as used herein to refer to a compound of the present invention, means altered “by human intervention from its natural state. For example, if the compound occurs in nature, it has been changed or removed from its original environment, or both. In other words, a compound naturally present in a living organism is not “isolated,” but the same compound separated from the coexisting materials of its natural state is “isolated”. However, with respect to compounds found in nature, the compound is isolated if that compound is substantially free of the materials with which that compound is associated in its natural state.

The term “oxo” refers to a carbonyl oxygen (═O).

The term “sixteen-membered macrolide” is a cyclic lactone whose backbone comprises 15 carbon atoms and an oxygen atom. The term sixteen-membered macrolactone is also a cyclic lactone whose backbone comprises 15 carbon atoms and an oxygen atom. The term macrolide encompasses the term macrolactone as macrolactone is generally used herein to specify the aglycone form of a macrolide.

The term “purified” as it refers to a compound means that the compound is in a preparation that is substantially free of contaminating or undesired materials. The term purified can also mean that the compound forms a major component of the preparation, such as constituting about 50%, about 60%, about 70%, about 80%, about 90%, about 95% or more by weight of the components in the preparation.

The term “subject” as used herein, refers to an animal, preferably a mammal that has been the object of treatment, observation or experiment or a human who has been the object of treatment and/or observation.

The term “therapeutically effective amount” as used herein, means that amount of active compound or pharmaceutical agent that elicits the biological or medicinal response in a tissue system, animal or human that alleviates the symptoms of or otherwise ameliorates or treats the disease or disorder being treated.

The term “composition” is intended to encompass a product comprising the specified ingredients in the specified amounts, as well as any product that results, directly or indirectly, from combinations of the specified ingredients in the specified amounts.

The term “pharmaceutically acceptable salt” is a salt suitable for pharmaceutical formulation and/or administration to a subject. Suitable pharmaceutically acceptable salts of compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where a compound includes an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate). Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like.

The term “pharmaceutically acceptable carrier” is a medium that is used to prepare a desired dosage form of a compound. A pharmaceutically acceptable carrier includes solvents, diluents, or other liquid vehicle; dispersion or suspension aids; surface active agents; isotonic agents; thickening or emulsifying agents, preservatives; solid binders; lubricants and the like. Remington's Pharmaceutical Sciences, Fifteenth Edition, E. W. Martin (Mack Publishing Co., Easton, Pa., 1975) and Handbook of Pharmaceutical Excipients, Third Edition, A. H. Kibbe, ed. (Amer. Pharmaceutical Assoc. 2000), both of which are incorporated herein by reference in their entireties, disclose various carriers used in formulating pharmaceutical compositions and known techniques for the preparation thereof.

The term “pharmaceutically acceptable ester” is an ester that hydrolzyes in vivo into the intended compound or a salt thereof. Illustrative examples of suitable ester groups include, for example, those derived from pharmaceutically acceptable aliphatic carboxylic acids such as formates, acetates, propionates, butyrates, acrylates, and ethylsuccinates.

Compounds of the Present Invention

In one aspect of the present invention, sixteen-membered macrolide possessing a side chain Z are provided where Z is aliphatic, aryl, alkylaryl, halide, ═NOR³, ═NNHR³, or —W—R³ where W is O, S, NC(═O)R⁴, NC(═O)OR⁴, NC(═O)NHR⁴ or NR⁴, where R³ and R⁴ are each independently hydrogen, aliphatic, aryl or alkylaryl. In one embodiment R³ and R⁴ each are independently selected from a group consisting of: hydrogen, C₁-C₁₀ alkyl; C₂-C₁₀ alkenyl; C₂-C₁₀ alkynyl; C₁-C₁₀ haloalkyl; C₁-C₁₀ hydroxyalkyl; C₁-C₁₀ azidoalkyl; C₁-C₁₀ aminoalkyl; C₁-C₁₀ alkylamino; —(CH₂)ₙ-cycloalkyl; —(CH₂)ₙ-heterocyclo; —(CH₂)-aryl; —(CH₂)ₙ—CH—CH-aryl; —(CH₂)ₙ—CH═CH—CH₂-aryl; —(CH₂)ₙ—NHC(═O)—(CH₂)ₘ-aryl where n and m are each independently 0-5. In another embodiment, Z is selected from the group consisting of: —O—(CH₂)ₙ-cycloalkyl; —O—(CH₂)ₙ-heterocyclo; —O—(CH₂)ₙ-aryl; —O—(CH₂)ₙ—CH═CH-aryl; and —O—(CH₂)ₙ—CH═CH—CH₂-aryl where n is 0-5. In another embodiment, the term “aryl” in any of the above descriptions of Z, is phenyl or naphthyl. In yet another embodiment, the term “aryl” in any of the above descriptions of Z, is selected from the group consisting of:

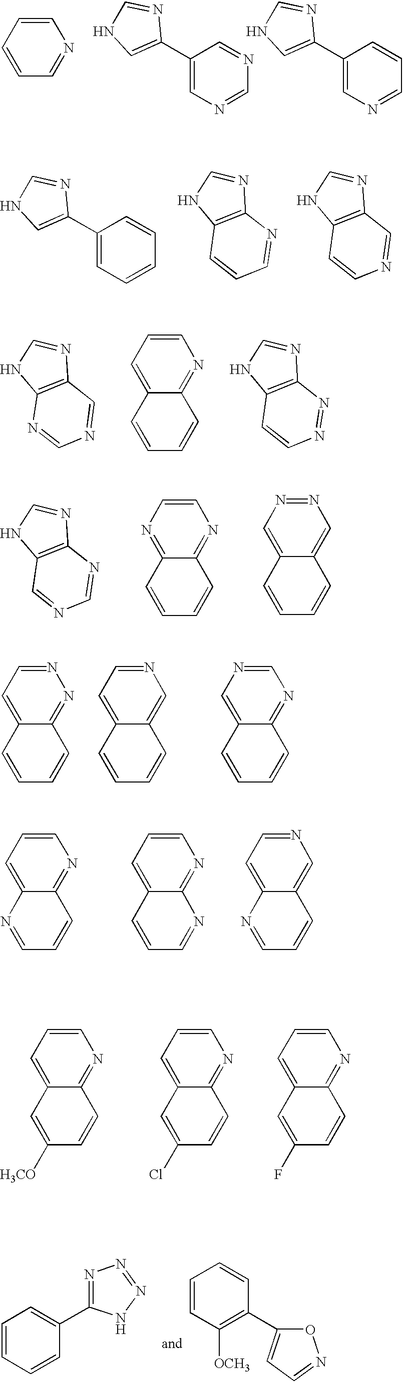

where the aryl moiety is attached at any suitable position.

In another embodiment, Z is attached to C-15 and R³ and R⁴ each are independently selected from a group consisting of: hydrogen, C₃-C₁₀ alkyl; C₂-C₁₀ alkenyl; C₂-C₁₀ alkynyl; C₁-C₁₀ haloalkyl; C₁-C₁₀ hydroxyalkyl; C₁-C₁₀ azidoalkyl; C₁-C₁₀ aminoalkyl; C₁-C₁₀ alkylamino; —(CH₂)ₙ-cycloalkyl; —(CH₂)ₙ-heterrocyclo; —(CH₂)ₙ-aryl; —(CH₂)ₙ—CH═CH-aryl; —(CH₂)ₙ—CH═CH—CH₂-aryl; —(CH₂)ₙ—NHC(═O)—(CH₂)ₘ-aryl where n and m are each independently 0-5.

In one embodiment Z is attached to C-7, C-11, or C-13 of the macrolide. In another embodiment Z is attached to C-8 of the macrolide. In another embodiment, Z is attached to C-6 of the macrolide. In another embodiment Z is attached to a substituent at C-6 or at C-14. In another embodiment, Z is attached to C-12 of the macrolide. In yet another embodiment, Z is attached to C-3 or C-9 of the macrolide.

In another aspect of the present invention, bicyclic compounds are provided wherein one of the cyclic components is a sixteen-membered macrolide and the other is a cyclic moiety whose cyclic structure is formed by between 3 and 10 atoms. In one embodiment, the cyclic moiety is attached to the macrolide in the syn-configuration. In another embodiment, the cyclic moiety is a five membered ring. In another embodiment, the cyclic moiety is a six membered ring. In yet another embodiment, the cyclic moiety is a heterocycle. In another embodiment, the cyclic moiety is attached to the macrolide at non-adjacent carbons of the macrolide. In another embodiment, the cyclic moiety is attached to the macrolide at adjacent carbons of the macrolide.

In yet another embodiment, the cyclic moiety is selected from the group consisting of

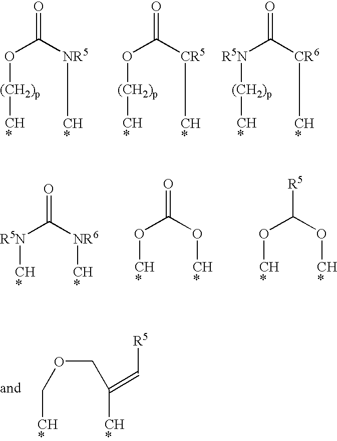

wherein:

* is the attachment site of the cyclic moiety to the macrolide;

p is an integer from 0 to 3; and,

R⁵ and R⁶ are each independently selected from the group consisting of hydrogen, aliphatic, aryl and alkylaryl.

In another embodiment, the cyclic moiety is attached to the macrolide in a syn-configuration. In another embodiment, p is 0 or 1; and R⁵ and R⁶ are each independently selected from the group consisting of hydrogen, aliphatic, aryl and alkylaryl. In yet another embodiment, p is 0 or 1; and R⁵ and R⁶ are each independently selected from the group consisting of: hydrogen C₁-C₁₀ alkyl; C₂-C₁₀ alkenyl; C₂-C₁₀ alkynyl; C₁-C₁₀ haloalkyl; C₁-C₁₀ hydroxyalkyl; C₁-C₁₀ aminoalkyl; C₁-C₁₀ alkylamino; —(CH₂)ₙ-cycloalkyl; —(CH₂)ₙ-heterrocyclo; —(CH₂)ₙ-aryl; —(CH₂)ₙ—CH═CH-aryl; —(CH₂)ₙ—CH═CH—CH₂-aryl; —(CH₂)ₙ—NHC(═O)—(CH₂)ₘ-aryl where n and m are each independently 0-5.

In yet another embodiment, the term “aryl” in any of the descriptions of R⁵ and R⁶ is phenyl or naphthyl. In another embodiment, the term “aryl” in any of the descriptions of R⁵ and R⁶ is selected from the group consisting of

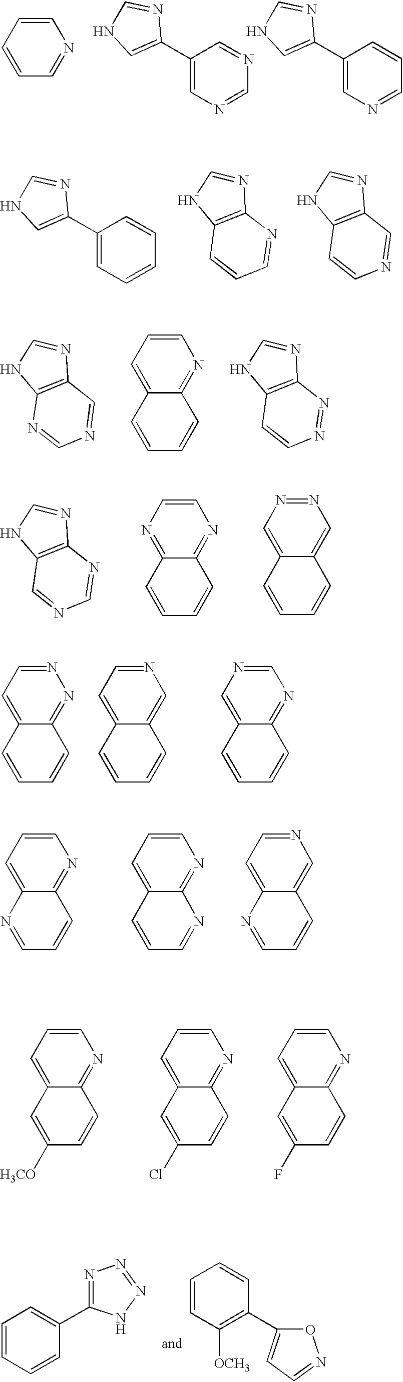

where the aryl moiety may be attached at any suitable position.

In another embodiment, the cyclic moiety is attached to the macrolide at C-9 and at C-11. In another embodiment, the cyclic moiety is attached to the macrolide at C-11 and at C-13. In another embodiment, the cyclic moiety is attached to the macrolide at C-13 and C-14. In another embodiment, the cyclic moiety is attached to the macrolide at C-11 and C-12. In another embodiment, the cyclic moiety is attached to the macrolide at C-10 and C-11. In another embodiment, the cyclic moiety is attached to the macrolide at C-9 and C-10. In another embodiment, the cyclic moiety is attached to the macrolide at C-6 and C-7.

In another aspect of the present invention, sixteen-membered macrolides are provided that possesses a side chain Z and that bind to the domain II region of the 23 S RNA wherein Z is as previously defined. In one embodiment, the macrolide also inhibits the peptidyl transferase activity of the 23 RNA. In another embodiment, the macrolide also possesses a side chain Z′ where Z′ is an aryl- or a saccharide-containing aliphatic. In yet another embodiment, Z′ is a disaccharide, preferably a 4-mycarosyl-mycaminose wherein one or more hydroxyls of the mycarose has been converted into an aliphatic or aryl containing moiety.

In another aspect of the present invention, bicyclic compounds are provided wherein one of the cyclic components is a sixteen-membered macrolide and the other is a cyclic moiety whose cyclic structure is formed by between 3 and 10 atoms and that bind to the domain II region of the 23 S RNA, wherein the cyclic moiety is as previously defined. In one embodiment, the compound also inhibits the peptidyl transferase activity of the 23 RNA. In another embodiment, the compound also possesses a side chain Z′ where Z′ is an aryl- or a saccharide-containing aliphatic. In yet another embodiment, Z′ is a disaccharide, preferably a 4-mycarosyl-mycaminose wherein one or more hydroxyls of the mycarose has been converted into an aliphatic or aryl containing moiety.

In another aspect of the present invention, compound are provided of the formula

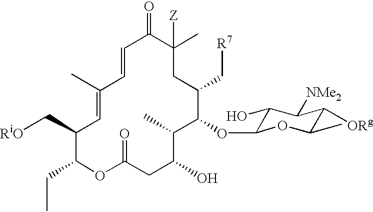

where Rⁱ is hydrogen, or mycinose;

R^{g} is hydrogen, mycarose, 4-acyl-mycarose, or 4-sulfonyl-mycarose;

R⁷ is hydrogen, methyl, hydroxymethyl, aminomethyl or CHO; and,

Z is selected from the group consisting of: hydrogen, —O—(CH₂)ₙ-cycloalkyl; —O—(CH₂)ₙ-heterrocyclo; —O—(CH₂)ₙ-aryl; —O—(CH₂)ₙ—CH═CH-aryl; and —O—(CH₂)ₙ—CH═CH—CH₂-aryl where n is 0-5.

In another aspect of the present invention, compounds are provided of the formula

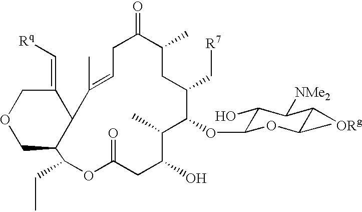

wherein

R⁷is hydrogen, methyl, hydroxymethyl, aminomethyl or CHO; and,

R^{g} is hydrogen, mycarose or 4-acyl-mycarose; and,

R^{q} is C₁-C₅ alkyl, aryl, —(CH₂)ₙ-cycloalkyl; —(CH₂)ₙ-heterrocyclo; —(CH₂)ₙ-aryl; —(CH₂)ₙ—CH═CH-aryl; —(CH₂)ₙ—CH═CH—CH₂-aryl; —(CH₂)ₙ—NHC(═O)—(CH₂)ₘ-aryl where n and m are each independently 0-5.

In another aspect of the present invention, compounds are provided of the formula

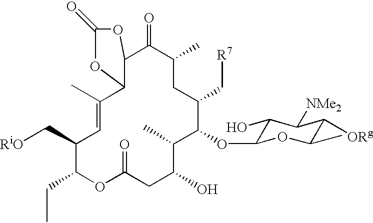

wherein

Rⁱ is hydrogen or mycinose;

R^{g} is hydrogen, mycarose or 4-acyl-mycarose; and,

R⁷ is hydrogen, methyl, hydroxymethyl, aminomethyl or CHO.

In another aspect of the present invention, compounds are provided of the formula:

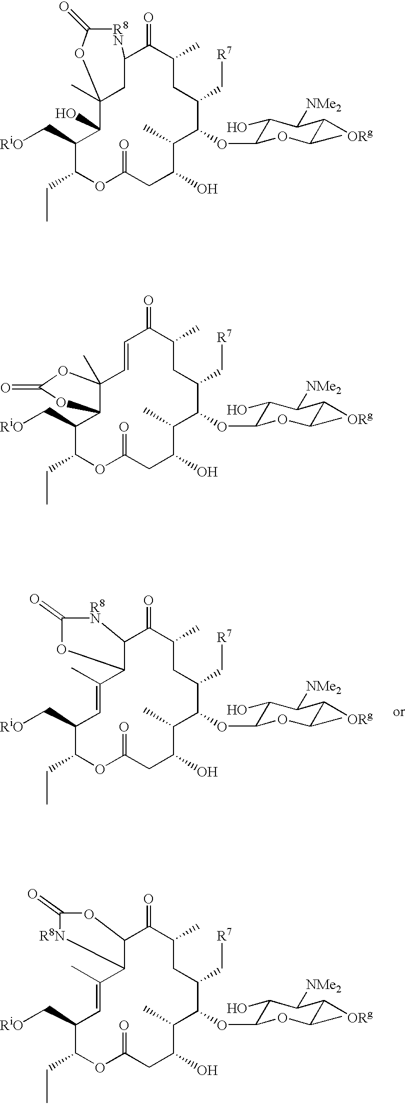

wherein

Rⁱ is hydrogen or mycinose;

R^{g} is hydrogen, mycarose or 4-acyl-mycarose;

R⁷ is hydrogen, methyl, hydroxymethyl, aminomethyl or CHO; and,

R⁸ is hydrogen, C₁-C₅ alkyl, aryl, —(CH₂)ₙ-cycloalkyl; —(CH₂)ₙ-heterrocyclo; —(CH₂)ₙ-aryl; —(CH₂)ₙ—CH═CH-aryl; —(CH₂)ₙ—CH═CH—CH₂-aryl; and —(CH₂)ₙ—NHC(═O)—(CH₂)ₘ-aryl where n and m are each independently 0-5.

In another aspect of the present invention, compounds are provided of the formula

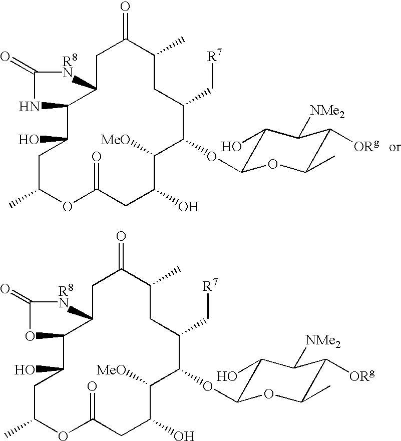

wherein

R^{g} is hydrogen, mycarose or 4-acyl-mycarose;

R⁷ is hydrogen, methyl, hydroxymethyl, aminomethyl or CHO; and,

R⁸ is hydrogen, C₁-C₅ alkyl, aryl, —(CH₂)ₙ-cycloalkyl; —(CH₂)ₙ-heterrocyclo; —(CH₂)ₙ-aryl; —(CH₂)ₙ—CH═CH-aryl; —(CH₂)ₙ—CH═CH—CH₂-aryl; and —(CH₂)ₙ—NHC(═O)—(CH₂)ₘ-aryl where n and m are each independently 0-5.

Starting Materials

Starting materials for the attachment of side chains Z or Z′, or for the attachment of the cyclic moiety can be any sixteen-membered macrolide. In certain embodiments, the starting material is a macrolide produced by a naturally occurring host organism. In another embodiment, the starting material is a macrolide produced by a recombinant organism. In another embodiment, the starting material is a macrolide isolated from a naturally occurring or recombinant host organism that has been subject to further modification using chemical or biochemical means or both.

The following discussion serves to illustrate the diversity of starting materials that may be used in the practice of the present invention. Where applicable, the discussions of polyketide biosynthesis and the modifications that can be made to the host organisms or to the sixteen-membered macrolide compound are described with reference to tylosin for the purposes of illustration. However, as can be appreciated by those skilled in the art upon consideration of the present invention, any naturally occurring sixteen-membered macrolide can be substituted for tylosin in accordance with the invention.

PKS Biosynthesis

Tylosin (1), is a naturally occurring sixteen-membered macrolide whose structure is shown below,

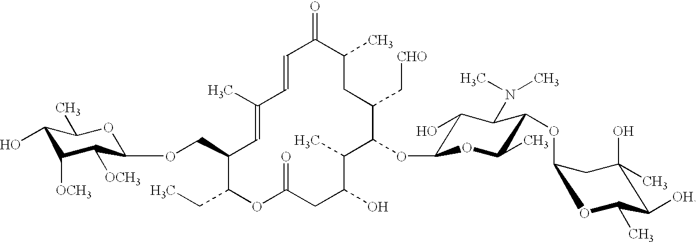

The polyketide portion of tylosin is the product of a modular polyketide synthase enzyme (“PKS”), a large protein comprising multiple subunits and enzymatic active sites that is encoded by multiple genes (or a single gene with multiple open reading frames); these genes are located together on the chromosome and are collectively referred to as the PKS gene cluster. A number of products (e.g., saccharides) and enzymes (e.g., epoxidase and glycotransferase) modify the macrolactone product of the PKS. The genes for the PKS, accessory products and enzymes are generally contiguous and are collectively referred to as the macrolide biosynthetic gene cluster. A schematic illustration of the tylosin biosynthetic gene cluster is below

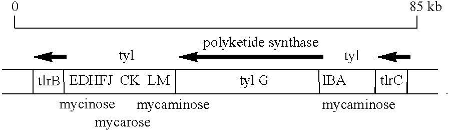

Modular PKS enzymes such as the tylosin PKS, generally contain (i) a loading module, (ii) a number of extender modules, (iii) and a releasing domain (which is also called a thioesterase domain) and are organized into distinct units (or modules) that control the structure of a discrete two-carbon portion of the polyketide. The two-carbon units from which polyketides are synthesized are of the general formula (R—C(═O)) and can be referred to as starters or extenders depending on whether the two carbon unit initiates the synthesis of the polyketide or extends (adds to) the growing polyketide chain during synthesis. The term “polyketide” refers to a polymer of two-carbon unit monomers or ketides (R—C(═O)). As their names suggest, starters bind to the loading module and initiate polyketide synthesis, and extenders bind to the extender modules and extend the starter as is the case with the first extender module or extend the growing polyketide chain as is the case with the other extender modules. Starters and extenders are typically acylthioesters, most commonly acetyl CoA, propionyl CoA and the like for starter units and malonyl CoA, methylmalonyl CoA, methoxymalonyl CoA, hydroxymalonyl CoA, and ethylmalonyl CoA and the like for extender units. Other building blocks, can be used, however, such as for example, 2-allylmalonyl CoA and amino acid-like acylthioesters.

Each extender module of a modular PKS contains three core domains needed for polyketide synthesis: an acyltransferase (“AT”) responsible for selecting and binding the appropriate extender unit; an acyl-carrier protein (“ACP”) responsible for carrying the growing polyketide chain; and a β-ketoacyl ACP synthase (“KS”) responsible for condensing the extender unit to the growing polyketide chain. Together, these core domains add a two-carbon β-ketothioester onto the growing end of the polyketide chain. An extender module that only contains these three core domains is termed a “minimal module.” A loading module may contain only an AT and ACP domain or may contain these domains and a KS domain as with the minimal extender module.

In addition, a module may contain additional domains. These include a set of reductive cycle domains responsible for modifying the β-ketone produced by the core domains of the previous extender module (or the loading module in the case of the first extender module). If present, a ketoreductase (“KR”) domain reduces the previously added keto group to an alcohol (—C(═O)—CH₂— into —C(OH)—CH₂). If present with a KR, a dehydratase (“DH”) dehydrates the alcohol into a double bond (—C(OH)—CH₂— to —CH═CH—). If present with a DH and a KR, an enoylreductase (“ER”) reduces the double bond to an alkane (—CH═CH— to —CH₂CH₂—). Other types of variable domains include methyltransferase (MT) and O-methyltransferase (“OMT”) that also further modify the previously added two-carbon unit. MT domains add a methyl group, typically from S-adenosylmethionine to the α-carbon of the previously-added two-carbon unit, and OMT domains add the methyl group to the oxygen atom of the enol form of the β-ketothioester to form a methyl vinyl ether, or to the alcohol resulting from the action of a KR domain so as to form a methyl ether.

The PKS gene cluster generally includes more than one gene or open reading frame and these genes do not always follow the order in which they function. The order of modules as they are encoded within a gene appears to follow the order in which they function in the biosynthesis. The order of domains within a module, while conserved between PKSs, does not appear to follow the order in which they function in the biosynthesis. The boundaries of domains can be deduced from the high homology that generally exists between the many known examples of PKS domains. The regions between the domains are called linker regions.

As can be appreciated by those skilled in the art, polyketide biosynthesis can be manipulated to make a product other than the product of a naturally occurring PKS biosynthetic cluster. For example, AT domains can be altered or replaced to change specificity. The variable domains within a module can be deleted and or inactivated or replaced with other variable domains found in other modules of the same PKS or from another PKS. See e.g., Katz & McDaniel, Med Res Rev 19: 543-558 (1999) and WO 98/49315 which are each incorporated herein by reference. Similarly, entire modules can be deleted and/or replaced with other modules from the same PKS or another PKS. See e.g., Gokhale et al., Science 284: 482 (1999) and WO 00/47724 which are each incorporated herein by reference. Protein subunits of different PKSs also can be mixed and matched to make compounds having the desired backbone and modifications. For example, subunits of 1 and 2 (encoding modules 1-4) of the pikromycin PKS were combined with the DEBS3 subunit to make a hybrid PKS product. See Tang et al., Science, 287: 640 (2001), WO 00/26349 and WO 99/61599 which are each incorporated herein by reference.

Table 1 lists illustrative examples of macrolide biosynthetic gene clusters that have been sequenced in whole or in part, and the publications in which they are described. All of these publications are incorporated herein by reference. The domains, modules and subunits that are described by the PKS genes listed in Table 1 as well as the genes for polyketide modification or tailoring enzymes are among those that can be used in the practice of the present invention.

TABLE 1

| PKS or | |
|---|---|
| PKS Tailoring | |
| Enzyme Genes | Publications |
| Avermectin | U.S. Pat. No. 5,252,474; U.S. Pat. No. 4,703,009; and EP Pub. No. |
| | 118,367 to Merck. |
| | MacNeil et al., 1993, Industrial Microorganisms: Basic and |
| | Applied Molecular Genetics, Baltz, Hegeman, & Skatrud, eds. (ASM), pp. |
| | 245-256, A Comparison of the Genes Encoding the Polyketide Synthases |
| | for Avermectin, Erythromycin, and Nemadectin. |
| | MacNeil et al., 1992, Gene 115: 119-125, Complex Organization |
| | of the Streptomyces avermitilis genes encoding the avermectin polyketide |
| | synthase. |
| | Ikeda and Omura, 1997, Chem. Res. 97: 2599-2609, Avermectin |
| | biosynthesis. |
| Candicidin (FR008) | Hu et al., 1994, Mol. Microbiol. 14: 163-172. |
| Epothilone | PCT Pub. No. 99/66028 to Novartis. |
| | PCT Pub. No. 00/31247 to Kosan. |
| Erythromycin | PCT Pub. No. 93/13663; U.S. Pat. No. 6,004,787; and U.S. Pat. |
| | No. 5,824,513 to Abbott. |
| | Donadio et al., 1991, Science 252:675-9. |
| | Cortes et al., 8 Nov. 1990, Nature 348: 176-8, An unusually large |
| | multifunctional polypeptide in the erythromycin producing polyketide |
| | synthase of Saccharopolyspora erythraea. |
| | Glycosylation Enzymes |
| | PCT Pub. No. 97/23630 and U.S. Pat. No. 5,998,194 to Abbott. |
| FK-506 | Motamedi et al., 1998, The biosynthetic gene cluster for the |
| | macrolactone ring of the immunosuppressant FK-506, Eur. J. biochem. |
| | 256: 528-534. |
| | Motamedi et al., 1997, Structural organization of a multifunctional |
| | polyketide synthase involved in the biosynthesis of the macrolide |
| | immunosuppressant FK-506, Eur. J. Biochem. 244: 74-80. |
| | Methyltransferase |
| | U.S. Pat. No. 5,264,355 and U.S. Pat. No. 5,622,866 to Merck. |
| | Motamedi et al., 1996, Characterization of methyltransferase and |
| | hydroxylase genes involved in the biosynthesis of the immunosuppressants |
| | FK-506 and FK-520, J. Bacteriol. 178: 5243-5248. |
| FK-520 | PCT Pub. No. 00/20601 to Kosan. |
| | Nielsen et al., 1991, Biochem. 30: 5789-96. |
| Lovastatin | U.S. Pat. No. 5,744,350 to Merck. |
| Nemadectin | MacNeil et al., 1993, supra. |
| Niddamycin | PCT Pub. No. 98/51695 to Abbott. |
| | Kakavas et al., 1997, Identification and characterization of the |
| | niddamycin polyketide synthase genes from Streptomyces caelestis, J. |
| | Bacteriol. 179: 7515-7522. |
| Oleandomycin | Swan et al., 1994, Characterisation of a Streptomyces antibioticus |
| | gene encoding a type I polyketide synthase which has an unusual coding |
| | sequence, Mol. Gen. Genet. 242: 358-362. |
| | U.S. Patent No. 6,251,636; PCT Pub. No. 00/026349 to Kosan. |
| | Olano et al., 1998, Analysis of a Streptomyces antibioticus |
| | chromosomal region involved in oleandomycin biosynthesis, which |
| | encodes two glycosyltransferases responsible for glycosylation of the |
| | macrolactone ring, Mol. Gen. Genet. 259(3): 299-308. |
| | PCT Pub. No. 99/05283 to Hoechst. |
| Picromycin | PCT Pub. No. 99/61599 to Kosan. |
| | PCT Pub. No. 00/00620 to the University of Minnesota. |
| | Xue et al., 1998, Hydroxylation of macrolactones YC-17 and |
| | narbomycin is mediated by the pikC-encoded cytochrome P450 in |
| | Streptomyces venezuelae, Chemistry & Biology 5(11): 661-667. |
| | Xue et al., Oct. 1998, A gene cluster for macrolide antibiotic |
| | biosynthesis in Streptomyces venezuelae: Architecture of metabolic |
| | diversity, Proc. Natl. Acad. Sci. USA 95: 12111 12116. |
| Platenolide | EP Pub. No. 791,656; and U.S. Pat. No. 5,945,320 to Lilly. |
| Rapamycin | Schwecke et al., Aug. 1995, The biosynthetic gene cluster for the |
| | polyketide rapamycin, Proc. Natl. Acad. Sci. USA 92: 7839-7843. |
| | Aparicio et al., 1996, Organization of the biosynthetic gene cluster |
| | for rapamycin in Streptomyces hygroscopicus: analysis of the enzymatic |
| | domains in the modular polyketide synthase, Gene 169: 9-16. |
| Rifamycin | PCT Pub. No. 98/07868 to Novartis. |
| | August et al., 13 Feb. 1998, Biosynthesis of the ansamycin |
| | antibiotic rifamycin: deductions from the molecular analysis of the rif |
| | biosynthetic gene cluster of Amycolatopsis mediterranei S669, Chemistry |
| | & Biology, 5(2): 69-79. |
| Sorangium PKS | U.S. Pat. Nos. 6,280,999 and 6,090,601 to Kosan. |
| Soraphen | U.S. Pat. No. 5,716,849 to Novartis. |
| | Schupp et al., 1995, J. Bacteriology 177: 3673-3679. A Sorangium |
| | cellulosum (Myxobacterium) Gene Cluster for the Biosynthesis of the |
| | Macrolide Antibiotic Soraphen A: Cloning, Characterization, and |
| | Homology to Polyketide Synthase Genes from Actinomycetes. |
| Spinocyn | PCT Pub. No. 99/46387 to DowElanco. |
| Spiramycin | U.S. Pat. No. 5,098,837 to Lilly. |
| | Activator Gene |
| | U.S. Pat. No. 5,514,544 to Lilly. |
| Tylosin | U.S. Pat. No. 5,876,991; U.S. Pat. No. 5,672,497; U.S. Pat. No. |
| | 5,149,638; EP Pub. No. 791,655; and EP Pub. No. 238,323 to Lilly. |
| | Kuhstoss et al., 1996, Gene 183: 231-6., Production of a novel |
| | polyketide through the construction of a hybrid polyketide synthase. |
| | Tailoring enzymes |
| | Merson-Davies and Cundliffe, 1994, Mol. Microbiol. 13: 349-355. |
| | Analysis of five tylosin biosynthetic genes from the ty1BA region of the |
| | Streptomyces fradiae genome. |

As can be appreciated by those skilled in the art, a wide variety of domains, modules, protein subunits as well as whole proteins are available from known PKS biosynthetic clusters that can be used to make alterations in the biosynthesis of a sixteen-membered macrolide.

Tylosin Biosynthesis

The tylosin PKS (the product of the tylG PKS gene cluster) makes a macrolactone called tylactone (2). A graphical representation of the tylosin PKS and the biosynthesis of tylactone is shown below.

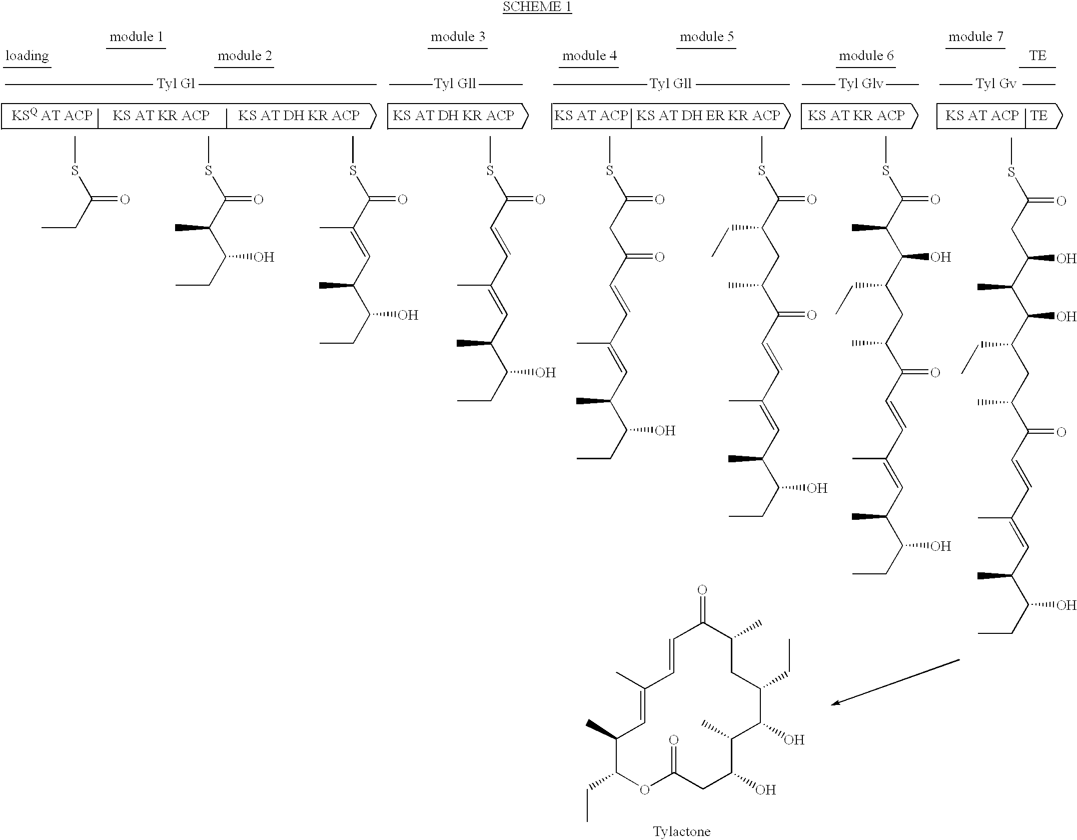

The ATs in modules 1, 2, 4, and 6 resulting in methyl side chains at C-14, C-12, C-8, and C-4 respectively, each specify methylmalonyl-CoA. The ATs in module 3 and 7 resulting in hydrogens at C-10 and C-2 respectively, each specify a malonyl-CoA. The AT in module 5 specifies an ethylmalonyl CoA resulting in an ethyl at C-6.

The reductive cycle domains (KR; KR & DH; or KR, DH & ER) when present modify the keto group of the previously added two carbon unit. For example, module 1 includes a KR and reduces the keto group that was added by the loading and processing of the starter unit (which is methylmalonyl CoA). The resulting hydroxyl group is involved in the cyclization reaction and results in the lactone oxygen. Modules 2 and 3 each have both a KR and a DH and so result in a double bond within the previously added two carbon unit (from modules 1 and 2 respectively). Module 4 has an inactive KR, so the keto group at C-9 remains unmodified. Module 5 has a KR, DH, and an ER so results in a fully reduced state (—CH₂—) at C-7. Modules 6 and 7 each have a KR and so results in the reduction of a keto group to a hydroxyl at each of the C-5 and C-3 positions.

As illustrated by Scheme 2, tylactone (2) is subsequently modified in a number of post-PKS biochemical transformations by polyketide modification enzymes to yield tylosin (1). Genes encoding the various enzymes are indicated.

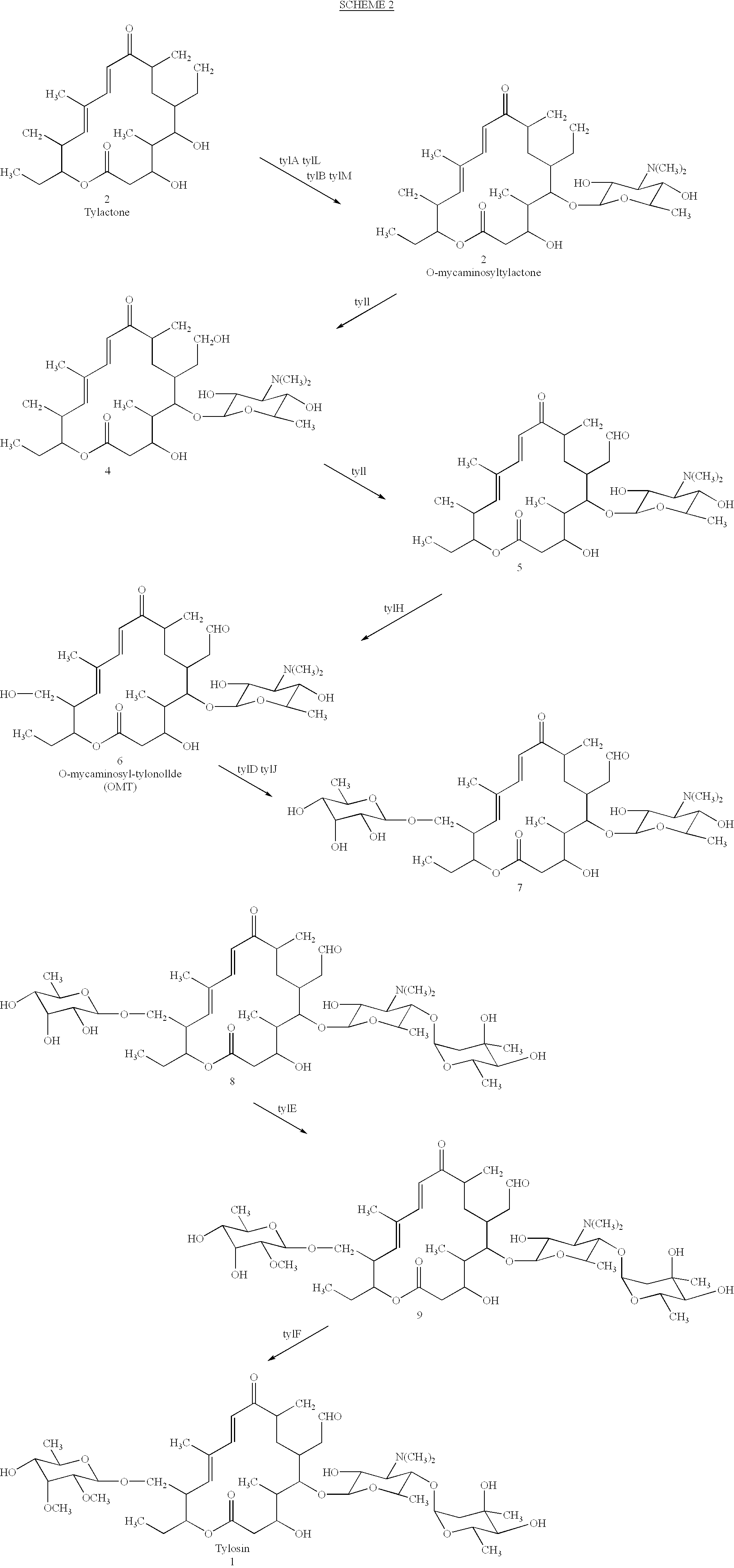

As shown in Scheme 2, the amino sugar mycaminose is added to the C-5 hydroxyl of tylactone (2) to make O-mycaminosyltylactone (3). The core cyclic lactone of O-mycaminosyltylactone is oxidized at two positions. The first oxidation is the conversion of the C-20 methyl to methylalcohol (4) and then to CHO (5) and the second oxidation is the conversion of the C-23 methyl to methylalcohol (6). Next, 6-deoxyallose is added to the C-23 hydroxyl, and mycarose is added to the C-4″ hydroxyl. Finally, tylosin (1) results when the C-2′″ and C-3′″ hydroxyls of 6-deoxyallose are dimethylated to convert the 6-deoxyallose to mycinose.

Other naturally occurring sixteen-membered macrolides are made in a similar manner as tylosin differing only in the specific cyclic lactone that is made by the PKS enzyme and the nature of the subsequent modifications. Notably, despite the over 200 different sixteen-membered macrolides that have been characterized to date, only about six different macrolactones are needed to generate this diversity. These six different macrolactones are referred herein as Types I-VI and are described in greater detail below.

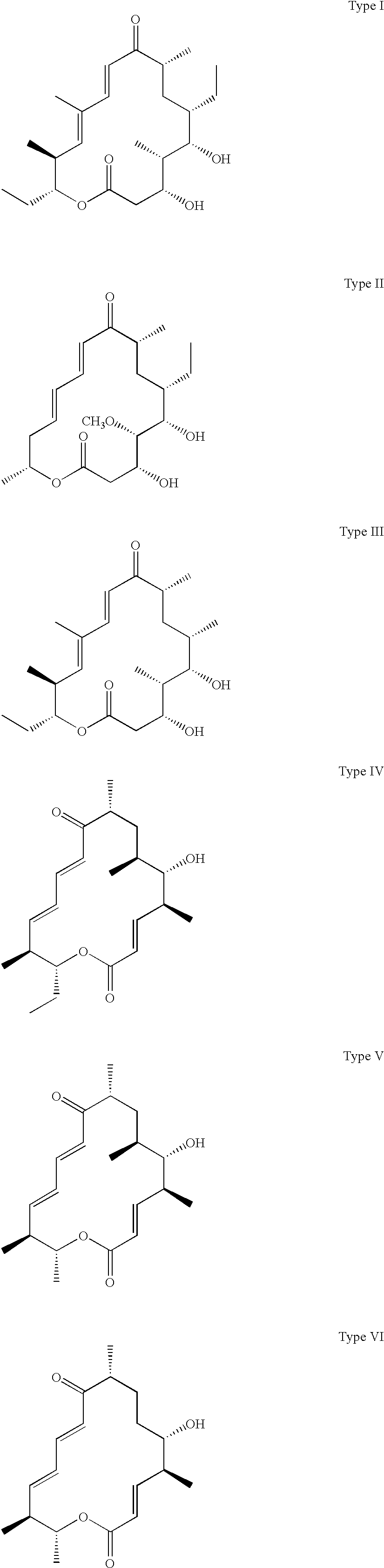

Although these six macrolactones can be used to classify all of the known sixteen-membered macrolides, two subtypes that differ from the Type V lactone skeleton in the reduction of one of its double bonds are noteworthy.

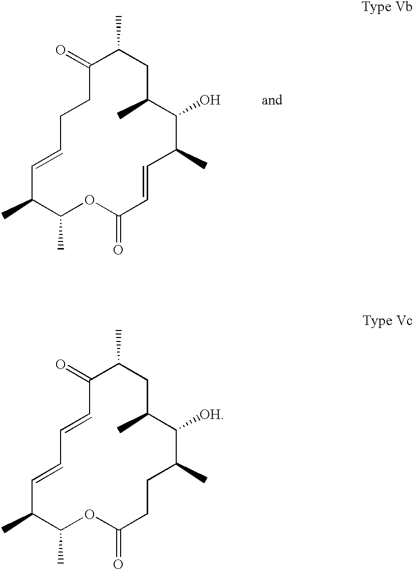

These cyclic lactones are classified as subtypes of the type V macrolactone because each only differs from the Type V structure in that one of the three double bonds present in the Type V structure is not present in these subtype macrolactones. The Type Vb and Type Vc macrolactones may be products of a PKS enzyme (having KR, DH and ER domains instead of only KR and DH domains in the appropriate module). However because sixteen-membered macrolide antibiotics based on these macrolactones are usually found as minor products in host organisms that also make Type V sixteen-membered macrolide antibiotics, the reduction of the double bond may be a post-PKS event.

Table 2 contains an illustrative list of sixteen-membered macrolides, the natural producer organism that makes it in nature, and the type of macrolactone (I, II, III, IV, V, and VI) it possess.

TABLE 2

| | | Molecular | |
|---|---|---|---|
| Macrolide | Main Producer | Formula | Type |
| A-6888 C | S. flocculus | C₃₇H₆₁NO₁₂ | I |
| A-6888 X | S. flocculus | C₃₇H₆₁NO₁₂ | I |
| Acumycin | S. griseofiavus | C₃₇H₅₉NO₁₂ | I |
| Aldgamycin C | S. lavendulae | C₃₅H₅₉O₁₅ | Vc |
| Aldgamycin E | S. lavendulae | C₃₇H₅₈NO₁₅ | V |
| Aldgamycin F | S. lavendulae | C₃₇H₆₃NO₁₂ | V |
| Angolamycin | S. eurythermus | C₄₆H₇₇NO₁₇ | I |
| B-5050 G | S. hygroscopicus | C₄₂H₆₉NO₁₆ | II |
| Carbomycin A | S. halstedii | C₄₂H₆₇NO₁₆ | II |
| Carbomycin B | S. halstedii | C₄₂H₆₇NO₁₅ | II |
| Chalcomycin | S. albogriseolus | C₃₅H₅₆NO₁₄ | V |
| Cirramycin A₁ | S. cirratus | C₃₁H₅₁NO₁₀ | I |
| Deltamycin A₁ | S. deltae | C₃₉H₆₁NO₁₆ | II |
| Deltamycin A₂ | S. deltae | C₄₀H₆₃NO₁₆ | II |
| Deltamycin A₃ | S. deltae | C₄₁H₆₅NO₁₆ | II |
| DHP | S. platensis | C₃₈H₆₃NO₁₄ | II |
| 20-Dihydro- | Streptomyces sp. SK-62 | C₄₆H₇₉O₁₇ | I |
| angolamycin | | | |
| DOA | S. platensis | C₃₇H₅₉NO₁₄ | II |
| DOP | S. platensis | C₃₈H₆₁NO₁₄ | II |
| EOA | S. platensis | C₃₇H₅₉NO₁₅ | II |
| EOP | S. platensis | C₃₈H₆₁NO₁₅ | II |
| Espinomycin A₂ | S. fungicidicus var. | C₄₂H₆₉NO₁₅ | II |
| | espinomyceticus | | |
| GERI-155 | Streptomyces sp. GERI- | C₃₅H₅₈O₁₄ | Vb |
| | 155 | | |
| Juvenimicin A₂ | M. chalcea var. | C₃₀H₅₁NO₈ | III |
| | izumensis | | |
| Juvenimicin A₄ | M. chalcea var. | C₃₁H₅₈NO₉ | I |
| | izumensis | | |
| Juvenimicin B₁ | M. chalcea var. | C₃₁H₅₃NO₈ | I |
| | izumensis | | |
| Juvenimicin B₃ | M. chalcea var. | C₃₁H₅₃NO₉ | I |
| | izumensis | | |
| Leucomycin A₁ | Stv. kitasatoensis | C₄₀H₆₇NO₁₄ | II |
| Leucomycin A₃ | Stv. kitasatoensis | C₄₂H₆₉NO₁₅ | II |
| Leucomycin A₄ | Stv. kitasatoensis | C₄₁H₆₇NO₁₅ | II |
| Leucomycin A₅ | Stv. kitasatoensis | C₃₉H₆₅NO₁₄ | II |
| Leucomycin A₆ | Stv. kitasatoensis | C₄₀H₆₅NO₁₅ | II |
| Leucomycin A₇ | Stv. kitasatoensis | C₃₈H₆₃NO₁₄ | II |
| Leucomycin A₈ | Stv. kitasatoensis | C₃₉H₆₃NO₁₅ | II |
| Leucomycin A₉ | Stv. kitasatoensis | C₃₇H₆₁NO₁₄ | II |
| Leucomycin U | Stv. kitasatoensis | C₃₇H₆₁NO₁₄ | II |
| Leucomycin V | Stv. kitasatoensis | C₃₅H₅₉NO₁₃ | II |
| M-4365 A₁ | M. capillata | C₃₁H₅₃NO₈ | I |
| M-4365 G₁ | M. capillata | C₃₁H₅₃NO₇ | I |
| M-4365 G₂ | M. capillata | C₃₁H₅₁NO₈ | I |
| Macrocin | S. fradiae | C₄₆H₇₉NO₁₇ | I |
| Maridomycin I | S. hygroscopicus | C₄₃H₇₁NO₁₆ | II |
| Maridomycin II | S. hygroscopicus | C₄₂H₆₉NO₁₆ | II |
| Maridomycin III | S. hygroscopicus | C₄₁H₆₇NO₁₆ | II |
| Maridomycin IV | S. hygroscopicus | C₄₀H₆₅NO₁₆ | II |
| Maridomycin V | S. hygroscopicus | C₄₀H₆₅NO₁₆ | II |
| Maridomycin VI | S. hygroscopicus | C₃₉H₆₃NO₁₆ | II |
| Midecamycin A₁ | S. mycarofaciens | C₄₁H₆₇NO₁₅ | II |
| Midecamycin A₂ | S. mycarofaciens | C₄₂H₆₉NO₁₅ | II |
| Midecamycin A₃ | S. mycarofaciens | C₄₁H₆₅NO₁₅ | II |
| Midecamycin A₄ | S. mycarofaciens | C₄₂H₆₇NO₁₅ | II |
| Mycinamicin I | M. griseorubida | C₃₇H₆₁NO₁₃ | IV |
| Mycinamicin II | M. griseorubida | C₃₇H₆₇NO₁₂ | IV |
| Mycinamicin III | M. griseorubida | C₃₆H₅₉NO₁₁ | IV |
| Mycinamicin IV | M. griseorubida | C₃₇H₆₁NO₁₁ | IV |
| Mycinamicin V | M. griseorubida | C₃₇H₆₁NO₁₂ | IV |
| Neutramycin | S. rimosus | C₃₄H₅₄O₁₄ | VI |
| Niddamycin | S. djakartensis | C₄₀H₆₅NO₁₄ | II |
| Platenomycin A₀ | S. platensis subsp. | C₄₄H₇₃NO₁₅ | II |
| | malvinus | | |
| Platenomycin A₁ | S. platensis subsp. | C₄₃H₇₁NO₁₅ | II |
| | malvinus | | |
| Platenomycin C₂ | S. platensis subsp. | C₄₀H₆₅NO₁₅ | II |
| | malvinus | | |
| Platenomycin W₁ | S. platensis subsp. | C₄₀H₆₉NO₁₅ | II |
| | malvinus | | |
| Platenomycin W₂ | S. platensis subsp. | C₄₄H₇₁NO₁₅ | II |
| | malvinus | | |
| Relomycin | S. hygroscopicus | C₄₆H₇₉NO₁₇ | I |
| Rosamicin | M. rosaria | C₃₁H₅₁NO₉ | I |
| Spiramycin I | S. ambofaciens | C₄₃H₇₄N₂O₁₄ | II |
| Spiramycin II | S. ambofaciens | C₄₅H₇₆N₂O₁₅ | II |
| Spiramycin III | S. ambofaciens | C₄₆H₇₈N₂O₁₅ | II |
| Spiramycin IV | S. ambofaciens | C₄₃H₇₆N₂O₁₆ | II |
| Spiramycin V | S. ambofaciens | C₄₂H₇₃NO₁₆ | II |
| Spiramycin VI | S. ambofaciens | C₄₃H₇₃NO₁₆ | II |
| Staphcoccomycin | Streptomyces sp. AS- | C₃₉H₆₅NO₁₄ | I |
| | NG-16 | | |
| Tylosin | S. fradiae | C₄₆H₇₇NO₁₇ | I |

Modifications to the Macrolactone

A PKS gene cluster that encodes a PKS that produces one of the above described macrolactones can be modified using recombinant methods to make a PKS that produces any of the other types as well as novel sixteen-membered macrolactones. The PKS gene cluster can be altered using site-specific mutation, and domain/module insertions, deletions, and replacement. In particular, one can alter (i) the AT specificity of the loading or extender modules, and ii) the number of reductive cycle domains.

Changing AT specificity alters the side chains at the even numbered carbons of the macrolactone. For example, the loading module AT of tylG specifies a methyl malonyl CoA that results in an ethyl side chain at C-16. A tylactone derivative possessing a propyl side chain at C-16 therefore can be made by altering the specificity of the loading module AT of tylG to bind ethyl malonyl CoA. Similarly, a tylactone derivative possessing a hydrogen or methoxy at C-16 can be made by altering the specificity of the loading module AT of tylG to bind malonyl CoA or methoxy malonyl CoA respectively. Similarly, the AT specificity of modules 1-7 of tylG can be altered to make tylactone derivatives having different side chains at any one or more positions: C-2, C-4, C-6, C-8, C-10, C-12 and C-14. A generalized macrolactone showing the carbon positions and the corresponding module in which the AT specifies the side chain at the indicated position is shown below.

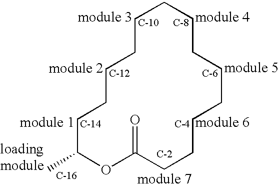

As can be seen, the AT specificity of the loading module determines the side chain at C-16 and the AT specificity of extender modules 1, 2, 3, 4, 5, 6 and 7 determines the side chain at C-14, C-12, C-10, C-6, C-8, C-4 and C-2 respectively.

One can also alter the degree of keto group processing at the odd numbered carbons. A generalized macrolactone showing the odd numbered carbon positions and the module whose number of reductive cycle domains (none; KR; KR & DH; or KR, DH & ER) is responsible the degree of keto group modification is shown below.

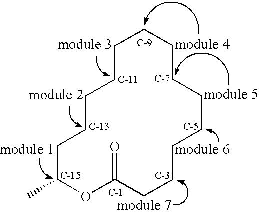

If a module is a minimal module, then the β-carbon remains a keto group. If a module is a minimal module plus a KR, then the affected β-keto group is reduced to a hydroxyl group. If a module is a minimal module plus a KR and DH, then the affected β-keto group is first reduced to a hydroxyl and then dehydrated to a double bond between the β-carbon of the previously added two-carbon unit and the α-carbon of the just-added two-carbon unit. For example, if the β-carbon is C-11, then the resulting double bond is between C-10 and C-11. If a module is a minimal module plus a KR, DH and an ER, then the β-keto group is fully reduced to a —CH₂—.

In the tylactone PKS, modules 2 and 3 each have a KR and a DH and so result in a double bond at the appropriate positions. So if a hydroxyl group at C-13 (and/or C-15) is desired instead of the double bond between C-12 and C-13 (and/or between C-10 and C-11) as found in tylactone, this can be achieved, for example, by inactivating or deleting the DH domain in module 2 (and/or module 3) of the tylactone PKS enzyme. Similarly, if a hydroxyl group at C-9 is desired instead of the keto group, this can be achieved for example, by mutating the inactive KR so that it becomes active, or by adding an active KR domain into module 4.

As can be seen, a PKS that makes any one of the above-described macrolactones can be altered to make a different macrolactone. An illustrative set of carbon fragments that can be made by modules from naturally occurring PKS enzymes is shown in Table 3. The PKS gene can be altered by modifying coding sequence for the domains in a module or by replacing the coding sequence for the entire module with that for another module that contains the desired AT and reductive cycle domains.

TABLE 3

| Domains in | | | | | (R = Et, OH, |
|---|---|---|---|---|---|
| addition to | | methyl-malonate | | R— malonate | etc.) |
| minimal module | malonate | (R)—CH₃ | (S)—CH₃ | (R)—R | (S)—R |
| none | 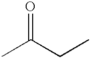 | 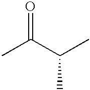 | 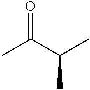 | 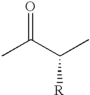 | 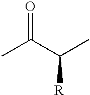 |
| (R)-KR | 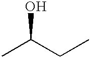 | 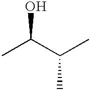 | 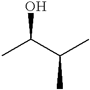 | 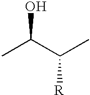 | 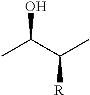 |
| (S)-KR | 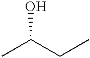 | 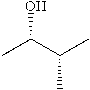 | 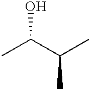 | 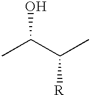 | 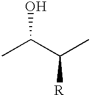 |
| DH/KR | 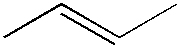 | 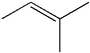 | | 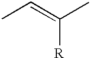 | |
| DH/ER/KR | 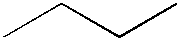 | 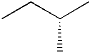 | 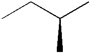 | 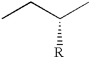 | 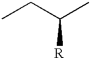 |

As shown in Table 3, the methyl side chain, the R-side chain, and the β-hydroxyl group can be either stereoisomer. ACP domains are believed to bind (S)-methyl-malonyl or (S)-R-malonyl extender units selectively and this configuration is retained unless an epimerase activity converts the side chain to the opposite configuration. To replace a side chain at a particular carbon position in a macrolactone, one can, in addition to altering the AT and/or reductive cycle domains, change the entire module.

Another method for making a macrolactone is chemobiosynthesis, a method in which a synthetic starter unit is incorporated in the biosynthesis of a macrolactone. In this method, a PKS enzyme is unable to process one of its natural substrates due to a mutation in a KS domain that is employed. In one embodiment, the mutation is a KS1 null mutation, an inactivating mutation in the ketosynthase domain of the first extender module that prevents the propagation of the starter unit and permits the introduction of exogenous synthetic thioesters into the C-14, C-15, and C-16 positions of the macrolactone. See U.S. Pat. Nos. 6,066,721 and 6,060,555 and PCT publications WO 99/03986 and WO 97/02358, which are each incorporated herein by reference.

The present invention provides novel compounds useful in chemobiosynthesis of sixteen-membered macrolacones. In one aspect of the present invention, an N-acyl-cysteamine thioester of the formula

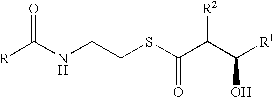

is provided where R, R¹ and R² are each independently hydrogen, aliphatic, aryl, or alkylaryl. In one embodiment, R is C₁-C₅ alkyl; R¹ and R² are each independently hydrogen, C₃-C₁₀ alkyl, C₂-C₁₀ alkenyl, C₂-C₁₀ alkynyl, C₁-C₁₀ haloalkyl, C₁-C₁₀ hydroxyalkyl; C₁-C₁₀ or azidoalkyl. In another embodiment, R is C₁-C₅ alkyl; R¹ is C₃-C₅ alkyl, C₂-C₅ alkenyl, C₁-C₃ haloalkyl, C₁-C₅ hydroxyalkyl; C₁-C₅ or azidoalky; and R² is hydrogen or methyl. In another embodiment, R is methyl or ethyl; R¹ is allyl, azidoethyl, azidomethyl, butenyl, butyl, chloroethyl, chloromethyl, ethyl, fluoroethyl, fluoromethyl, methyl, propyl, and vinyl. In another embodiment, R is ethyl; R¹ is azidoethyl, azidomethyl, butenyl, fluoroethyl, and fluoromethyl; and R² is hydrogen. In another embodiment, the N-acyl-cysteamine thioester is of the formula

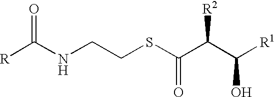

where R is ethyl; R¹ is azidoethyl, azidomethyl, butenyl, fluoroethyl, and fluoromethyl; and R² is methyl. In another embodiment, the N-acyl-cysteamine thioester is of the formula

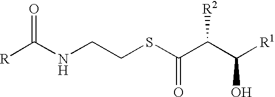

where R is ethyl; R¹ is azidoethyl, azidomethyl, butenyl, fluoroethyl, and fluoromethyl; and R² is methyl.

In another aspect of the present invention, methods for making synthetic thioesters of the formula

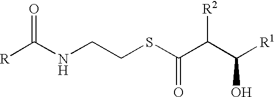

are provided where R is acyl; R¹ is an aliphatic; and R² is hydrogen. One embodiment for making such thioesters is illustrated by Scheme 3.

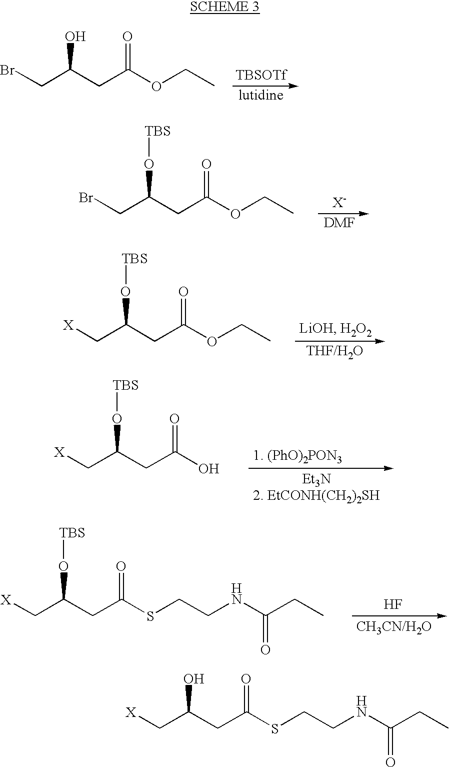

Commercially available ethyl-(3S)-4-bromo-3-hydroxybutyrate is silylated and subject to nucleophilic displacement with nucleophile X⁻. The resulting product is hydrolyzed, treated with diphenyl phosphorylazide and then N-propionylcysteamine. The protected thioester is then desilyated to yield the desired synthetic thioester for use in the present invention. Examples 9 and 10 further illustrate this embodiment where X⁻ is N₃⁻.

In another aspect of the present invention, methods for making synthetic thioesters of the formula

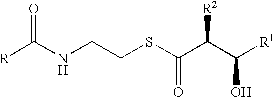

are provided where R² and the hydroxyl are in a syn-configuration and R is acyl and R¹ and R² are each independently substituted or unsubstituted aliphatic. Scheme 4 illustrates one embodiment for making these thioesters.

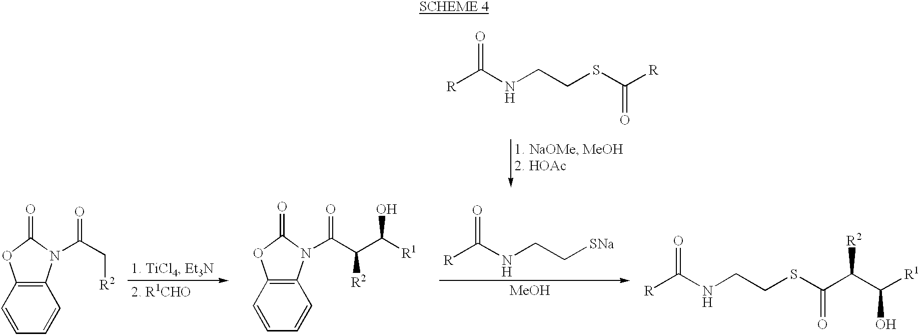

An N-acyl-2-benzoxazolone is subject to a syn-selective aldol condensation by treating the chiral auxiliary with titanium tetrachloride and triethylamine. Thioesterification of the resulting syn-product leads to the desired N-acyl-cysteamine thioester. Other synthetic methods and illustrative examples of N-acyl-cysteamine thioesters can that be synthesized are described by, for example, PCT Publication WO 00/44717 which is incorporated herein by reference.

In another aspect of the present invention, methods for making synthetic thioesters of the formula

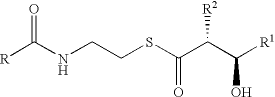

are provided where R² and the hydroxyl are in an anti-configuration and R is acyl and R¹ and R² are each independently substituted or unsubstituted aliphatic. Scheme 5 illustrates one embodiment for making these thioesters.

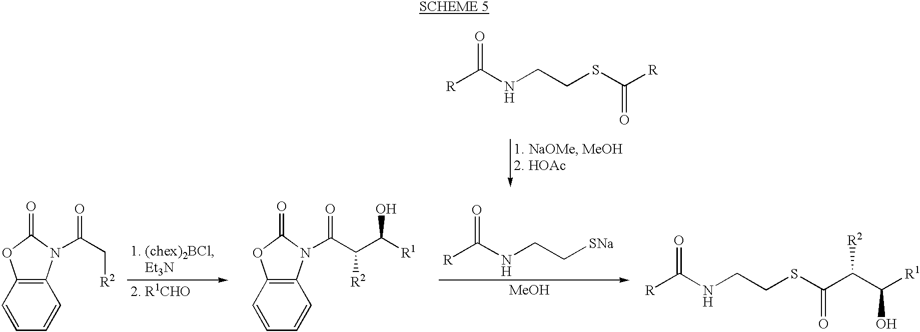

The method of Scheme 5 is similar to that of Scheme 4 except that chlorodicyclohexylborane is used so that the aldol condensation is anti-selective. See Evans et al., Tetrahedron 48: 2127-2142 (1992), which is incorporated herein by reference. Examples 1-15 describe the synthesis of specific embodiments of the above-described thioesters and the intermediates thereto.

In another aspect of the present invention, methods of making a macrolactone from a synthetic thioester of the formula

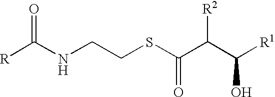

are provided (where R, R¹, and R² are as previously described). A thioester is provided to a PKS that makes a sixteen-membered macrolactone and has a KS1 null mutation, to produce a macrolactone containing:

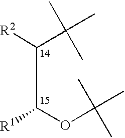

where R² and R¹ are as described above. In another embodiment, a racemic thioester is provided to a PKS that makes a sixteen-membered macrolactone and has a KS1 null mutation to produce a macrolactone containing:

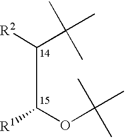

where R² and R¹ are as described above.

The side chain introduced at C-14 and/or C-15 can be selected to modulate the hydrophilic or electronegative character of the macrolactone compound. For example, the present invention provides macrolactones containing a fluoroalkyl side chain at C-15. As illustrated in Scheme 6, (3S)-5-fluoro-3-hydroxypentanoate N-propionylcysteamine thioester can be used to obtain such macrolactones from either a platentolide PKS or a tylactone PKS containing a KS1 null mutation.

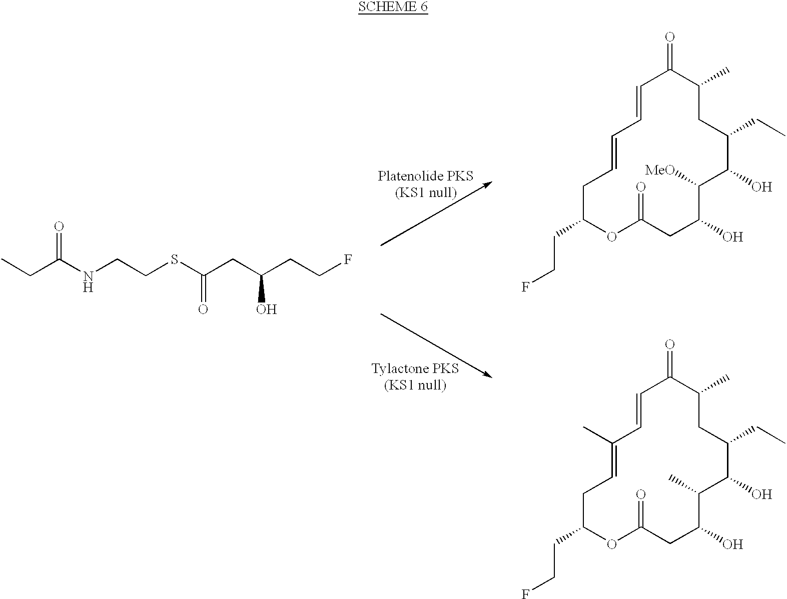

The modified product from the platenolide PKS has the platenolide skeleton but for the group at C-15. The modified product from the tylactone PKS is a tylactone but for the hydrogen instead of a methyl at C-14 and a fluoroethyl instead of an ethyl at C-15.

In other embodiments, the side chain introduced at C-14 and/or C-15 includes a chemical handle for subsequent transformations. For example, the present invention provides a macrolactone with an azidoalkyl side chain, which can be chemically converted to an aminoalkyl side chain, which can be further modified to provide additional compounds of the invention. As shown in Scheme 7, either diastereomer of 4-azido-3-hydroxy-2-methylbutyrate N-propionylcysteamine can be used to obtain an azido-modified PKS product by chemobiosynthesis.

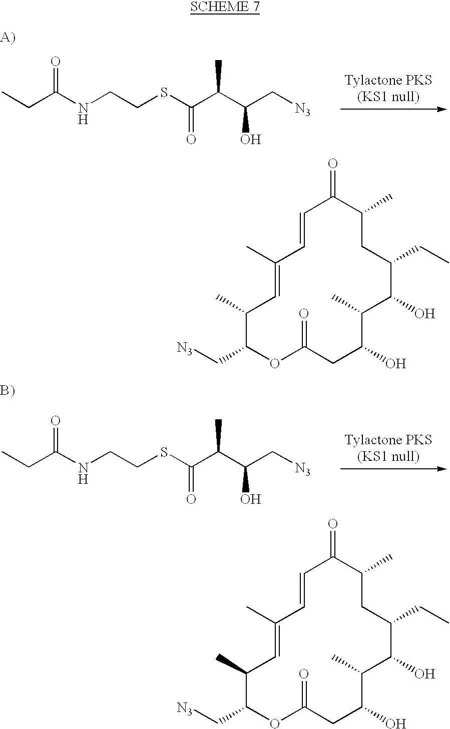

When (2S,3S)-4-azido-3-hydroxy-2-methylbutyrate N-propionylcysteamine is used with a tylactone PKS (Scheme 7A), a modified tylactone having a methyl of the opposite stereochemistry at C-14 and an azidomethyl at C-15 is obtained. When (2S,3S)-4-azido-3-hydroxy-2-methylbutyrate N-propionylcysteamine is used, a modified tylactone having an azidomethyl at C-15 is obtained (the methyl at C-14 is of the same stereochemistry as that found in tylactone). In either situation, the azido is converted into an amine in certain embodiments. In other embodiments, the resulting amine is further modified to provide additional compounds of the invention.

Post-PKS Modifications

After the macrolactone is made, a number of compounds of the present invention can be made by post-PKS modifications. These modifications include alterations in the oxidation state of groups off the macrolactone (e.g., with hydroxylases, dehydratases, epoxidases and the like); addition of methyl and/or hydroxyl (e.g., with methyl transferases and hydroxylases); and addition of saccharides (e.g., with glycosyltransferases). The genes for naturally-occurring post-PKS modification enzymes as well as for the biosynthesis of accessory products such as saccharides are often contiguous with the genes for the PKS and are part of the macrolide biosynthetic gene cluster. As described in greater detail below, the post-PKS modifications that occur in nature can be altered by deleting genes having unwanted functionalities (or by inhibiting the gene product's activities) and/or adding genes having the desired functionalities not normally present into the natural cluster. These changes can be made independently or in conjunction with modifications to the PKS gene. In addition, the resulting product can be further modified using biochemical and/or synthetic methods.

To illustrate, the post-PKS modifications to tylactone are described in Scheme 2. The activities of these modification enzymes can be eliminated to make compounds that differ from tylosin at a number of positions. An illustrative example is TylI, which oxidizes the ethyl side chain at C-6 into methyl aldehyde. Because the resulting aldehyde is not involved in subsequent modifications, eliminating TylI activity does affect the function of the other enzymes in the pathway and results in a tylosin derivative having an ethyl instead of a methyl aldehyde at C-6. In contrast, TylH oxidizes the methyl side chain at C-14 into hydroxymethyl to which deoxyallose subsequently is added and converted to mycinose. Because the resulting alcohol is involved in subsequent glycosylation and modification steps, eliminating the function of TylI results in the de facto elimination of other modification enzyme functions (TylD, TylJ, Tyl E and Tyl F) and results in a tylosin derivative having a methyl at C-14 instead of an —O-methyl-mycinose at this position.

Random mutagenesis with a mutating agent such as UV light can be used to generate mutants where one or more of the tailoring or modification enzymes are inactivated. In fact, many derivatives of sixteen-membered macrolide antibiotics are made by naturally occurring mutants. One example of such a compound is demycinosyltylosin (“DMT”) whose structure is shown below

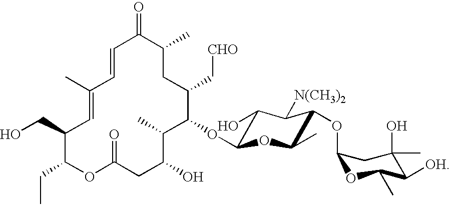

This compound was isolated from a mutant strain of S. fradiae (NRRL 12170) that is unable to make 6-deoxyallose or attach the sugar moiety to the hydroxymethyl group at C-14. See U.S. Pat. No. 4,321,361 (which is incorporated herein). This compound can also be made using recombinant methods by introducing an inactivating mutation in genes involved in the biosynthesis of 6-deoxyallose (tyl J & tyl D) or the gene involved in the addition of 6-deoxyallose to O-mycaminosyl-tylonolide (6, see Scheme 2).

One can add enzymatic activities to a cell in accordance with the present invention. For example an epoxidase not normally present in the tylosin biosynthetic gene cluster can be added to a tylosin-producing cell to make 12,13-epoxy-tylosin whose structure is shown below

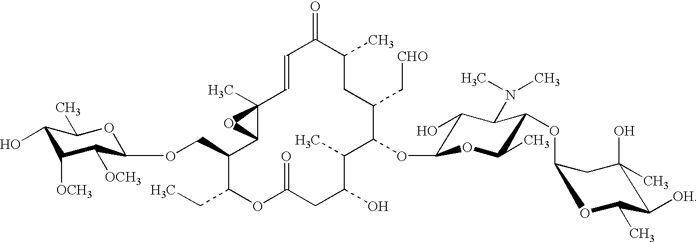

Any suitable epoxidase gene can be used including, for example, the epoxidase gene from the angolamycin, carbomycin or rosamicin PKS cluster that epoxidates the macrolactone at a similar position in angolamycin or carbomycin.

One can also alter the sugar moieties to add or change sugars attached to the macrolactone in accordance with the present invention. The genes required for biosynthesis and attachment of the sugar moieties to the macrolide are typically found as part of the macrolide biosynthetic gene cluster. These genes can be isolated from one cell and inserted into another. Illustrative examples of amino sugars that can be added include: D-desosamine; D-mycaminose; D-angolasamine; D-forosamine; and L-megosamine. Illustrative examples of neutral sugars that can be added include: D-chalcose; D-aldgarose; D-mycinose; 4,6-dideoxy-D-threo-hexos-3-ulose; 6-deoxy-2-0-methyl-D-allose; L-mycarose; L-cladinose; L-oleandrose; L-cinerulose A; L-arcanose; and 3-methyl-2,3,6-trideoxy-L-thre-hex-2-enopyranose.

In tylosin, the sugar moieties are mycaminose and mycarose that are at the C-5 hydroxyl as a disaccharide, and mycinose which is attached to the hydroxymethyl at C-14. In accordance with the present invention, any combination of these sugars can be deleted and/or replaced with another sugar. One example of such compounds is 5-O-desosaminyl-tylactone whose structure is shown below

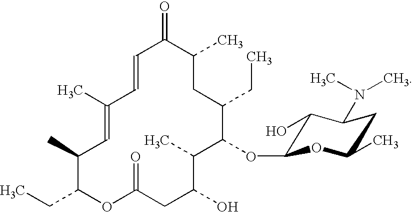

This compound can be made, for example, by inactivating all of the existing tailoring enzymes in the tylosin biosynthetic gene cluster and replacing them with the genes necessary for making and attaching desosamine. The desosamine genes include those, for example, in the pikromycin biosynthetic gene cluster.

Another example is 23-O-des(mycinosyloxy)-9-dihydro-9-O-forosaminyl tylosin whose structure is shown below

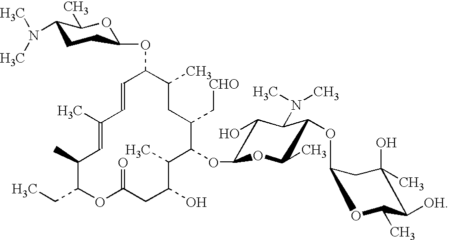

This compound can be made, for example by inactivating TylH which hydroxylates the C-14 methyl (thereby preventing the attachment of the deoxyallose) and by adding the genes necessary for making and attaching forosamine as well as the gene responsible for reducing the C-9 ketone. These genes can be isolated from, for example, the spiramycin biosynthetic gene cluster.

Bioconversion can also be used to make compounds of the present invention. In the bioconversion process, a macrolactone or other compound is provided to an organism that can convert the macrolactone or other compound to a different compound. For example, this method has been used to make both 5-O-desosaminyl-tylactone and 23-O-des(mycinosyloxy)-9-dihydro-9-O-forosaminyl tylosin. See Omura et al., J. Antibiot. 33: 1570 (1980) and Omura et al., J. Antibiot. 36: 927 (1983), which are each incorporated herein by reference. Tylactone was isolated from a mutant strain of S. fradiae (KA-427-261) that was unable to further process the PKS product. The synthesis of the PKS product of the second organism was inhibited by growing the organism in the presence of cerulenin.

5-O-desosaminyl-tylactone was made by adding tylactone to a pikromycin producing strain in the presence of cerulenin. The strain substituted tylactone for pikronolide and added a desosamine to the C-5 hydroxyl of tylactone. 3-O-desmycinosyl-9-desoxo-9-O-forosaminyl tylosin was made in a similar manner. Tylactone was added to a spiramycin producing S. ambofaciens that was grown in the presence of cerulenin. The strain substituted tylactone for platenolide, and added 4-mycinosyl-mycaminose at C-5, reduced the C-9 ketone and added forosamine at the resulting C-9 alcohol.

The above described methods can be used in combination to make additional compounds of the present invention. For example, a platenolide-based macrolactone having a fluoroethyl or a vinyl group at C-15 can be made using chemobiosynthesis. This macrolactone can then be added to a tylosin producing strain grown in the presence of cerulenin to yield a tylosin derivative having a fluoroethyl or a vinyl at C-15. Replacing the S. fradiae strain with any of the strains listed in Table 2 as the bioconversion strain would yield the corresponding macrolide derivative having a fluoroethyl or vinyl at C-15. In other embodiments, the strain used for bioconversion is a recombinant host where one or more of the modification or tailoring enzymes are inactivated. For example, a strain of S. fradiae can be made in which the tylG and tylI are inactivated. Scheme 8 illustrates the macrolides that are made when a 15-R platenolide (where R is for example vinyl or fluoroethyl) is added to such a strain grown in the presence of cerulenin.

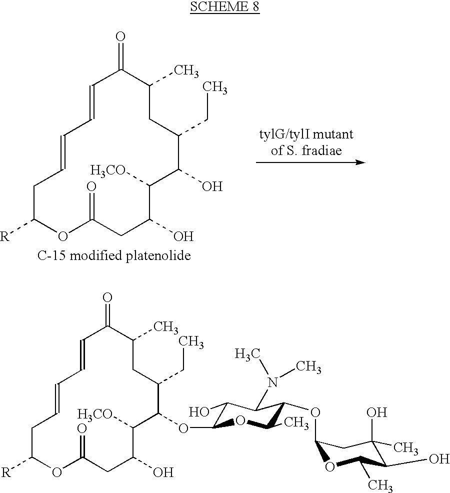

Chemical Modifications

The macrolactones of the present invention can be further modified using chemical synthesis. For example, a sixteen-membered macrolide with an —CH₂CHO at C-6 (e.g. carbomycin A, deltamycins, leukomycins, midecamycins, platenomycins and tylosin) can be deformylated at C-19 by treatment with Wilkinson's catalyst tris(triphenylphosphine)rhodium chloride) in refluxing benzene) to result in the corresponding compound having a C-6 methyl instead. See also U.S. Pat. No. 4,345,069 which is incorporated herein by reference. Scheme 9 illustrates this reaction as it is applied to DMT to make 19-formyl-DMT.

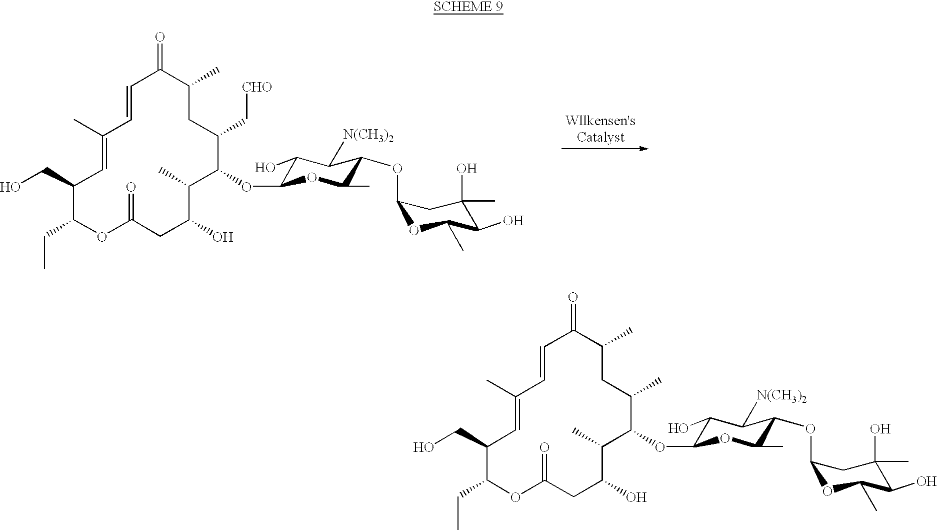

DMT can be obtained, for example, from a mutant strain of S. fradiae (NRRL 12170; U.S. Pat. No. 4,321,361).

Another example is the formation of a sixteen-membered ketolide where a C-3 hydroxyl of a macrolide is oxidized to a ketone. In one method, a 3-acyl-containing macrolide is deacylated. In another method, a suitably protected 3-hydroxy-macrolide is oxidized to a ketone using a modified Swern oxidiation procedure. In this procedure, an oxidizing agent such as N-chlorosuccinimide-dimethyl sulfide or a carbondiimide-dimethylsulfoxide is used.

Recombinant Methods

Methods for altering genes found in the macrolide biosynthetic gene cluster and expressing them in hosts cells are described for example by U.S. Pat. Nos. 5,672,491; 5,830,750; 5,843,718; 5,712,146; 5,962,290; 6,022,731; 6,066,721; 6,077,696; 6,080,555; 6,215,007; 6,214,573; and 6,221,641 which are each incorporated herein by reference. In general, a gene (or genes) of interest is cloned into one or more plasmids in Escherichia coli so that the gene is more easily manipulated. Once the desired changes to the gene are made, then the portion(s) of the gene containing the changes are reintroduced to the host cell. In certain embodiments where a small number of base changes are introduce (for example, to inactivate various domains), these changes are made using for example QuikChange Kit from Stratagene. In other embodiments where full domains are removed or exchanged, the appropriate sequences are cloned using PCR and appropriate restriction sites are introduced to allow the deletion or replacement of fragments. Once the changes are made (typically in E. Coli), the altered segment is returned to the chromosome of the host using homologous recombination. This is typically accomplished by the cloning of sequences up to about 2 kb in length that flank the altered PKS sequence in the E. coli delivery vectors carrying a selectable genetic marker (e.g. antibiotic resistance). The vectors are then introduced into the host and the first homologous crossover of the two-step recombination cycle is selected. In addition to the plasmid sequences, the segment of the PKS around the crossover site carries duplications of the flanking sequences, a copy of the unaltered PKS sequence and a copy of the altered PKS sequence. Strains that have undergone the second crossover step are found by following the loss of the genetic marker contained in the plasmid. PCR analysis is used to check the genotype of the isolated colonies.

The modification and expression of genes in the macrolide biosynthetic gene cluster preferably occurs in a host cell that does not express genes in another Macrolide biosynthetic gene cluster. Most typically, the genes in the native PKS gene cluster are deleted or otherwise rendered non-functional by mutagenesis or other methods. Such host cells are referred to as clean hosts and general methods for making such cells are described in U.S. Pat. No. 5,830,750 which is incorporated herein by reference. In one embodiment, the clean host is Streptomyces coelicolor (as described by the '750 patent). In another embodiment, the clean host is Streptomyces hygroscopicus var. ascomyceticus (ATCC 14891) which normally makes FK-520. Because this strain of S. hygroscopicus already possesses a methoxy malonyl and an ethyl malonyl pathways, it can be used to make macrolactones that have methoxy and/or ethyl at the even numbered positions. Other examples of suitable host cells include E. coli, Saccharomyces cerevasiae and Myxococcus xanthus. Methods for using E. coli and S. cerevesiae to make polyketides are described, for example, by PCT Publication Nos. WO 01/27306 and WO 01/31035, and by U.S. Pat. No. 6,258,566 which are each incorporated herein by reference. Methods for using M. xanthus as a host cell is described by PCT publication No. WO 01/31247 which is incorporated herein by reference. In certain host cells that do not already express genes necessary for desired modification and tailoring enzymes (and any requisite precursor pathways), these genes can be added. In other embodiments, host cells lacking modification and tailoring enzymes are used such to make the macrolactone and bioconversion methods used to add the desired post-modifications.

In another embodiments, the clean host is a cell that makes a sixteen-membered macrolides in nature. In one embodiment, the clean host is a cell whose PKS normally makes a platenolide. Such cells are particularly versatile because they already include pathways to malonyl CoA, methyl malonyl CoA, ethyl malonyl CoA, and a methoxy malonyl CoA. Illustrative examples include any one of the Type II macrolactone hosts listed in Table 2 such as S. ambofaciens; S. caelestis; S. djakartensis; S. deltae; S. fungicidus; S. halstedii; S. hygroscopicus (maridomycin producer); Stv. kitosatoensis; S. mycarofaciens; S. narbonensis; S. platensis; and S. thermotolerans. Depending on the particular host, post-PKS genes such as those for epoxidases, hydroxylases, glycosidases are included as well as the necessary genes for forosamine, mycaminose, mycarose, and mycinose.

In another embodiment, the clean host is a cell whose PKS normally makes a tylactone. These cells include pathways to malonyl CoA, methyl malonyl CoA, and ethylmalonyl CoA. Illustrative examples include but are not limited to any one of the Type I hosts in Table 2 such as M. capillata; M. chalcea var. izumensis; Mrosaria; S. cirratus; S. eurythermus; S. hygroscopicus (relomycin producer); S. flocculus; Streptomyces sp. SK-62; and S. fradiae. Depending on the particular host, post-PKS genes such as those for epoxidases, hydroxylases, glycosidases are included as well as the necessary genes for desosamine, mycaminose, mycarose and mycinose.

In another embodiment, the clean host is a cell whose PKS normally makes a lactone skeleton of Type III. An illustrative example is M. chalcea var. izumensis. Because this host also makes a Type I lactone skeleton, it is possible, that the Type III skeleton is a minor product that results from the occasional loading of a methyl malonyl CoA instead of an ethyl malonyl CoA. This host also includes post-PKS genes for an epoxidase, hydroxylase and a glycosidase as well as the necessary genes for desosamine.

In another embodiment, the clean host is a cell whose PKS normally makes a lactone skeleton of Type IV. An illustrative example is M. griseorbida. This host also includes post-PKS genes for an epoxidase and glycosidases as well as the necessary genes for mycinose and desosamine.

In another embodiment, the clean host is a cell whose PKS normally makes a lactone skeleton of Type V. Illustrative examples include but are not limited to S. albogriseolus and S. lavendulae. Depending on the particular host, post-PKS genes such as those for an epoxidase, hydroxylase, and glycosidases are included as well as the necessary genes for aldgarose, chalcose, and mycinose.

In another embodiment, the clean host is a cell whose PKS normally makes a lactone skeleton of Type VI. An illustrative example is S. rimosus. This host includes post-PKS genes for an epoxidase, hydroxylase and glycosidases as well as the necessary genes for chalcose and mycinose.

Synthetic Methods

In one aspect of the present invention, methods are provided for adding side chains Z and Z′. The synthetic methods that follow rely in part on standard protocols which are found in, for example, Advanced Organic Chemistry 3rd Ed. by Jerry March (1985) which is incorporated herein by reference.

General Considerations

Free hydroxyl groups, particularly those found in saccharides, in the starting macrolide compound may need to be protected before commencing any of the following transformations. A variety of suitable protecting groups are disclosed, for example, in T. H. Greene and P. G. M. Wuts, Protective Groups in Organic Synthesis, Third Edition, John Wiley & Sons, New York (1999). In certain embodiments, the starting macrolide compounds include saccharides whose free hydroxyls generally need to be protected during one or more chemical transformations. Conversion of one or more of these free hydroxyls into other groups has been shown to enhance the bioavailability and/or potency of the compound as an antibiotic. For example, many of the starting macrolide compounds include a substituted or unsubstituted 4-mycarosyl-mycaminose off the C-5 hydroxyl. Modification of the 3′-hydroxyl has been shown to affect bioavailability and modification of the 4′-hydroxyl has been shown to affect PT activity. Consequently, the moieties that are used to protect these hydroxyl groups can also serve a dual purpose. In one embodiment, the 2-hydroxyl of mycaminose and the 3′-hydroxyl of mycarose are each protected with acetyl groups which can be readily removed with methanol, and the 4′-hydroxyl is protected with a group selected from the group consisting of a non-acetyl acyl group or an aromatic containing group. In another embodiment, the 2-hydroxyl of mycaminose is protected with an acetyl group which is later removed with methanol; 3′-hydroxyl of mycarose is protected with a group selected from acetyl, propionyl, butyryl, and isovaleryl; and the 4′-hydroxyl of mycarose is protected with a group selected from propionyl, butryl, isovaleryl or an aromatic protecting group. The 3′-protecting group can remain or be removed. Illustrative examples of suitable 4′-hydroxyl groups include but are not limited to: isovaleryl; phenylacetyl; phenylthioacetyl; phenylsulfonylacetyl; 4-nitrophenylacetyl; 4-nitrophenylsulfonyl; and phenylethanesulfonyl.

Scheme 10 outlines one protection strategy for 4-mycarosyl-mycaminose where the 3′ and 4′ protecting groups optionally can remain as part of the final compound.

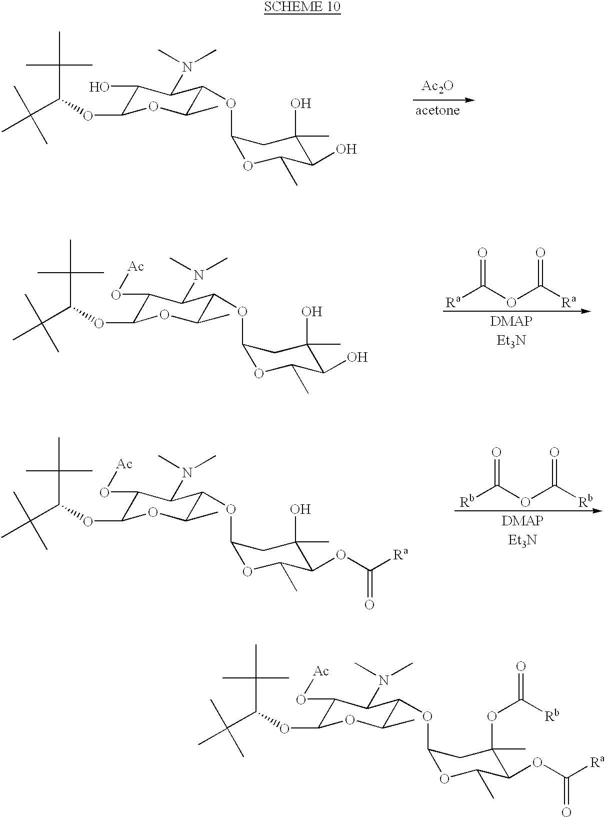

The acylation reactions are selective because of the differences in reactivities of the free hydroxyls. First, the 2′-hydroxyl of the macrolide is acetylated. Next, the 4″-hydroxyl is acylated. If the 3″-hydroxyl is to be protected using the same group as the 4″-hydroxyl, then longer reaction times can be used. Note that R^{a} and R^{b} are each independently aliphatic, aryl, or alkylaryl. If a different group is desired, then the reaction can be stopped when the 4″-hydroxyl is protected and a different acyl group can be used to protect the 3″-hydroxyl. If the 3″-hydroxyl is to remain unprotected in the final compound, it can also be protected using a acetyl group or another group which can readily be removed.

Macrolides Possessing a Side Chain Z

In one aspect of the present invention, methods for making sixteen-membered macrolide possessing a side chain Z are provided where Z is aliphatic, aryl, alkylaryl, halide, ═NOR^{c}, ═NNHR^{c}, or —W—R^{c} where W is O, S, NC(═O)R^{d}NC(═O)OR^{d}NC(═O)NHR^{d} or NR^{d} where R^{c} and R^{d} are each independently hydrogen, aliphatic, aryl or alkylaryl. In many cases, the starting materials for these compounds are macrolides that possess a hydroxyl at the carbon to which Z is attached.

In one embodiment, Z is attached to one of the following positions of a sixteen-membered macrolide: C-7, C-11, and C-13. In one method, the starting compounds are novel macrolides of the invention that are include derived from recombinant PKS products where a KR was added, a DH deleted, or a DH and an ER were deleted at the appropriate module of the PKS gene. In another method, the starting compounds are 7-hydroxy-macrolides that are made chemically starting from 6,7-dehydro macrolides. Both the 6,7-dehydro macrolides and 7-hydroxy-macrolides are novel compounds of the invention. In one embodiment of this chemical conversion, the starting material is a 6,7-dehydro-9-hydroxy macrolide that is oxidized with an oxidizing agent such as manganese dioxide to a 6,7-dehydro-9-oxo-macrolide. In another embodiment, starting material is the 6,7-dehydro-9-oxo macrolide. Scheme 11A illustrates one method of chemically obtaining a 7-hydroxy macrolide from a 6,7-dehydro macrolide.

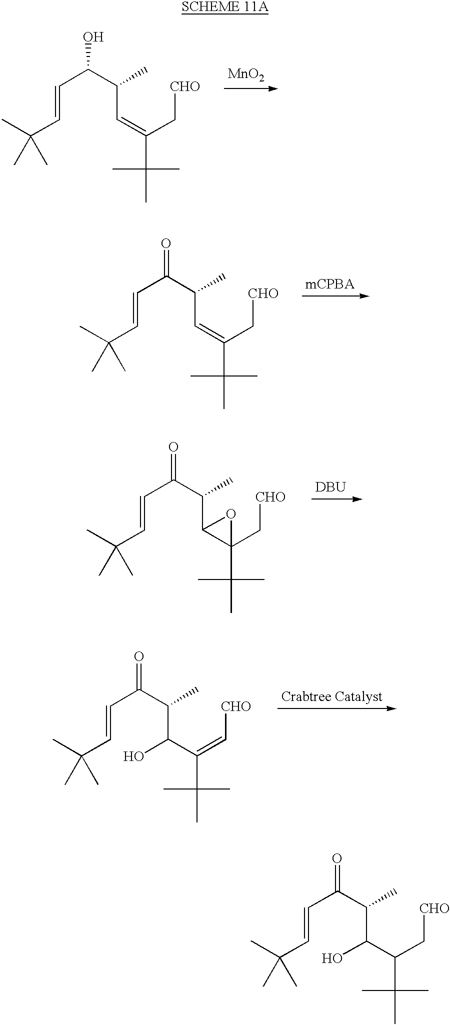

The double bond between C-6 and C-7 is epoxidized using for example, meta-chloroperbenzoic acid. The resulting epoxide is eliminated using for example, 1,8-diazabicyclo[3.7.0]octane to yield the 6-enal-7-hydroxy macrolide. The 6-enal moiety can be selectively reduced using the methods of the present invention. In one method, the 6-enal moiety is reduced using for example copper hydride. In another method, the 6-enal moiety is reduced using hydroxyl-directed hydrogenation using for example, a metal catalyst such as the Crabtree catalyst.

Scheme 11B illustrates another method of chemically obtaining a 7-hydroxy macrolide from a 6,7-dehydro macrolide.

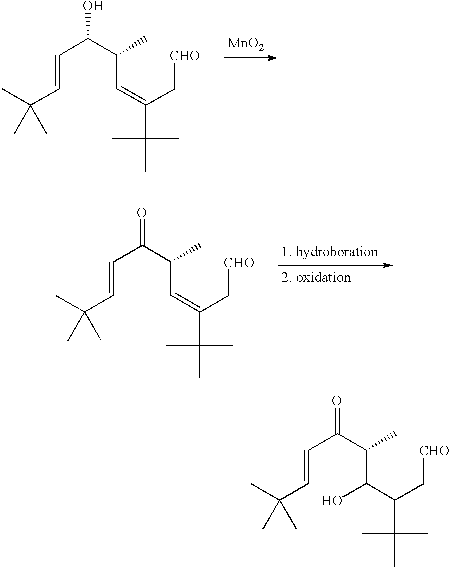

In one embodiment, the starting material is a 6,7-dehydro-9-hydroxy macrolide that is oxidized with an oxidizing agent such as manganese dioxide to a 6,7-dehydro-9-oxo-macrolide. In another embodiment, starting material is the 6,7-dehydro-9-oxo macrolide. The 6,7-dehydro-9-oxo macrolide is hydroborated and oxidized (e.g., H₂O₂) to yield the 7-hydroxy-9-oxo macrolide.

In another embodiment, Z is attached to C-8 of a sixteen-membered macrolide. The starting compound is a 8-hydroxy sixteen-membered macrolide, a novel compound of the invention. In one method, 8-hydroxy macrolide is obtained by hydroxylating a starting macrolide compound at C-8 using biochemical methods. In one embodiment, a macrolide is converted into a 8-hydroxy-macrolide using an oleandomycin-producing strain (or the oleandomycin epoxidase or another epoxidase) that adds an epoxide at C-8 which subsequently is converted into a hydroxyl group. In another embodiment, a macrolide is converted into a 8-hydroxy-macrolide using a chalcomycin-producing strain (or the chalcomycin hydroxylase or other hydroxylase) that adds a hydroxyl group at C-8. In another method, 8-hydroxy macrolide is obtained by direct chemical hydroxylation. Scheme 12 describes one embodiment of this chemical method.

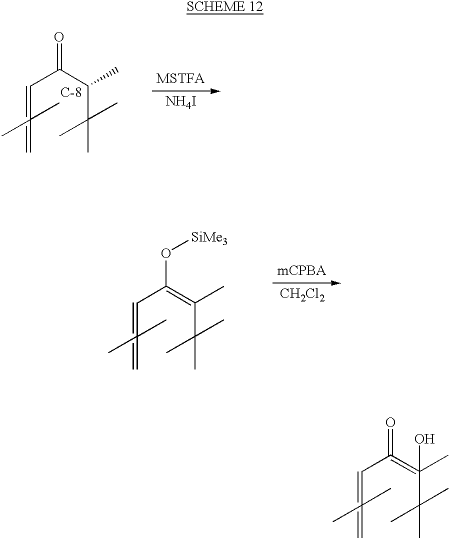

A suitably protected 9-oxo macrolide is converted into a 8,9-silyl enolether. In one method, the 9-oxo-macrolide is converted into a 8,9-silyl enolether using N-methyl-N(trimethylsilyl)-trifluoroacetamide. In another method, the 9-oxo-macrolide is converted into a 8,9-silyl enolether using sodium hexamethyldisilazide and chlorotrimethylsilane. The silyl enolether is treated with meta-chloroperbenzoic acid in a Rubottom oxidation to yield the 8-hydroxy-9-oxo macrolide.

In another embodiment, Z is attached to C-6 of a sixteen-membered macrolide. In yet another embodiment, Z is attached to a vinyl group at C-6 of a sixteen-membered macrolide. The starting materials for these compounds are 6,7-dehydro-macrolides that have been chemically converted into 6-hydroxy-6-vinyl compounds. Scheme 13 describes one method for this transformation.

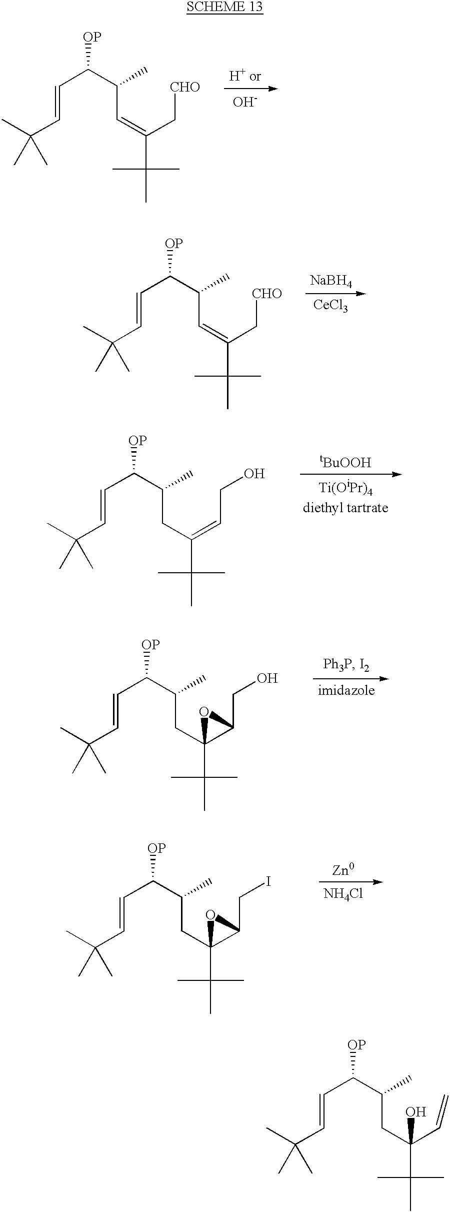

The double bond between C-6 and C-7 of a suitably protected 9-hydroxy-6,7-dehydro macrolide (where P is a hydrogen or a hydroxy protecting group) is isomerized with mild acid or base. The 6-enal is reduced with sodium borohydride to an allylic alcohol and epoxidized in a Sharpless asymmetric epoxidation. The epoxy-alcohol is iodinated and then reduced to yield the 6-hydroxy-6-vinyl product, a novel compound of the invention. In one method, the 6-hydroxy moiety is the attachment point for side chain Z. In another method, the 6-vinyl group is the attachment point for side chain Z. Examples 21-26 describe specific embodiments of this method.

In another embodiment, Z is attached to C-13 of an 11-ene-13-hydroxy macrolide, a novel compound of the invention. In one method, the 11-ene-13-hydroxy macrolide is obtained from a 10, 12-diene-containing macrolide. In another method, the 11-ene-13-hydroxy macrolide is obtained from a 10-en-12,13-epoxy-macrolide. Scheme 14 describes one method for these conversions.

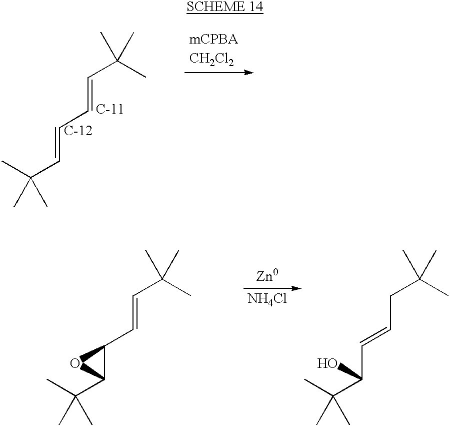

If starting with the diene, it is epoxidized using an epoxidating agent such as meta-chloroperbenzoic acid or triphenylphosphine. The 12,13-epoxide is reduced using a reducing agent such as zinc or samarium diiodide to yield the 11-en-13-hydroxy macrolide.

In another embodiment, Z is attached to C-12 of a 9-oxo-10,12-diene-12-hydroxymethyl-macrolide, a novel compound of the invention. The 19-oxo-10,12-dienyl-12-hydroxymethyl-macrolide is obtained for example by direct hydroxylation using selenium dioxide of a 9-oxo-10,12-dienyl-12-methyl-macrolide.

In another embodiment, Z is attached to either C-12 or C-13 of a 12,13-dihydroxy macrolide, a novel compound of the invention. The 12,13-dihydroxy is obtained from the chemical conversion of a 12,13-epoxy macrolide. Scheme 15A illustrates one method for this conversion.

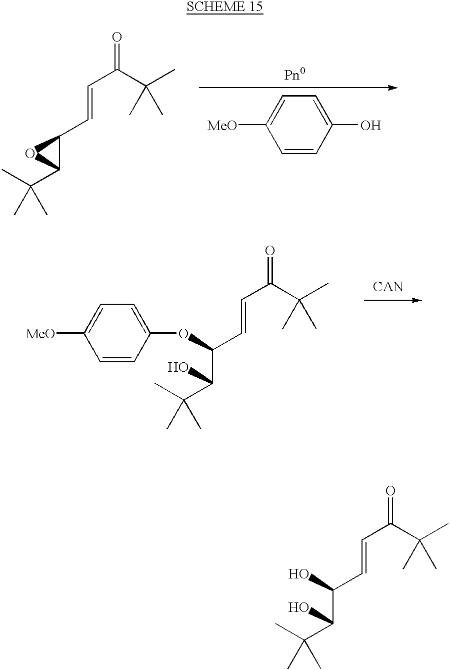

A 12,13-epoxide is treated with a transition metal catalyst (e.g. 1,2-diaminocyclohexane-N,N′-bis (2′-diphenylphosphinobenzoyl) Pd catalyst) and a nucleophile that is capable of acting as a hydroxy protecting group (e.g., para-methoxy-phenol). The paramethoxyphenyl moiety can be removed using for example cerium (IV) ammonium nitrate to expose the free hydroxyl at C-12. In one method, the C-12 hydroxy protected version of the diol is used to make C-13 hydroxy derivatives. In another method, the 12-paramethoxyphenoxy-13-hydroxy macrolide is treated with a hydroxy protecting group (to protect the C-13 hydroxyl) and then treated with cerium (IV) ammonium nitrate. This transformation yields a free hydroxyl group at C-12 and a protected hydroxyl at C-13 which can be further modified at the C-12 position.

In another embodiment Z is attached to a C-3 or C-9 of a sixteen-membered macrolide. The starting materials for these compounds include naturally occurring macrolides such as angolamycin and tylosin which have a C-3 hydroxyl and a C-9 oxo, and leucomycins and maridomycins which have a C-3 hydroxyl and a C-9 hydroxyl. Other starting materials include compounds that are chemically modified such as reduction at C-9 oxo to an alcohol or deacylation at C-3. In one method, the C-3 or C-9 hydroxyl is converted into an acetate moiety and displaced using palladium-mediated nucleophilic displacement with an azide. The azide is then converted into an amine. In one embodiment, the C-3 or C-9 amine is converted into an amide. In another embodiment, the C-3 or C-9 amine is alkylated using an alkylhalide. In another embodiment, the C-3 or C-9 amine is subject to reductive amination conditions by treating it with an aldehyde and sodium cyanoborohydride.

In another embodiment, Z is attached to a C-6 hydroxyethyl. The starting materials for these compounds include naturally occurring macrolides (e.g. spiramycin IV and VI, juvenimicin A4 and relomycin). Other starting materials include compounds that are chemically modified where the C-6-methyl aldehyde is reduced to a C-6 hydroxyethyl.

In another embodiment, Z is attached to a hydroxymethyl at C-14. The starting materials for these compounds include naturally occurring macrolides such as DMT, or compounds derived from chemobiosynthesis or hybrid biosynthesis (e.g., 14-methyl macrolide added to a strain containing a hydroxylase or oxidase).

In another aspect of the present invention, methods are provided for making sixteen-membered macrolides possessing a side chain Z that is an —O-acyl moiety that is obtained from reacting an appropriate free hydroxyl with an acid chloride. In one embodiment, the macrolide possesses a disaccharide that is protected as described in Scheme 10 prior to the acylation reaction. In another embodiment, the macrolide possesses a primary hydroxyl group and a variation in protection strategy is used. One embodiment of such a strategy is described in Scheme 16.

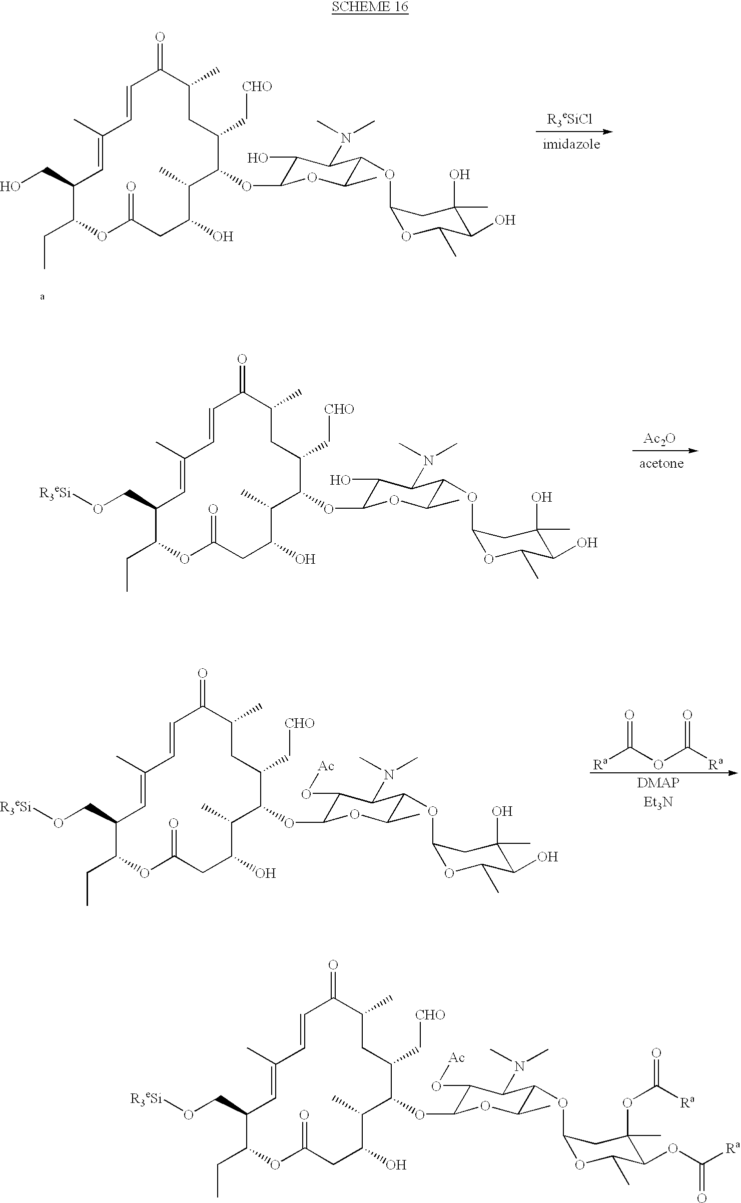

The primary alcohol is silylated with a silating agent containing a bulky group such as t-butyldimethyl silylchloride or R₃^{e}SiCl where R^{e} can be aliphatic, aryl or alkylaryl. Once the primary alcohol is silyated, then the saccharide hydroxyls can be protected as described in Scheme 10. In another embodiment and as shown in Scheme 16, the 2′-hydroxyl is acetylated and the 3″ and 4″ hydroxyls are protected using the same acyl group. Once all of the hydroxyls of the disaccharide are protected, the silyl groups optionally are removed using tetrabutylammonium chloride (Bu₄N⁺F⁻).

In another aspect of the present invention, methods are provided for making sixteen-membered macrolides possessing a side chain Z that is halide or —W—R^{c}, where R^{c} is aliphatic, aryl or alkylaryl and W is O, S, or NR^{d} where R^{d} is hydrogen, aliphatic, aryl or alkylaryl. Scheme 17 illustrates one embodiment of this method with reference to DMT for the purposes of illustration.

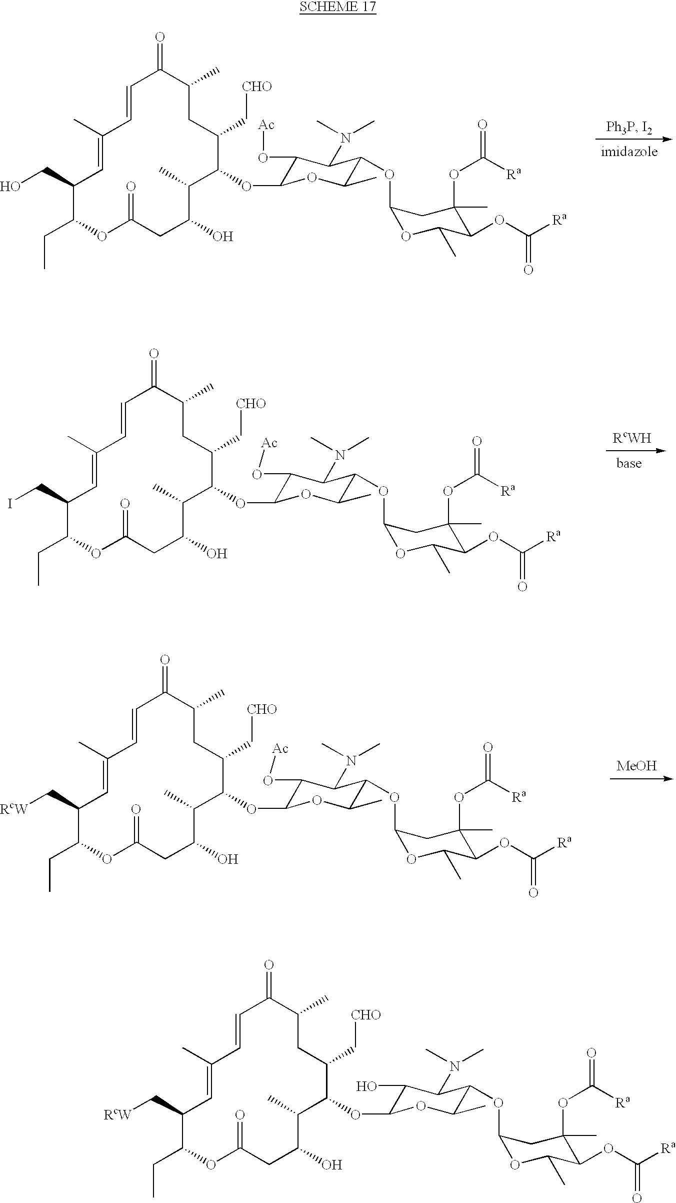

A suitably protected DMT with a free C-14-hydroxymethyl is converted into the corresponding iodomethyl. The iodide is subsequently displaced in a nucleophilic reaction where R^{a} and —W—R^{c} are as previously described. Deprotection using methanol removes the 2′-acetyl group to yield the desired compound. As is the case with any of the compounds of the present invention, they may optionally be further modified. For example, as shown in Scheme 18, the methylaldehyde at C-6 can be converted into an amine (NR^{f}₂ where R^{f} is hydrogen, aliphatic or aryl) which in turn can be further modified.

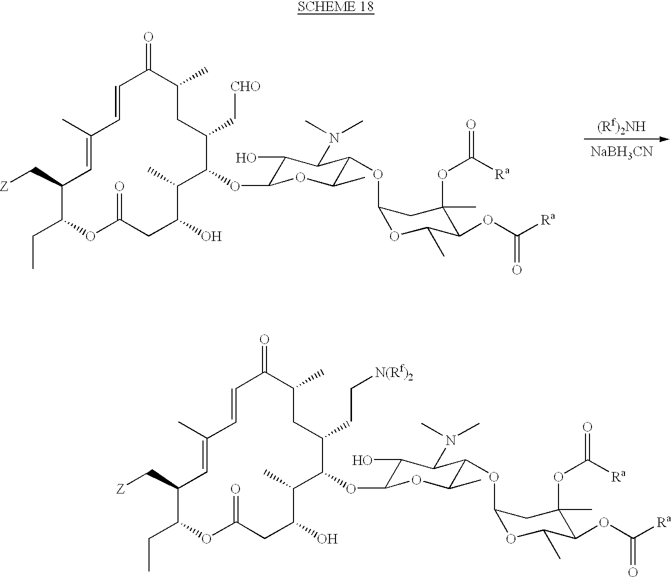

In another aspect of the present invention, methods are provided for making sixteen-membered macrolides possessing a side chain Z that is an O-aliphatic, O-aryl or O-akylaryl. In one embodiment, the appropriate free hydroxyl is reacted with an alkylating agent in the presence of base. Illustrative examples of suitable alkylating agents include alkylhalides and sulfonates such as methyl tosylate, 2-fluoroethyl bromide, cinnamyl bromide, crotonyl bromide allyl bromide, propargyl bromide, and the like. Illustrative examples of suitable bases include potassium hydroxide, sodium hydride, potassium isopropoxide, potassium t-butoxide, and an aprotic solvent. In another embodiment, the appropriate free hydroxyl is alkylated to form an O-aliphatic moiety possessing a terminal double bond that can be further modified by metathesis.

In another embodiment, the appropriate free hydroxyl is alkylated to form an O-aliphatic compound possessing a terminal double bond or a terminal triple bond that may optionally be used in a Heck coupling reaction to form an O-alkylaryl moiety. Scheme 19 illustrates one method with reference to a 19-deformyl-14-hydroxymethyl macrolide for the purposes of illustration.

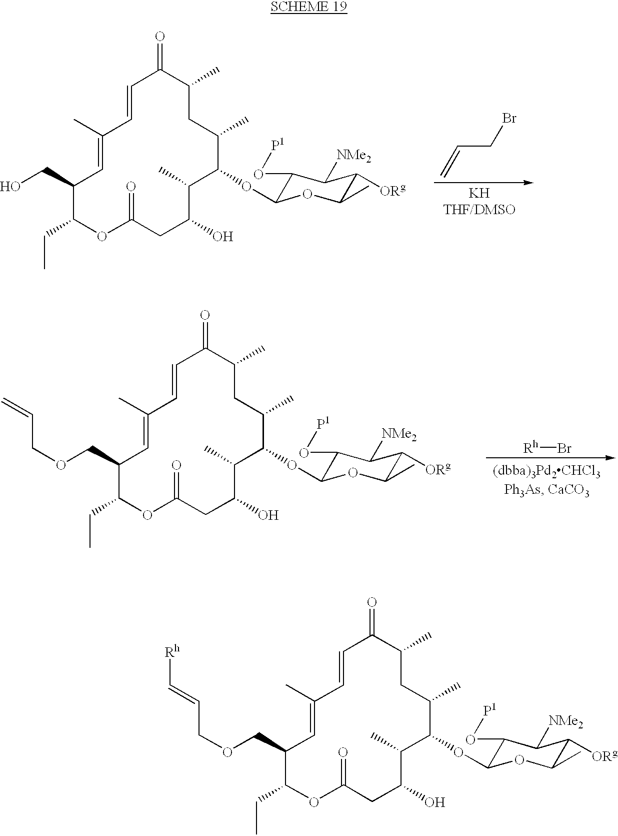

A suitably protected 19-deformyl-14-hydroxymethyl macrolide (where P¹ is a hydroxy protecting group and R^{g} is a hydrogen, hydroxy protecting group or 3″,4″-protected mycarose) is treated with R′X′ where R′ is an aliphatic, aryl or alkylaryl possessing a terminal double or triple bond and X′ is halide, preferably with CH₂═CH—(CH₂)ₙX′ where n is 0-5. In one method and as shown in Scheme 19, R′X′ is allylbromide. In another method, R′X′ is butenylbromide. In yet another method, R′X′ is pentenylbromide. The resulting product is treated with an arylhalide such as R^{h}Br under Heck conditions (Pd(II) or Pd(0), phosphine and amine or inorganic base) and deprotected as desired.

Scheme 20 illustrates another allylation method followed by a Heck coupling reaction with reference to a 19-deformyl macrolide where P¹ and P² are each independently a hydroxy protecting group; R^{g} is a hydroxy protecting group or a 3″,4″-protected mycarose, and Rⁱ is hydroxy protecting group or a 4′″ protected mycinose.

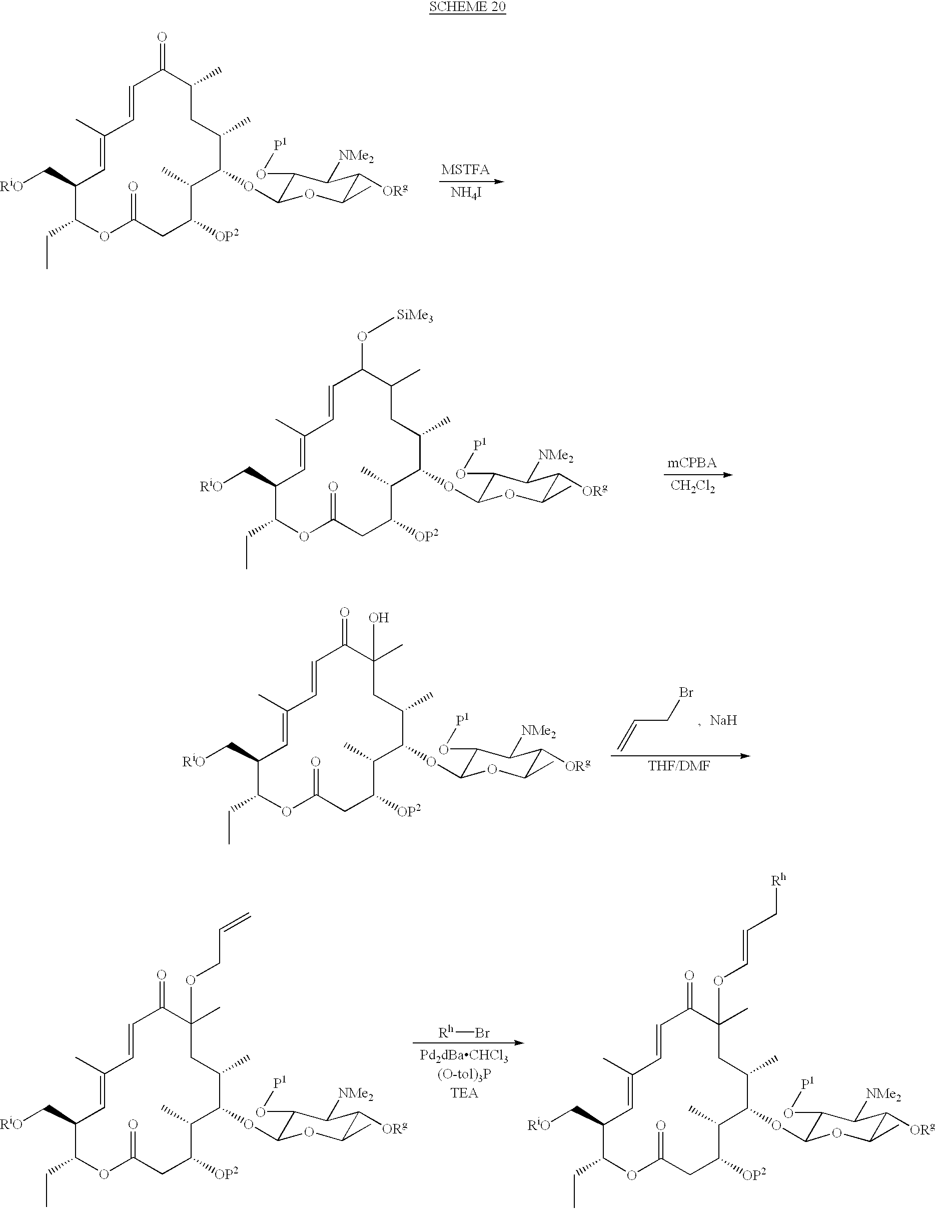

The 8-hydroxyl compound is made as described in Scheme 12. A suitably protected macrolide is treated for example, N-methyl-N-(trimethylsilyl)-trifluoroacetamide to make a silyl enolether. The resulting product is treated with meta-chloroperbenzoic acid in a Rubottom oxidation to yield the corresponding 8-hydroxy compound and alkylated using a base and R′X′ where R′ is an aliphatic, aryl or alkylaryl possessing a terminal double or triple bond and X′ is halide. In preferred embodiments, R′X′ is CH₂═CH—(CH₂)ₙX′ where n is 0-5. In one method and as shown in Scheme 20, R′X′ is allylbromide. In another method, R′X′ is butenylbromide. In yet another method, R′X′ is pentenylbromide. The 8-alkenyl or 8-alkynyl compound is treated with arylhalide such as R^{h}Br under Heck conditions. Depending on the nature of the palladium catalyst and/or phosphine that is used, the double bond in the allyl group can shift in the coupled product as shown in Scheme 20. The resulting product is deprotected as desired. Examples 34-40 describe specific embodiments of this method.

Scheme 21 illustrates another alkylation method and another heck coupling method with reference to a 6-hydroxy-6-vinyl macrolide.

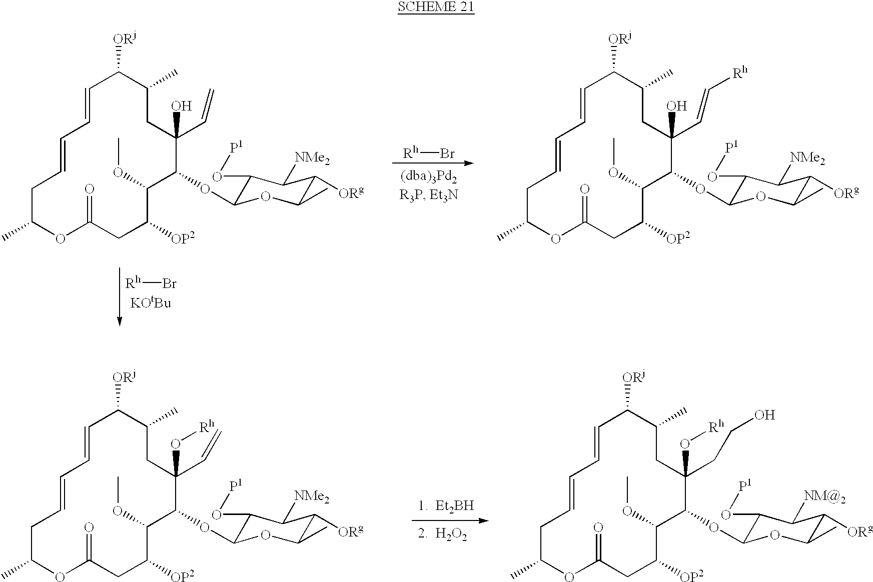

The 6-hydroxy-6-vinyl compound is prepared as described by Scheme 13 where P¹ and P² are each independently a hydroxy protecting group, R^{g} is a hydroxy protecting group or a 3′″,4′″-protected mycarose, and R^{j} is a hydroxy protecting group, forosamine, or 3″,4″-protected mycarose. In one method, a suitably protected 6-hydroxy-6-vinyl compound is treated with an arylhalide under Heck conditions to yield the corresponding 6-hydroxy-6-vinylaryl compound. In another method, a suitably protected 6-hydroxy-6-vinyl compound is alkylated using an alkylating agent such as R^{h}Br where R^{h} is aliphatic, aryl or alkylaryl. Optionally, the vinyl group is converted into a hydroxyethyl which in turn can be further modified. In one embodiment, the vinyl group is hydroborated using diethylborohydride or 9-borabicyclo[3.3.1]nonane (9-BBN) and subsequently oxidized to a hydroxyethyl. Examples 27-33 describe specific embodiments of this method.

In another aspect of the present invention, methods are provided for making sixteen-membered macrolides possessing a side chain Z at C-9 that is ═NOR^{c} where R^{c} is hydrogen, aliphatic, aryl or alkylaryl. A 9-oxo macrolide is converted into an oxime using for example, hydroxyamine. The oxime is optionally treated with R′X′ where R′ is an aliphatic, aryl or alkylaryl possessing a terminal double bond or triple bond and X′ is halide, preferably with CH₂═CH—(CH₂)ₙX′ where n is 0-5. In one method, R′X′ is allylbromide. In another method, R′X′ is butenylbromide. In yet another method, R′X′ is pentenylbromide. Optionally, the alkylated oxime is further modified by treating with arylhalide such as R^{h}Br under Heck conditions.

In another aspect of the present invention, methods are provided for making sixteen-membered macrolides possessing a side chain Z at C-12 that is —WR^{c} wherein R^{c} is hydrogen, aliphatic, aryl or alkylaryl and W is NR^{d} where R^{d} is hydrogen, aliphatic, aryl or alkylaryl. Scheme 22 illustrates one method of making such compounds from a 12,13-epoxy-10-ene-9-one macrolide.

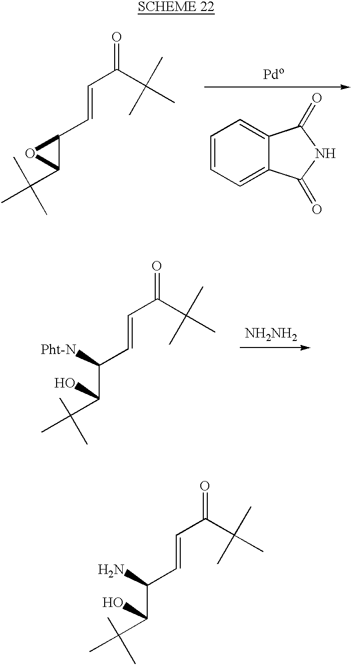

As illustrated in Scheme 22, a 12,13-epoxy-10-ene-9-one macrolide is treated with a palladium metal catalyst (e.g., 1,2-diaminocyclohexane-N,N′-bis(2′-diphenylphosphinobenzoyl) Pd catalyst) and a nucleophile that is capable of acting as a protected amine. In one embodiment, the nucleophile is phthalimide which when treated with for example hydrazine, yields an amino moiety at C-12. In another embodiment, the nucleophile is a carbamate which when removed yields an amino moiety at C-12. In another embodiment, the nucleophile is azide that is reduced using a reducing agent to a C-12 amine once the C-13 hydroxyl is protected. In one method, the C-12 amino group is further modified by acylation using for example an acid chloride. In another method the C-12 amino group is further modified by alkylation with a suitable alkylating agent in the presence of base. In another method, the C-12 amino group is further modified using reductive amination by treating the compound with an aldehyde and sodium cyanoborohydride.

In another aspect of the present invention, methods are provided for making sixteen-membered macrolides possessing a side chain Z at C-13 that is O-aliphatic, O-aryl or O-alkylaryl. A 12-protected-amino-13-hydroxy compound (e.g., 12-carbamyl-13-hydroxyl compound as made for example as described above) is reacted with a suitable alkylating agent in the presence of base. In one embodiment, the alkyl group possesses a terminal double bond that is further modified using olefin metathesis. In another embodiment, the alkyl group possesses a terminal double or triple bond that is further modified with an aryl group under Heck coupling conditions. The phthalimidyl moiety is optionally deprotected using for example hydrazine.

Bicyclic Compounds

In another aspect of the present invention, methods for making bridged bicyclic compounds are provided where one of the cyclic components is a sixteen-membered macrolactone and the other is a cyclic moiety formed by between 5 and 10 atoms. In one embodiment, methods are provided for making bridged bicyclic compounds where the C-9 through C-11 atoms of a sixteen-membered macrolide form the bridge atoms. Scheme 23A illustrates one method of making these compound from a 10-en-9-one macrolide.

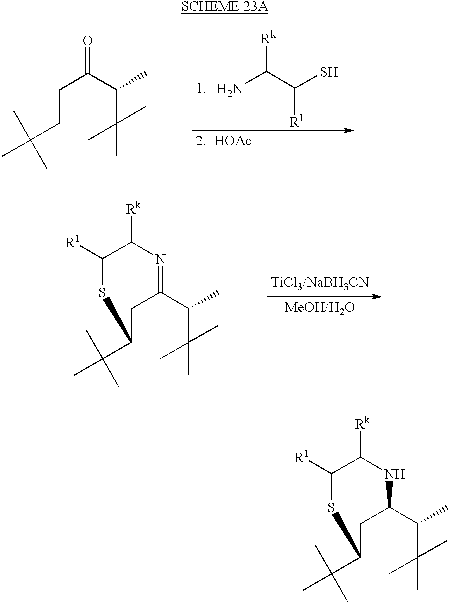

An enone is treated with an aminothiol (where R^{k} and R¹ are each independently hydrogen, aliphatic, aryl, or alkylaryl) in a Michael addition. Treatment with an acid, such as acetic acid, forms the imine that is optionally subsequently reduced with titanium trichloride and sodium cyanoborohydride to yield the indicated product. The C-9 amino group is optionally further modified. Example 42 describes a specific embodiment of this method.

Two illustrative examples of further modifications are shown in Scheme 23B.

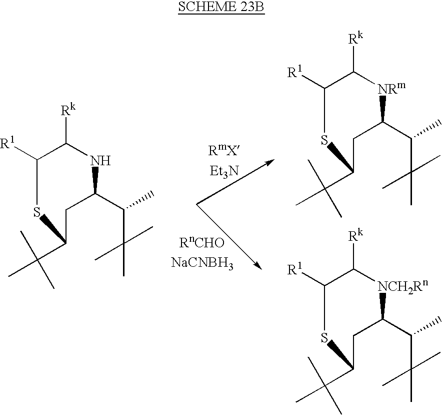

In one method, the C-9 nitrogen is alkylated with an alkylating agent such as R^{m}X′ where R^{m} is aliphatic, aryl or alkylaryl and X′ is a halide in a base such as Et₃N. In another method, the C-9 nitrogen is modified using reductive amination conditions (i.e., aldehyde RⁿCHO and sodium cyanoborohydride where Rⁿ is aliphatic, aryl or alkylaryl).

In another embodiment, methods are provided for making bridged bicyclic compounds where C-11 through C-13 of the sixteen-membered macrolactone for the bridge atoms. Scheme 24A illustrates one method of making these compound from a 12,13-epoxy-10-en-9-one macrolide.

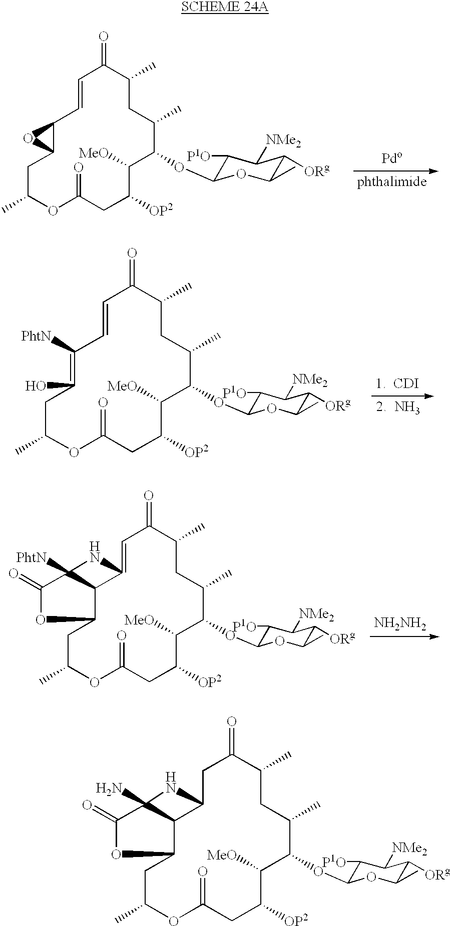

A suitably protected 12, 13, epoxy-10-ene 9-one (where P¹ and P² are each independently a hydroxy protecting group and R^{g} is a hydroxy protecting group, or a 3″,4″-protected mycarose) is treated with a palladium metal catalyst (e.g. 1,2-diaminocyclohexane-N,N′-bis(2′-diphenylphosphinobenzoyl) Pd catalyst) and a nucleophile that is capable of acting as amino protecting group (e.g., phthalimide). The 12-phthalimidyl-13-hydroxy compound is cyclized using for example, carbonyl duiimidazole and ammonia. In other embodiments, an N-substituted cyclic carbamate is formed by using a substituted amine instead of ammonia. See for example, PCT Publications WO 00/62783, WO 00/63224 and WO 00/63225 which are each incorporated herein by reference. Optionally, the phthalimide moiety is deprotected using for example hydrazine and the resulting macrolide is deprotected as desired.

In another embodiment, C-12 amino group of the 12-amino-11,13-carbamate compound described in Scheme 24A is further modified. Two illustrative modifications are described in Scheme 24B.

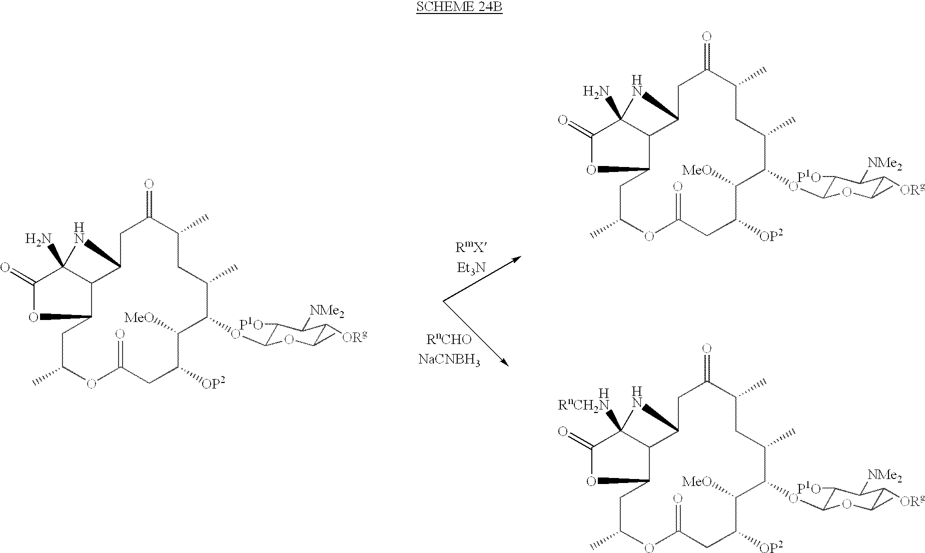

In one method, the 12-amino-11,13-carbamate is alkylated with an alkylating agent such as an alkyl halide where R^{m} is aliphatic, aryl, or alkylaryl and X′ is a halide in a base. In another embodiment, the 12-amino-11,13-carbamate is subject to reductive amination conditions using aldehyde RⁿCHO and sodium cyanoborohydride where Rⁿ is alkyl, aryl or alkylaryl. In yet another embodiment, the 12-amino-11,13-carbamate is acylated using for example, an acid chloride.

In another aspect of the present invention, methods for making fused bicyclic compounds are provided where one of the cyclic components is a sixteen-membered macrolactone and the other is a cyclic moiety formed by between 3 and 10 atoms. In one embodiment, radical-mediated cyclization methods are provided to convert an acyl halide-containing macrolide into a fused bicyclic compound. Scheme 25 describes one method with reference to a 14-hydroxymethyl macrolide for the purposes of illustration.

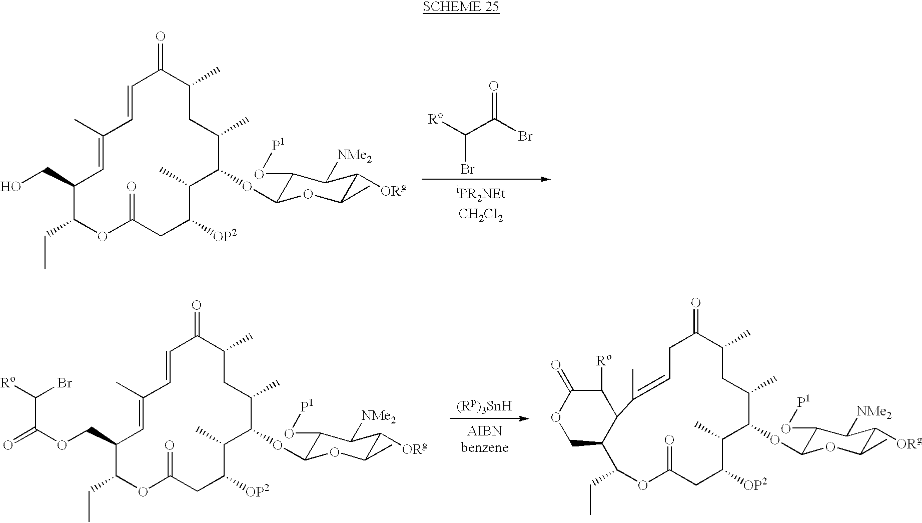

A 14-hydroxymethyl macrolide (where P¹ and P² are each independently a hydroxy protecting group and R^{g} is a hydroxy protecting group, or a 3′,4′-protected mycarose) is treated with an acyl halide in a base such as diisopropylethylamine or pyrimidine. Suitable examples of acylhalides include α-bromoacylbromide where R^{o} is hydrogen, aliphatic, aryl or alkylaryl that can be obtained by reacting carboxylic acids with bromine and phosphorus tribromide. The acylbromide moiety is then cyclized using a radical generated by trialkyl tin hydride and azobis(cyclohexane carbonitrile) or other suitable radical generating species. In one embodiment, R^{o} includes a terminal double bond that is further modified by metathesis. In another embodiment, R⁰ includes a terminal double or triple bond that is further modified by Heck coupling. The resulting product is deprotected as desired. Examples 43-46 describe specific embodiments of this method.

In another embodiment, radical-mediated cyclization methods are provided to convert a propargyl-containing macrolide into a fused bicyclic compound. Scheme 26 describes one method with reference to a 14-hydroxymethyl macrolide for the purposes of illustration.

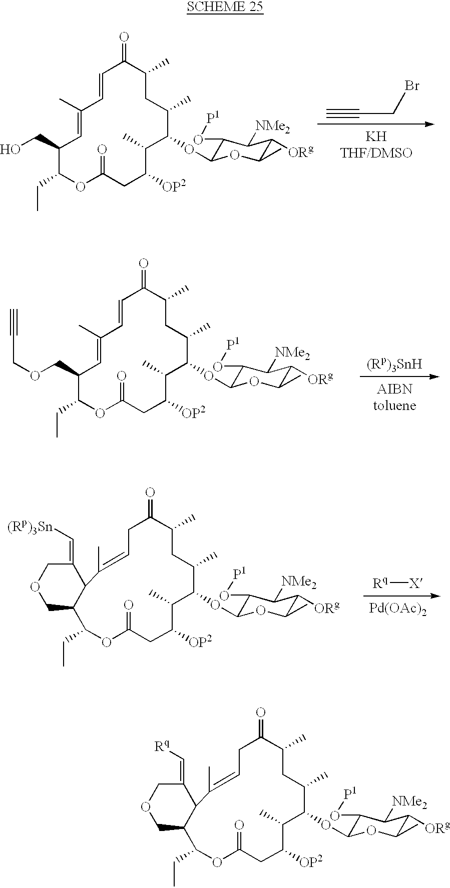

A 14-hydroxymethyl macrolide (where P¹ and P² are each independently a hydroxy protecting group and R^{g} is a hydroxy protecting group, or a 3″,4″-protected mycarose) is treated with a propargyl halide such as propargyl bromide. The —O-propargyl moiety is then cyclized using a trialkyl tin hydride and azobis(cyclohexane carbonitrile), and reacted with an alkyl halide R^{q}X′ where R^{q} is aliphatic, aryl, or alkylaryl in a Stille coupling reaction. The resulting product is deprotected as desired. Examples 47-48 describe specific embodiments of this method.

In another embodiment, zinc-mediated cyclization methods are provided to convert an a-halo-acyl-containing macrolide into a fused bicyclic compound. The acyl halide is formed in a similar manner to that described in Scheme 25. The 14-hydroxymethyl macrolide is treated with an acyl halide in a base such as diisopropylethylamine or pyrimidine. Suitable examples of acylhalides include α-bromoacylbromide where R^{o} is hydrogen, aliphatic, aryl or alkylaryl that can be obtained by reacting carboxylic acids with bromine and phosphorus tribromide. The acylbromide moiety is then cyclized with zinc. The resulting product is deprotected as desired.

In another embodiment, zinc-mediated cyclization methods are provided to convert an α-halo-amide into a fused bicyclic compound. Scheme 27 describes one method with reference to a 14-aminomethyl macrolide for the purposes of illustration.

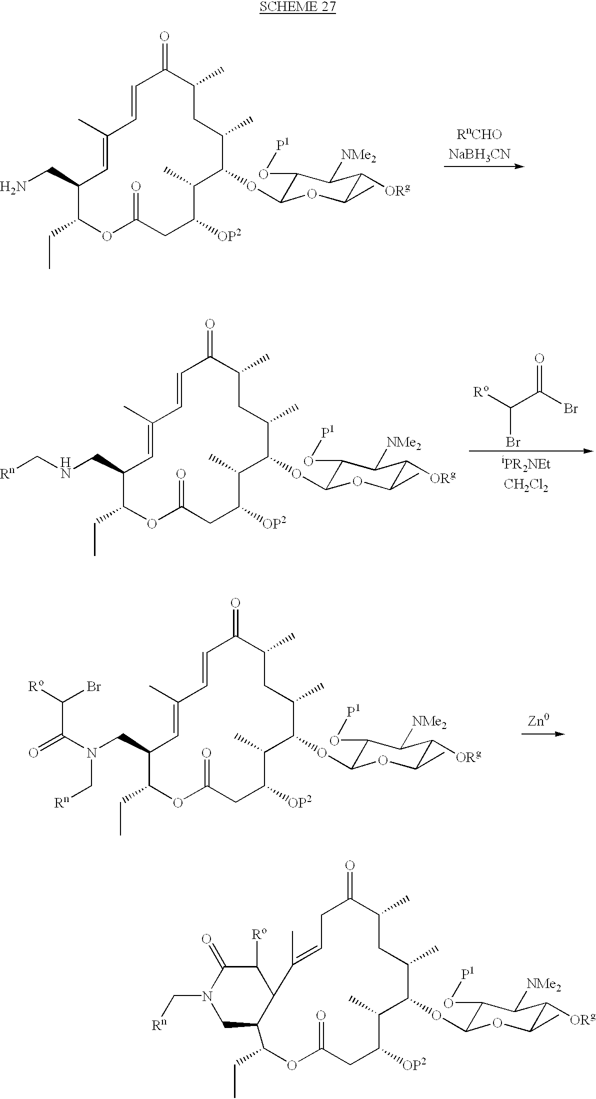

The aminomethyl macrolide (where P¹ and P² are each independently a hydroxy protecting group and R^{g} is a hydroxy protecting group, or a 3″,4″-protected mycarose) is obtained using similar methods as that described by Scheme 17. A 14-hydroxymethyl macrolide is converted into an iodide that is displaced with ammonia to yield the corresponding 14-aminomethyl macrolide. As shown in Scheme 27, the amino nitrogen is alkylated in a reductive amination reaction with aldehyde RⁿCHO and sodium cyanoborohydride. The resulting product is acylated using an a-bromo-acylbromide and then cyclized using zinc, and deprotected as desired.

In another embodiment, base-mediated cyclization methods are provided to convert a urea into a fused bicyclic compound. Scheme 28 describes one method with reference to a 14-aminomethyl macrolide for the purposes of illustration.

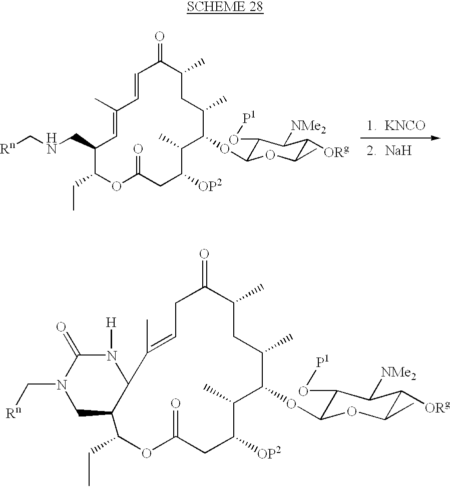

The alkylamino macrolide (where P¹ and P² are each independently a hydroxy protecting group and R^{g} is a hydroxy protecting group, or a 3″,4″-protected mycarose) is obtained using similar methods as that described by Scheme 27. As shown in Scheme 28, the alkylamino moiety is converted into a urea by reaction with an isocynate such as KNCO or Me₃SiNCO and then cyclized using a base such as sodium hydride. The resulting product is deprotected as desired.

In another embodiment, base-mediated cyclization methods are provided to convert a carbamate-containing macrolide into a fused bicyclic compound. Scheme 29 describes one method with reference to a 14-hydroxymethyl macrolide for the purposes of illustration.

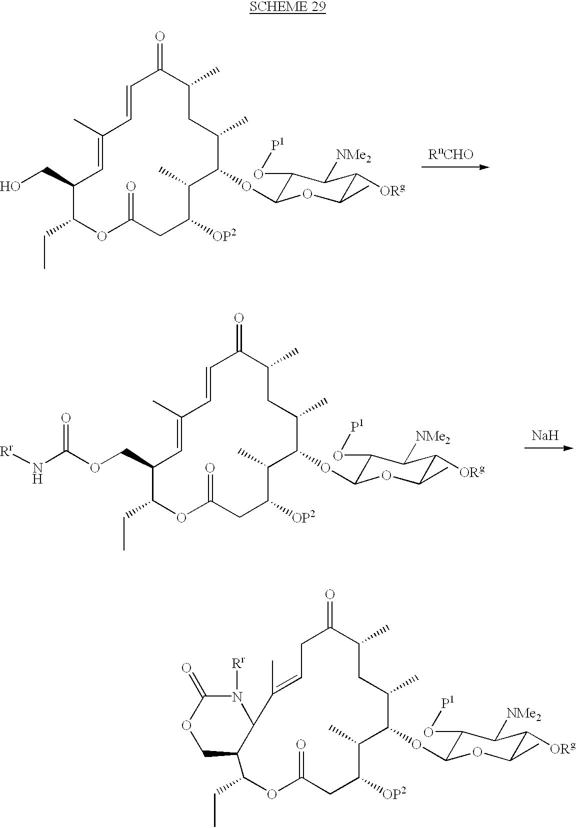

A suitably protected 14-hydroxymethyl macrolide (where P¹ and P² are each independently a hydroxy protecting group and R^{g} is a hydroxy protecting group, or a 3″,4″-protected mycarose) is treated with isocyanate R^{r} NCO (where R^{r} is aliphatic, aryl, or alkylaryl) to yield the corresponding carbamate. The carbamate is then cyclized using a base such as sodium hydride and deprotected as desired.

In another embodiment, sodium hydride-mediated cyclization methods are provided to convert a 9-oxo-10,12-dienyl-12-hydroxymethyl macrolide to a 9-oxo-12-ene-11,12-cyclic carbamate. In one method, 9-oxo-10,12-dienyl-12-hydroxymethyl macrolide is cyclized using carbonyldiimidazole/sodium hydride and ammonium hydroxide to yield the corresponding 9-oxo-12-ene-11,12-cyclic carbamate

In another embodiment, methods are provided for converting a diol-containing macrolide into a fused bicyclic compound. Scheme 30A describes one method with reference to a 9-oxo-10-ene macrolide.

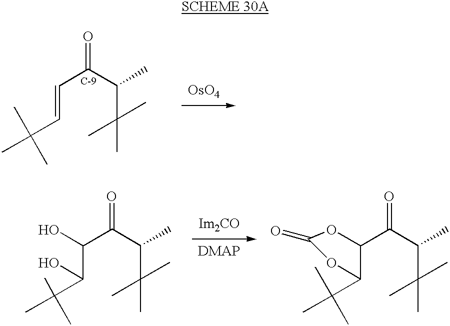

As shown in Scheme 30A, a suitably protected enone is dihydroxylated using for example, osmium tetraoxide. The diol is then converted into a cyclic carbonate using for example phosgene or carbonyldiimidizole. Scheme 30B further describes this method as it applies to OMT (O-mycaminosyltylactone) as the starting material for the purposes of illustration.

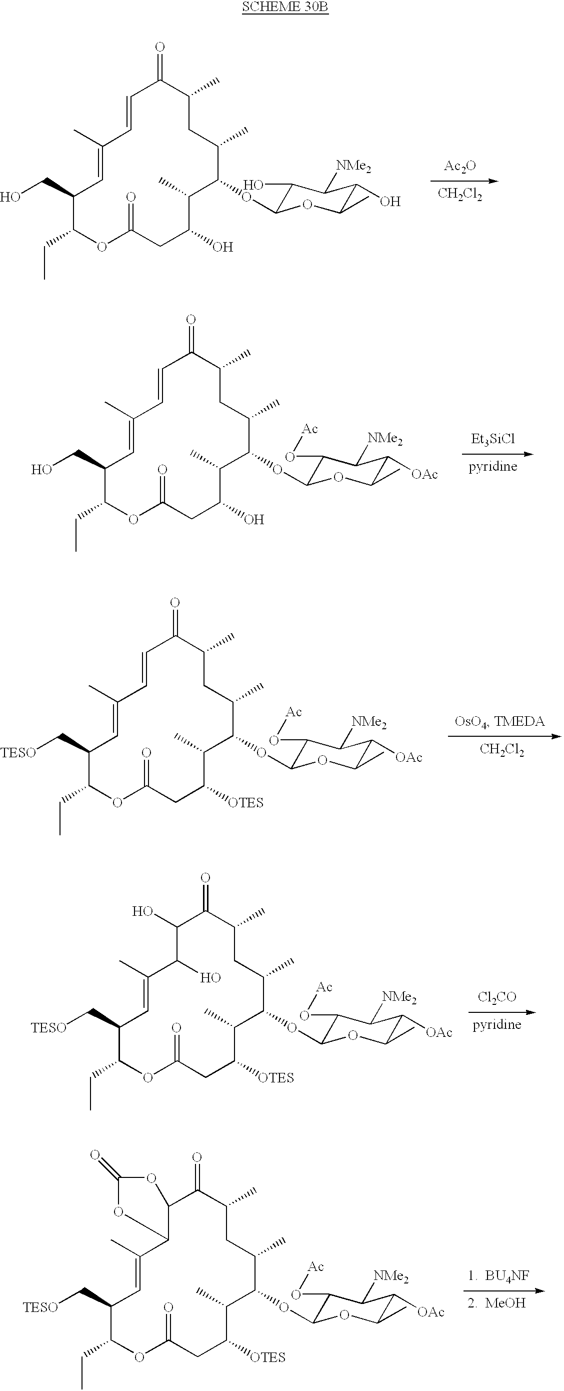

OMT is acetylated to protect the 2′- and 4′ hydroxyls of mycaminose, and silated to protect the C-14 hydroxymethyl. The hydroxy protected OMT is treated with tetramethylethylenediamine and osmium tetraoxide to form the 10,11-diol and cyclized with phosgene. Deprotection of the silyl group and the acetyl groups yields the cyclic carbonate. Examples 49-52 describe specific embodiments of this method.

In another embodiment, methods are provided for converting a diol-containing macrolide into a cyclic acetal. Scheme 31 illustrates one method with reference to a 9-oxo-10,11-dihydroxy macrolide.

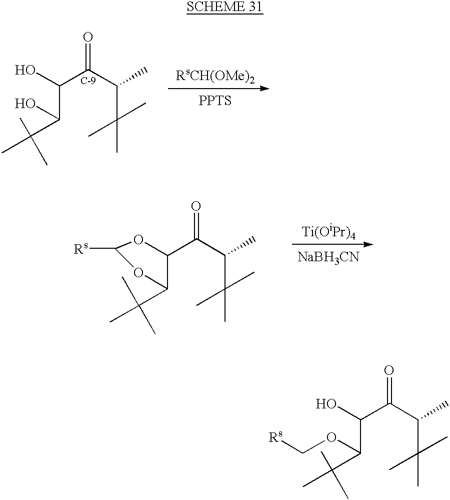

The diol is made as described in Scheme 30A. In one method, a suitably protected diol-containing macrolide is treated with dimethyl acetal R^{s}CH(OMe)₂ (where R^{s} is hydrogen, aliphatic, aryl or alkylaryl) to yield a cyclic acetal. In another method, the cyclic acetal is reduced using for example titanium(IV)isopropoxide and NaBH₃CN to yield a 10-hydroxy-11-OCH₂R^{s}-containing macrolide.

In another embodiment, methods are provided for converting a diol-containing macrolide into a cyclic carbamate. Scheme 32 illustrates one method with reference to a 9-oxo-10,11-dihydroxy macrolide.

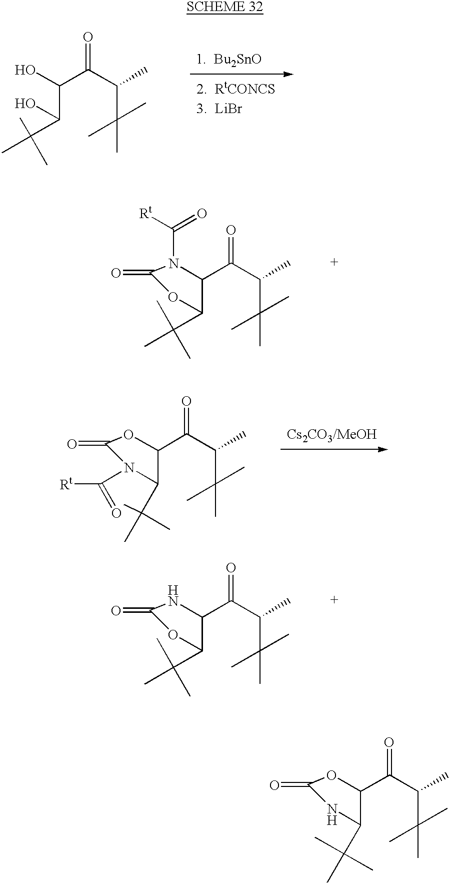

The diol is made as described in Scheme 30A. In one method, a suitably protected diol-containing macrolide is treated with dibutyl tin oxide, acyl thiocyanate R^{t}CONCS (where R^{t} is hydrogen, aliphatic, aryl or alkylaryl), and lithium bromide. In another method, the acyl-carbamate is deacylated using for example cesium carbonate and methanol. Examples 53-55 describe specific embodiments of this method.

In another embodiment, methods are provided for converting a 7-hydroxy-6-enal macrolide into a 9-oxo-6,7-cyclic carbamate. Scheme 33A illustrates one method.

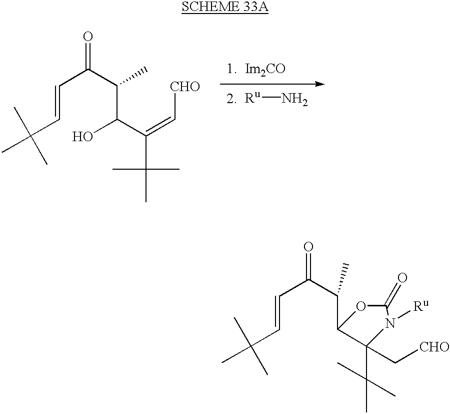

The 7-hydroxy-6-enal is made as described by Scheme 11. A suitably protected 7-hydroxy-6-enal macrolide is treated with carbonyldimidizole and amine R^{u}NH₂. The resulting product is deprotected as desired.

In another embodiment methods are provided for converting a 7-hydroxy-6-enal macrolide into a 9-hydroxy-6,7-cyclic carbamate. Scheme 33B illustrates one method for this conversion.

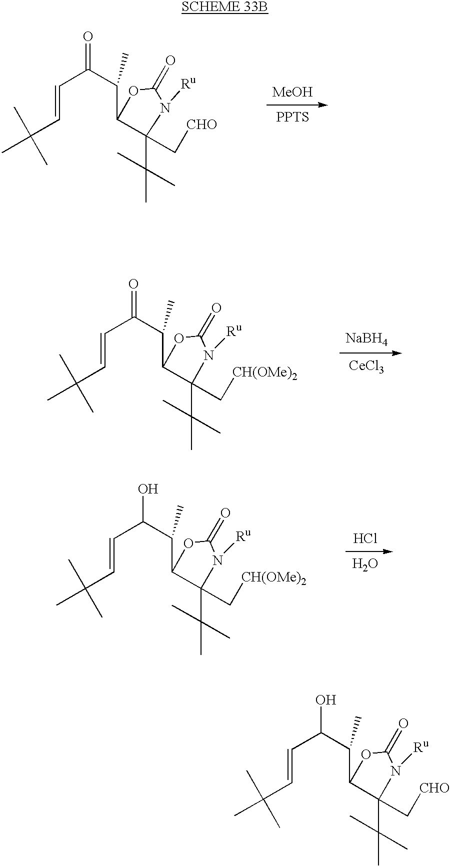

The 9-oxo-6,7-cyclic carbamate macrolide is made as described by Scheme 32A. The aldehyde at C-6 is protected as an acetal and the oxo group at C-9 is reduced using a reducing agent such as sodium borohydride. Removal of the acetal yields the corresponding 9-hydroxy-6,7-cyclic carbamate. Scheme 33C illustrates another method for converting a 7-hydroxy-6-enal macrolide into a 9-hydroxy-6,7,-cyclic carbamate.

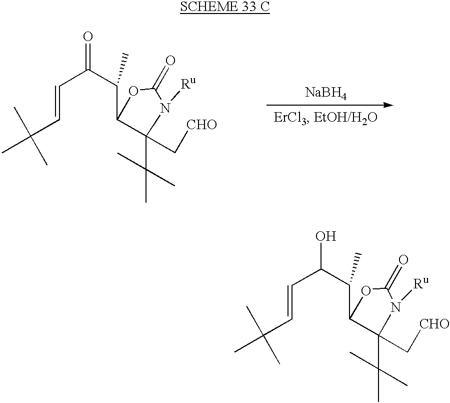

The 9-oxo-6,7-cyclic carbamate macrolide is reduced to a 9-hydroxy-6,7-cyclic carbamate macrolide using sodium borohydride and erbium trichloride in the presence of ethanol and water.

In another embodiment, methods are provided for converting a 9-amino-10-ene macrolide into 9,10-cyclic urea macrolide. Scheme 34 illustrates one method for this conversion.

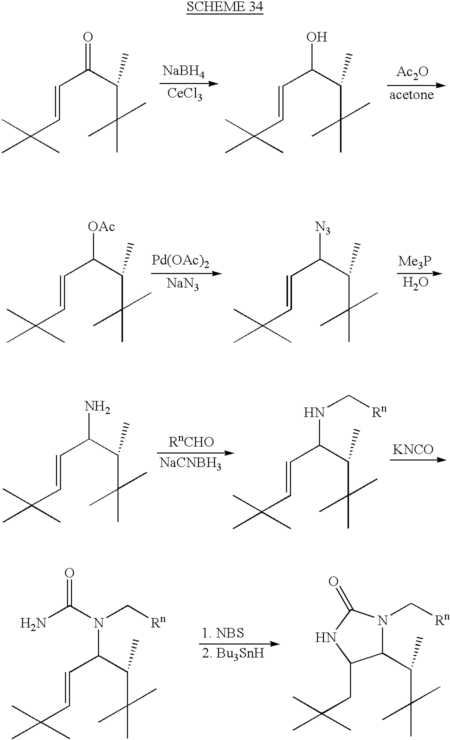

In one method, a 10-ene-9-one is converted into a 10-ene-9-hydroxy macrolide using a reducing agent such as sodium borohydride. In another method, the starting macrolide is a 10-ene-9-hydroxy macrolide. As shown in Scheme 34, the hydroxy group at C-9 is converted into an acetate moiety and is displaced with an azide using palladium mediated nucleophillic displacement. The C-9 azide is then reduced to an amine and converted into a urea using for example, potassium isocyanate. The C-9 urea is then cyclized by treating with N-bromosuccimide and tributyltin hydride.

In another embodiment, methods are provided for converting a 12,13-epoxy-10-ene-9-one macrolide into a 13-hydroxy-11,12-cyclic urea macrolide. One method for this conversion is shown in Scheme 35A.

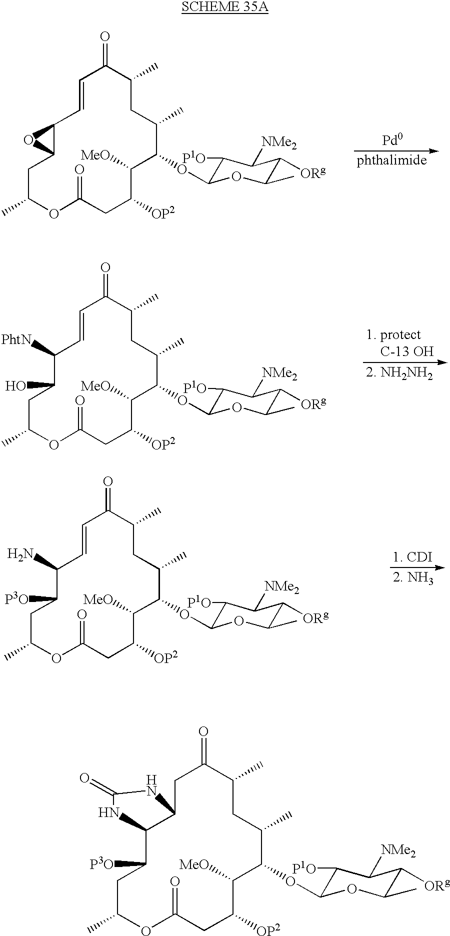

A 12,13-epoxy-i10-ene-9-one macrolide is treated with a palladium metal catalyst (e.g., 1,2-diaminocyclohexane-N,N′-bis(2′diphenylphosphinobenzoyl) Pd catalyst) and phthalimide. The 12-phthalimidyl-13-hydroxy group is treated with a hydroxy protecting group and then treated with hydrazine to remove the phthalimidyl moiety. The resulting product is cyclized using carbonyl diimidazole and ammonia to yield the 11, 12 cyclic urea macrolide. In one embodiment, the C-13 hydroxyl is selectively deprotected and further modify such as ether formation or allylation followed by Heck coupling reactions.

In another embodiment, methods are provided for converting a 12,13-epoxy-10-ene-9-one macrolide into a 13-hydroxy-11,12-cyclic carbamate macrolide. One method for this conversion is shown in Scheme 35B

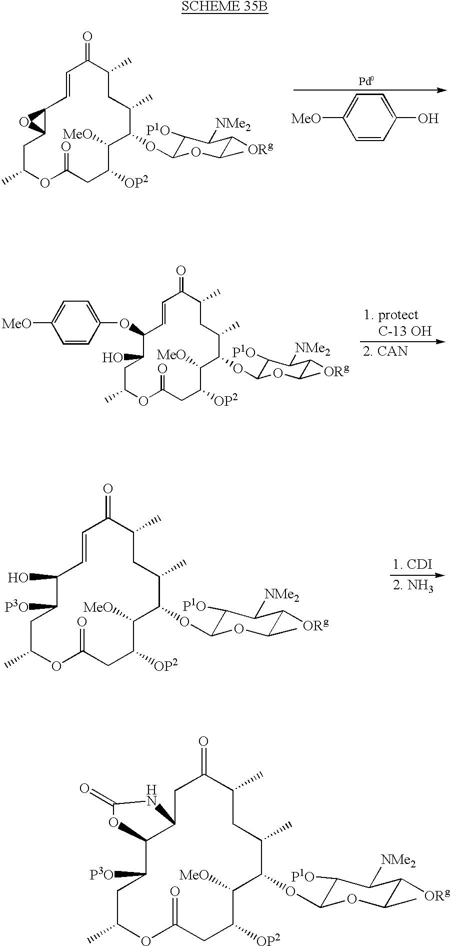

A 12,13-epoxy-10-ene-9-one macrolide is treated with a palladium metal catalyst (e.g., 1,2-diaminocyclohexane-N,N′-bis(2′diphenylphosphinobenzoyl) Pd catalyst) and para-methoxy-phenol. The 12-para-methoxy-phenolyl-13-hydroxy group is treated with a hydroxy protecting group and then treated with cerium (IV) ammonium nitrate to remove the phenolic moiety. The resulting product is cyclized using carbonyl diimidazole and ammonia to yield the 11, 12 cyclic carbamate macrolide. In one embodiment, a substituted amine is used instead of ammonia in the cyclization reaction to yield an N-substituted cyclic carbamate. In another embodiment, the C-13 hydroxyl is selectively deprotected and further modify such as ether formation or allylation followed by Heck coupling reactions.

In another embodiment, methods are provided for making a 11, 12 cyclic carbamate starting from a C-13 allylic alcohol. Scheme 36A describes one method with reference to a 19-deformyl macrolide for the purposes of illustration (where P¹ and P² are each independently a hydroxy protecting group; R^{g} is a hydroxy protecting group, or a 3″,4″-protected mycarose, and Rⁱ is a hydroxy protecting group or a 4′″ protected mycinose).

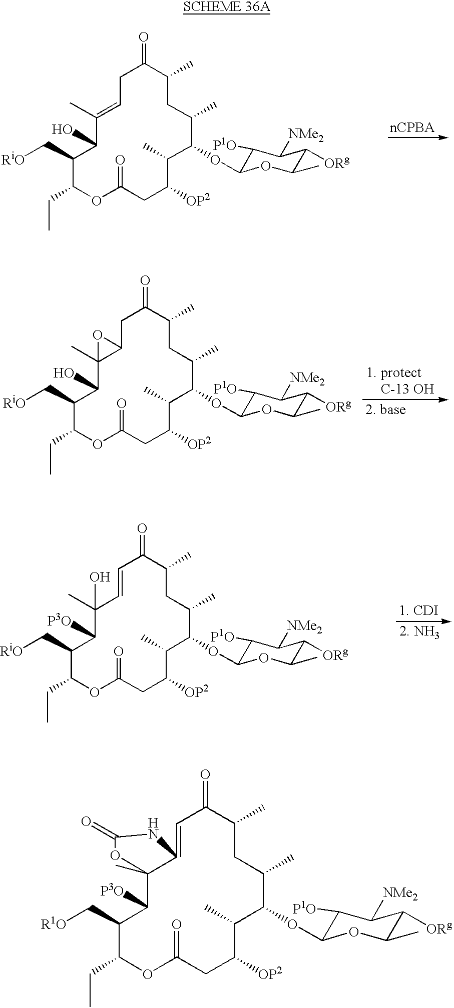

The C-13 allylic alcohol-containing macrolide is made as described by Scheme 14. The 12,13-double bond is epoxidized using an epoxidating agent such as mCPBA. The C-13 hydroxyl is protected and the resulting product is treated with a base to yield the 12-hydroxy-10,11-enone. In one embodiment, the enone is treated with carbonyl diimidazole and ammonia to yield the 11,12-cyclic carbamate. In another embodiment, the enone is treated with carbonyl diimidazole and a substituted amine to yield an N-substituted cyclic carbamate-containing macrolide. The resulting product is deprotected as desired.

In another embodiment, methods are provided for making a 12, 13 cyclic carbonate starting from a C-13 allylic alcohol. Scheme 36B describes one method with reference to a 19-deformyl macrolide for the purposes of illustration (where P¹ and P² are each independently a hydroxy protecting group; R^{g} is a hydroxy protecting group, or a 3″,4″-protected mycarose, and Rⁱ is a hydroxy protecting group or a 4′″ protected mycinose).

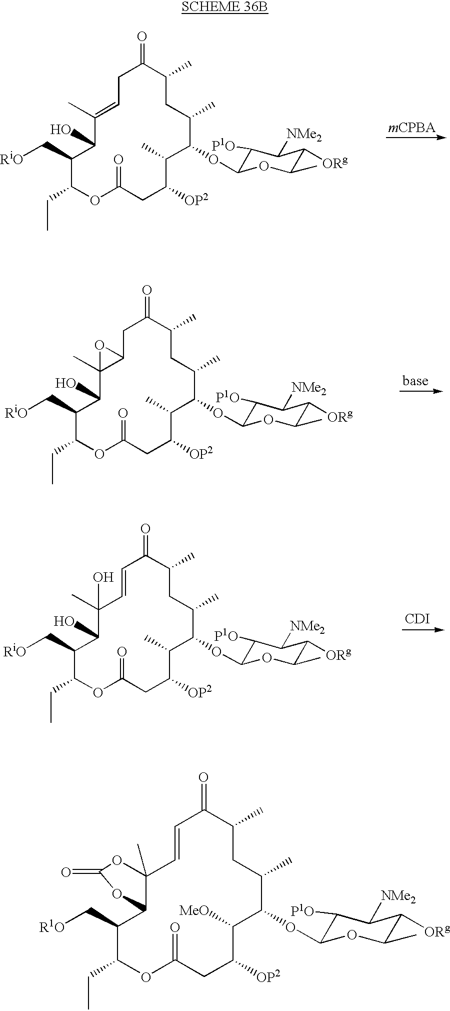

The C-13 allylic alcohol-containing macrolide is made as described by Scheme 14. The 12,13-double bond is epoxidized using an epoxidating agent such as mCPBA and treated with a base to yield the 12-hydroxy-10,11-enone. The enone is treated with carbonyl diimidazole to yield the 12,13-cyclic carbonate. The resulting product is deprotected as desired.

Purification and Characterization

Compounds are purified by preparative HPLC, and characterized using high-resolution LC/MS and multi-nuclear NMR. A combination of COSY, [¹H,¹³C]-HSQC, and [¹H,¹³C]-HMBC experiments provide the requisite atomic connectivity.

Ribosome Binding Studies

Binding to domain II is measured using ribosomes from S. pneumoniae and from resistant strains. See Weisblum, B. (1995) Antimicrob. Agents Chemother. 39, 577-585; NCCLS Report MO7-A5 (2000) Vol. 20; Goldman, R. C., and Kadama, S. K. (1989) Antimicrob. Agents Chemother. 33, 1058-1066 which are each incorporated herein. In one embodiment, the K_{d}s using ribosomes from sensitive and resistant strains are taken as a surrogate for (or a measure of ) domain II binding. For example, the K_{d} of erythromycin A about 14 nM using wildtype ribosomes is and about 190,000 nM using methylated ribosome (e.g., A2058G mutant). In contrast, the K_{d} of telithromycin (HMR 3647) is about 1.3 nM using wildtype ribosomes is and about 58 nM using methylated ribosome (e.g. A2058G mutant). Thus in one method, a K_{d} of about less than or equal to about 50 nM using wildtype ribosomes and a K_{d} of less than or equal to about 10000 nM is a surrogate marker for domain II binding. In another method, a K_{d} of about less than or equal to about 50 nM using wildtype ribosomes and a K_{d} of less than or equal to about 1000 nM is a surrogate marker for domain II binding. In another method, a K_{d} of about less than or equal to about 50 nM using wildtype ribosomes and a K_{d} of less than or equal to about 500 nM is a surrogate marker for domain II binding. In another method, a K_{d} of about less than or equal to about 50 nM using wildtype ribosomes and a K_{d} of less than or equal to about 250 nM is a surrogate marker for domain II binding. In another method, a K_{d} of about less than or equal to about 50 nM using wildtype ribosomes and a K_{d} of less than or equal to about 100 nM is a surrogate marker for domain II binding. In another method, a K_{d} of about less than or equal to about 25 nM using wildtype ribosomes and a K_{d} of less than or equal to about 75 nM is a surrogate marker for domain II binding. In another method, a K_{d} of about less than or equal to about 10 nM using wildtype ribosomes and a K_{d} of less than or equal to about 60 nM is a surrogate marker for domain II binding.

In another embodiment, displacement of erythromycin and/or telithromycin using ribosomes for sensitive and resistant strains is taken as a surrogate marker for domain II binding. These experiments are performed using ¹⁴C-labeled erythromycin (obtainable commercially) and labeled HMR-3647. ³H-HMR-3647 is prepared according to Zhong, P., Cao, Z., Hammond, R., Chen, Y., Beyer, J., Shortridge, V. D., Phan, L. Y., Pratt, S., Capobianco, J., Reich, K. A., Flamm, R. K., Or, Y. -S., and Katz, L. (1999) Microb. Drug Resist. 5, 183-188 which is incorporated herein by reference.

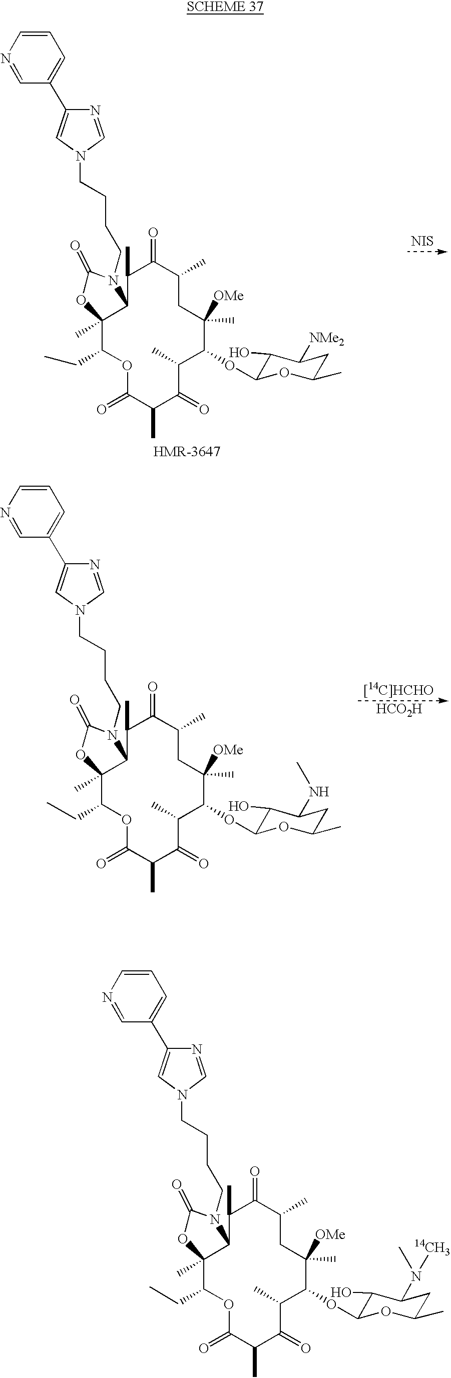

Scheme 37 illustrates one method for making ¹⁴C-HMR-3647. Briefly, HMR-3647 is demethylated on the 3′-nitrogen of desosamine and reductively alkylated to add a [¹⁴C]-methyl group. N-demethylation is carried out using N-iodosuccinimide instead of iodine to avoid iodination at the C-2 position of the macrolide ketoester. The demethylated ketolide is subjected to reductive amination with [¹⁴C]-formaldehyde to introduce the label using an Eschweiler-Clarke type procedure (HCHO+HCO₂H) or sodium cyanoborohydride reduction.

In addition to ribosomal binding, peptidyl transferase activity is measured. An illustrative assay which may be used to measure peptidyl transferase inhibition is described by Cannon, European J. Biochem. 7: 137-145 (1968) which is incorporated herein by reference.

MIC Analysis

The minimal inhibitory concentration (“MIC”) for test compounds is determined against a panel of organisms including macrolide-susceptible staphylococci, streptococci (penicillin-susceptible and resistant strains), Haemophilus influenzae, Moraxella catarrhalis and vancomycin-resistant Entercoccus faecalis. The compounds are also tested against erythromycin-resistant strains including constitutive and inducible MLS and efflux pump-containing strains of Staphylococcus aureus and Streptoccus pneumoniae. Compounds can be further screened against a broader panel of organisms including gram negative rods, atypical pathogens, and a larger panel of macrolide-resistant recent clinical isolates. The MICs are determined by the microdilution method according to procedures established by the National Committee on Clinical Laboratory Standards (“NCCLS”), with macrolide antibiotics erythromycin, clarithromycin, HMR-3647, ABT 773 and tylosin and with other major antibiotic classes represented by ampicillin, vancomycin, tetracycline, gentamicin and a fluoroquinolone as reference standards.

Formulation and Methods of Use

Methods for treating a patient in need of an anti-infective agent generally comprise administering a therapeutically effective amount of an inventive compound to a subject in need thereof.

A composition of the present invention generally comprises an inventive compound and a pharmaceutically acceptable carrier. The inventive compound may be free form or where appropriate as pharmaceutically acceptable derivatives such as prodrugs, and salts and esters of the inventive compound.

The composition may be in any suitable form such as solid, semisolid, or liquid form. See Pharmaceutical Dosage Forms and Drug Delivery Systems, 5^{th} edition, Lippicott Williams & Wilkins (1991) which is incorporated herein by reference. In general, the pharmaceutical preparation will contain one or more of the compounds of the invention as an active ingredient in admixture with an organic or inorganic carrier or excipient suitable for external, enteral, or parenteral application. The active ingredient may be compounded, for example, with the usual non-toxic, pharmaceutically acceptable carriers for tablets, pellets, capsules, suppositories, pessaries, solutions, emulsions, suspensions, and any other form suitable for use. The carriers that can be used include water, glucose, lactose, gum acacia, gelatin, mannitol, starch paste, magnesium trisilicate, talc, corn starch, keratin, colloidal silica, potato starch, urea, and other carriers suitable for use in manufacturing preparations, in solid, semi-solid, or liquified form. In addition, auxiliary stabilizing, thickening, and coloring agents and perfumes may be used.

Where applicable, the inventive compounds may be formulated as microcapsules and nanoparticles. General protocols are described for example, by Microcapsules and Nanoparticles in Medicine and Pharmacy by Max Donbrow, ed., CRC Press (1992) and by U.S. Pat. Nos. 5,510,118; 5,534,270; and 5,662,883 which are all incorporated herein by reference. By increasing the ratio of surface area to volume, these formulations allow for the oral delivery of compounds that would not otherwise be amenable to oral delivery.

The inventive compounds may also be formulated using other methods that have been previously used for low solubility drugs. For example, the compounds may form emulsions with vitamin E or a PEGylated derivative thereof as described by WO 98/30205 and 00/71163which are incorporated herein by reference. Typically, the inventive compound is dissolved in an aqueous solution containing ethanol (preferably less than 1% w/v). Vitamin E or a PEGylated-vitamin E is added. The ethanol is then removed to form a pre-emulsion that can be formulated for intravenous or oral routes of administration.

Yet another method involves formulating the inventive compounds using polymers such as polymers such as biopolymers or biocompatible (synthetic or naturally occurring) polymers. Biocompatible polymers can be categorized as biodegradable and non-biodegradable. Biodegradable polymers degrade in vivo as a function of chemical composition, method of manufacture, and implant structure. Illustrative examples of synthetic polymers include polyanhydrides, polyhydroxyacids such as polylactic acid, polyglycolic acids and copolymers thereof, polyesters polyamides polyorthoesters and some polyphosphazenes. Illustrative examples of naturally occurring polymers include proteins and polysaccharides such as collagen, hyaluronic acid, albumin, and gelatin.

Another method involves conjugating the compounds of the present invention to a polymer that enhances aqueous solubility. Examples of suitable polymers include polyethylene glycol, poly-(d-glutamic acid), poly-(1-glutamic acid), poly-(1-glutamic acid), poly-(d-aspartic acid), poly-(1-aspartic acid), poly-(1-aspartic acid) and copolymers thereof. Polyglutamic acids having molecular weights between about 5,000 to about 100,000 are preferred, with molecular weights between about 20,000 and 80,000 being more preferred and with molecular weights between about 30,000 and 60,000 being most preferred.

Dosage levels of the compounds of the present invention are of the order from about 0.01 mg to about 100 mg per kilogram of body weight per day, preferably from about 0.1 mg to about 50 mg per kilogram of body weight per day. More preferably, the dosage levels are from about 0.7 mg to about 3.5 mg per patient per day, assuming a 70 kg patient. In addition, the compounds of the present invention may be administered on an intermittent basis, i.e., at semi-weekly, weekly, semi-monthly, or monthly intervals.

The amount of active ingredient that may be combined with the carrier materials to produce a single dosage form will vary depending upon the host treated and the particular mode of administration. For example, a formulation intended for oral administration to humans may contain from 0.5 mg to 5 gm of active agent compounded with an appropriate and convenient amount of carrier material, which may vary from about 5 percent to about 95 percent of the total composition. Dosage unit forms will generally contain from about 0.5 mg to about 500 mg of active ingredient. For external administration, the compounds of the invention may be formulated within the range of, for example, 0.00001% to 60% by weight, preferably from 0.001% to 10% by weight, and most preferably from about 0.005% to 0.8% by weight.

It will be understood, however, that the specific dose level for any particular patient will depend on a variety of factors. These factors include the activity of the specific compound employed; the age, body weight, general health, sex, and diet of the subject; the time and route of administration and the rate of excretion of the drug; whether a drug combination is employed in the treatment; and the severity of the particular disease or condition for which therapy is sought.

In summary, novel sixteen-membered macrolide compounds have been provided that are useful as anti-infective agents or intermediates thereto. Although specific examples have been used to illustrate the present invention, the particular embodiments described herein are for the purposes of illustration only and are not intended to limit the scope of the present invention

### EXAMPLE 1

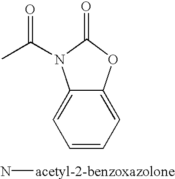

Acetic anhydride(15mL)was added to as stirred suspension of 2-benzoxazolone (13.5 g) and potassium carbonate (1.4 g) in acetone (50 mL). After 12 hours, the mixture was poured into 1000 mL of water, and the precipitated product was collected by vacuum filtration. The product was dried under vacuum, and crystallized from CH₂Cl₂/Hexanes. ¹³C-NMR (CDCl₃): δ 169.4, 151.5, 142.1, 127.6, 125.3, 124.8, 115.9, 109.8,24.9.

### EXAMPLE 2

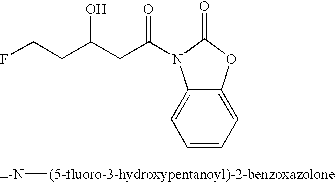

Step 1 o-Iodoxybenzoic acid (7.90 g, 28.2 mmol) was added to DMSO (19 mL) and stirred for 20 minutes until dissolved. 3-Fluoropropanol (2.0 g, 25.6 mmol) was added and the resulting mixture was stirred for 3 hours. The 3-fluoropropanal was distilled directly from the reaction vessel and condensed at −78° C. The distillate was dissolved in methylene chloride (10 mL) and used directly in the subsequent reaction.

Step 2. Titanium tetrachloride (2.81 mL, 25.6 mmol) is added to a solution of N-acetyl-2-benzoxazolone (4.5 g, 25.6 mmol) in methylene chloride (50 mL) at −15° C. (methanol/ice bath) and stirred 5 minutes. Diisopropylethylamine (4.47 mL, 25.6 mmol) is added and the reaction mixture is stirred 15 minutes. The methylene chloride solution of the distillate from the prior reaction is then added and the reaction mixture is stirred and maintained at −15° C. for 1 hour. The excess reagents are quenched by addition of 2N HCl (40 mL). The reaction mixture is extracted with ether (3×100 mL) and the combined ether layers are washed with 2 N HCl (2×50 mL), a saturated sodium bicarbonate solution (50 mL), dried with magnesium sulfate, filtered and evaporated. Silica chromatography (hexanes/ethyl acetate) gives the product.

### EXAMPLE 3

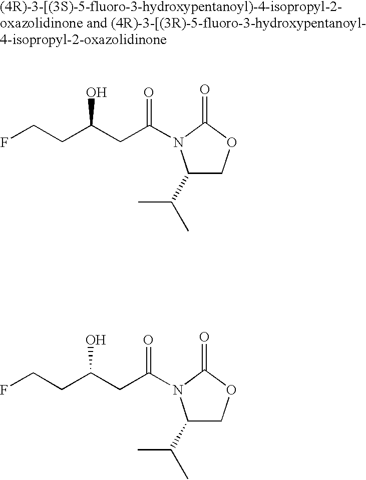

Step 1 o-Iodoxybenzoic acid (7.90 g, 28.2 mmol) was added to DMSO (19 mL) and stirred for 20 minutes until dissolved. 3-Fluoropropanol (2.0 g, 25.6 mmol) was added and the resulting mixture was stirred for 3 hours. The 3-fluoropropanal was distilled directly from the reaction vessel and condensed at −78° C. The distillate was dissolved in methylene chloride (10 mL) and used directly in the subsequent reaction.

Step 2. Titanium tetrachloride (2.81 mL, 25.6 mmol) is added to a solution of (4R)-3-acetyl-4-isopropyl-2-oxazolidinone (4.4 g, 25.6 mmol) in methylene chloride (50 mL) at −78° C. and stirred 10 minutes. Diisopropylethylamine (4.47 mL, 25.6 mmol) is added and the reaction mixture is stirred for 1 hour. The methylene chloride solution of the distillate from the prior reaction is then added and the reaction mixture is stirred and maintained at −78° C. for 5 hours, then allowed to warm to ambient temperature overnight. The excess reagents are quenched by addition of 2N HCI (40 mL). Saturated aqueous ammonium chloride is added, and the mixture is extracted with CH₂Cl₂. The extract is dried over magnesium sulfate, filtered and evaporated. Silica chromatography (hexanes/ethyl acetate) separates the two diastereomeric products.

### EXAMPLE 4

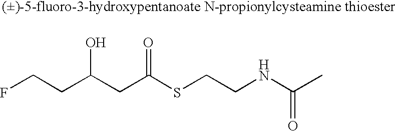

A 1.0 M solution of sodium methoxide in methanol (10 mL) is added to N,S-dipropionylcysteamine (18.9 g) under inert atmosphere and stirred until complete dissolution. After 30 minutes, acetic acid (0.4 mL) is added, and the resulting mixture is transferred by cannula to a flask containing (±)-N-(5-fluoro-3-hydroxypentanoyl)-2-benzoxazolone (25.3 g) under inert atmosphere. The mixture is stirred for 15 minutes, then is concentrated under vacuum. The residue is dissolved in ethyl acetate and washed sequentially with phosphate buffer, pH 6, and brine, then dried over magnesium sulfate, filtered, and evaporated. Silica gel chromatography (ethyl acetate/hexanes) yields the product. EXAMPLE 5

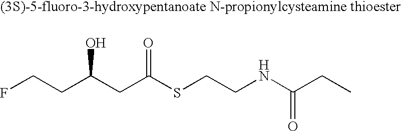

A 2.0 M solution of trimethylaluminum in hexanes (8.0 mL) is added to a stirred −10° C. solution of N-propionylcysteamine (2.5 g) in 200 mL of tetrahydrofuran. After 2 hours, a solution of (4R)-3-[(3S)-5-fluoro-3-hydroxypentanoyl)-4-isopropyl-2-oxazolidinone (0.9 g) in 100 mL of tetrahydrofuran is added and the mixture is allowed to stir at ambient temperature overnight. The mixture is acidified with 1 N HCl to pH 5 and extracted with ethyl acetate. The extract is washed sequentially with sat. aq. CuSO₄ and brine, dried over magnesium sulfate, filtered, and evaporated. The product is isolated by silica gel chromatography.

### EXAMPLE 6

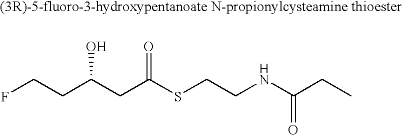

A 2.0 M solution of trimethylaluminum in hexanes (8.0 mL) is added to a stirred −10° C. solution of N-propionylcysteamine (2.5 g) in 200 mL of tetrahydrofuran. After 2 hours, a solution of (4R)-3-[(3R)-5-fluoro-3-hydroxypentanoyl)-4-isopropyl-2-oxazolidinone (0.9 g) in 100 mL of tetrahydrofuran is added and the mixture is allowed to stir at ambient temperature overnight. The mixture is acidified with 1 N HCl to pH 5 and extracted with ethyl acetate. The extract is washed sequentially with sat. aq. CuSO₄ and brine, dried over magnesium sulfate, filtered, and evaporated. The product is isolated by silica gel chromatography.

### EXAMPLE 7

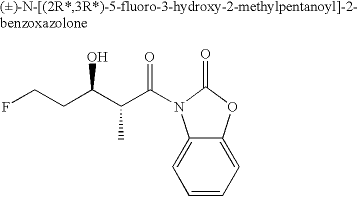

Step 1 o-Iodoxybenzoic acid (7.90 g, 28.2 mmol) was added to DMSO (19 mL) and stirred for 20 minutes until dissolved. 3-Fluoropropanol (2.0 g, 25.6 mmol) was added and the resulting mixture was stirred for 3 hours. The 3-fluoropropanal was distilled directly from the reaction vessel and condensed at −78° C. The distillate was dissolved in methylene chloride (10 mL) and used directly in the subsequent reaction.

Step 2. Chlorodicyclohexylborane (3.6 g) is added to a 0° C. solution of N-propionyl-2-benzoxazolone (2.7 g) in 150 mL of ether, followed by addition of dimethylethylamine (1.2 g). After stirring for 1 hour, the mixture is cooled to −78° C. and the above prepared solution of fluoropropanal is added. The mixture is stirred for 3 hours, then is allowed to warm to −20° C. over a 2-hour period prior to addition of 2:1 methanol/sat. aq. NH₄Cl (85 mL). The mixture is stirred for 5 minutes at 0° C., then poured into 250 mL of CH₂Cl₂ and 40 mL of water. The aqueous phase is separated and extracted with CH₂Cl₂ and the organic phases are combined, dried over sodium sulfate, filtered, and evaporated. The residue is dissolved in 300 mL of CH₂Cl₂ and stirred overnight with 100 mL of activated Amberlite IRA-743 resin (Hicks et al., Carbohydrate Research (1986) 147: 39-48). The mixture is filtered through a plug of silica gel using ethyl acetate to rinse, and the eluate is concentrated. The product is isolated by silica gel chromatography.

### EXAMPLE 8

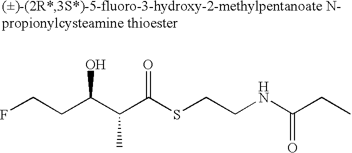

A 1.0 M solution of sodium methoxide in methanol (10 mL) is added to N,S-dipropionylcysteamine (18.9 g) under inert atmosphere and stirred until complete dissolution. After 30 minutes, acetic acid (0.4 mL) is added, and the resulting mixture is transferred by cannula to a flask containing (±)-(2R*,3S*)-N-(5-fluoro-3-hydroxy-2-methylpentanoyl)-2-benzoxazolone (26 g) under inert atmosphere. The mixture is stirred for 15 minutes, then is concentrated under vacuum. The residue is dissolved in ethyl acetate and washed sequentially with phosphate buffer, pH 6, and brine, then dried over magnesium sulfate, filtered, and evaporated. Silica gel chromatography (ethyl acetate/hexanes) yields the product.

### EXAMPLE 9

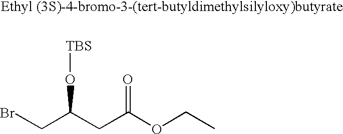

A solution of ethyl (3S)-4-bromo-3-hydroxybutyrate (21.0 g; Acros Chemicals) in 100 mL of CH₂Cl₂ is treated with tert-butyldimethylsilyl trifluoromethanesulfonate (30 g) and 2,6-lutidine (20 mL). After 1 hour, the mixture is washed sequentially with water and brine, dried over MgSO₄, filtered, and evaporated. The product silyl ether is purified by silica gel chromatography.

### EXAMPLE 10

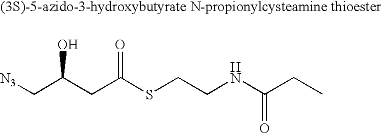

Step 1. A suspension of ethyl (3S)-4-bromo-3-(tert-butyldimethylsilyloxy)butyrate (32.5 g) and sodium azide (15 g) in 100 mL of dimethylformamide is heated at 60° C. for 1 hour. The mixture is cooled to ambient temperature, poured into 1000 mL of water, and extracted with ether. The extract is dried over MgSO₄, filtered, and evaporated to dryness under vacuum. The product azide is isolated by silica gel chromatography.

Step 2. A solution of ethyl (3S)-4-azido-3-(tert-butyldimethylsilyloxy)butyrate (28.7 g) in 1500 mL of THF and 300 mL of water is cooled to 0° C. and treated sequentially with 30% aqueous H₂O₂ (100 mL) and a 1.0 M solution of lithium hydroxide in water (200 mL). After stirring for 12 hours, the mixture is treated with aqueous sodium thiosulfate and concentrated to a slurry under vacuum. The slurry is carefully acidified to pH 4 using 1 N HCl, and extracted with CH₂Cl₂. The extract is dried over MgSO₄, filtered, and evaporated. The product acid is isolated by silica gel chromatography.

Step 3. A solution of(3S)-4-azido-3-(tert-butyldimethylsilyloxy)butyric acid (26 g) in 500 mL of THF is treated with diphenylphosphorylazide (30 g) and triethylamine (50 mL) for one hour. N-propionylcysteamine (15 g) is added, and the mixture is stirred overnight. The solution is concentrated, and the residue is dissolved in ethyl acetate and washed sequentially with water, 1 N HCl, sat. aq. CuSO₄, and brine, then dried over MgSO₄, filtered, and evaporated. The product thioester is purified by silica gel chromatography.

Step 4. A solution of(3S)-4-azido-3-(tert-butyldimethylsilyloxy)butyrate N-propionylcysteamine thioester (37.5 g) in 1500 mL of acetonitrile and 300 mL of water is treated with 48% hydrofluoric acid (150 mL) for 2 hours at ambient temperature. A second portion of 48% HF (150 mL) is added, and the reaction is continued an additional 2 hours. The reaction is carefully treated with sat. NaHCO₃ to neutralize excess HF. The mixture is concentrated under vacuum, and the residue is diluted with ethyl acetate, washed sequentially with water and brine, dried over MgSO_{4,} filtered, and evaporated. The product is isolated by silica gel chromatography.

### EXAMPLE 11

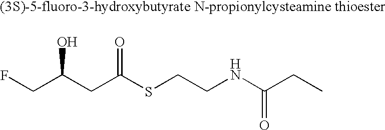

Step 1. Ethyl 4-fluoroacetoacetate is reduced to ethyl (3S)-4-fluoro-3-hydroxybutyrate using yeast as described in K. Tanida & Y. Suzuki, “Optically active fluorine-containing 3-hydroxybutyric acid esters and processes for producing same,” European Patent Application No. 427396.

Step 2. A solution of ethyl (3S)-4-fluoro-3-hydroxybutyrate (15.0 g) in 100 mL of CH₂Cl₂ is treated with tert-butyldimethylsilyl trifluoromethanesulfonate (30 g) and 2,6-lutidine (20 mL). After 1 hour, the mixture is washed sequentially with water and brine, dried over MgSO₄, filtered, and evaporated. The product silyl ether is purified by silica gel chromatography.

Step 3. A solution of ethyl (3S)-4-fluoro-3-(tert-butyldimethylsilyloxy)butyrate (26.4 g) in 1500 mL of THF and 300 mL of water is cooled to 0° C. and treated sequentially with 30% aqueous H₂O₂ (100 mL) and a 1.0 M solution of lithium hydroxide in water (200 mL). After stirring for 12 hours, the mixture is treated with aqueous sodium thiosulfate and concentrated to a slurry under vacuum. The slurry is carefully acidified to pH 4 using 1 N HCl, and extracted with CH₂Cl₂. The extract is dried over MgSO₄, filtered, and evaporated. The product acid is isolated by silica gel chromatography.

Step 4. A solution of (3S)-4-fluoro-3-(tert-butyldimethylsilyloxy)butyric acid (23.6 g) in 500 mL of THF is treated with diphenylphosphorylazide (30 g) and triethylamine (50 mL) for one hour. N-propionylcysteamine (15 g) is added, and the mixture is stirred overnight. The solution is concentrated, and the residue is dissolved in ethyl acetate and washed sequentially with water, 1 N HCl sat. aq. CuSO₄, and brine, then dried over MgSO₄, filtered, and evaporated. The product thioester is purified by silica gel chromatography.

Step 5. A solution of (3S)-4-fluoro-3-(tert-butyldimethylsilyloxy)butyrate N-propionylcysteamine thioester (35 g) in 1500 mL of acetonitrile and 300 mL of water is treated with 48% hydrofluoric acid (150 mL) for 2 hours at ambient temperature. A second portion of 48% HF (150 mL) is added, and the reaction is continued an additional 2 hours. The reaction is carefully treated with sat. NaHCO₃ to neutralize excess HF. The mixture is concentrated under vacuum, and the residue is diluted with ethyl acetate, washed sequentially with water and brine, dried over MgSO_{4,} filtered, and evaporated. The product is isolated by silica gel chromatography.

### EXAMPLE 12

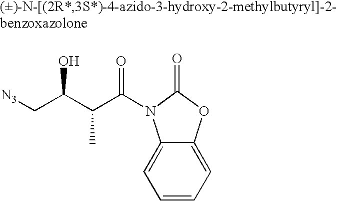

Step 1. Chlorodicyclohexylborane (3.6 g) is added to a 0° C. solution of N-propionyl-2-benzoxazolone (2.7 g) in 150 mL of ether, followed by addition of dimethylethylamine (1.2 g). After stirring for 1 hour, the mixture is cooled to −78° C. and a 1 M solution of chloroacetaldehyde in ether (20 mL) is added. The mixture is stirred for 3 hours, then is allowed to warm to −20° C. over a 2-hour period prior to addition of 2:1 methanol/sat. aq. NH₄Cl (85 mL). The mixture is stirred for 5 minutes at 0° C., then poured into 250 mL of CH₂Cl₂ and 40 mL of water. The aqueous phase is separated and extracted with CH₂Cl₂ and the organic phases are combined, dried over sodium sulfate, filtered, and evaporated. The residue is dissolved in 300 mL of CH₂Cl₂ and stirred overnight with 100 mL of activated Amberlite IRA-743 resin (Hicks et al., Carbohydrate Research 147: 39-48 (1986)). The mixture is filtered through a plug of silica gel using ethyl acetate to rinse, and the eluate is concentrated. The product is isolated by silica gel chromatography.

Step 2. The above product is dissolved in 10 mL of dimethylformamide and treated with sodium azide (5 g) at 60° C. for 1 hour. The mixture is cooled to ambient temperature, poured into water, and extracted with ethyl acetate. The extract is washed sequentially with 1 N HCl, sat. NaHCO₃, and brine, then dried over MgSO₄, filtered, and evaporated. The product is isolated by silica gel chromatography.

### EXAMPLE 13

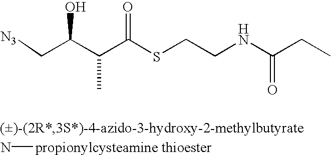

A 1.0 M solution of sodium methoxide in methanol (10 mL) is added to N,S-dipropionylcysteamine (18.9 g) under inert atmosphere and stirred until complete dissolution. After 30 minutes, acetic acid (0.4 mL) is added, and the resulting mixture is transferred by cannula to a flask containing (±)-N-[(2R*,3S*)-4-azido-3-hydroxy-2-methylbutyryl]-2-benzoxazolone (26 g) under inert atmosphere. The mixture is stirred for 15 minutes, then is concentrated under vacuum. The residue is dissolved in ethyl acetate and washed sequentially with phosphate buffer, pH 6, and brine, then dried over magnesium sulfate, filtered, and evaporated. Silica gel chromatography (ethyl acetate/hexanes) yields the product.

### EXAMPLE 14

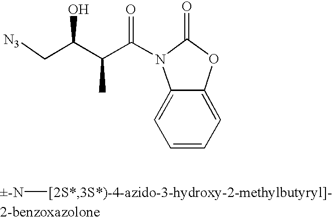

Step 1. Titanium tetrachloride (20.8 g) is added to a 0° C. solution of N-propionyl-2-benzoxazolone (19.2 g) in 200 mL of CH₂Cl₂, followed by addition of triethylamine (16.8 mL). After stirring for 1 hour, a 1 M solution of chloroacetaldehyde in ether (120 mL) is added. The mixture is stirred for 3 hours, and then quenched by addition of 500 mL of 2N HCl. The phases are separated, and the organic phase is filtered through a pad of silica gel. The pad is washed with ether, and the eluents are combined and evaporated. The product is isolated by silica gel chromatography.

Step 2. The above product is dissolved in 100 mL of dimethylformamide and treated with sodium azide (50 g) at 60° C. for 1 hour. The mixture is cooled to ambient temperature, poured into water, and extracted with ethyl acetate. The extract is washed sequentially with 1 N HCl, sat. NaHCO₃, and brine, then dried over MgSO₄, filtered, and evaporated. The product is isolated by silica gel chromatography.

### EXAMPLE 15

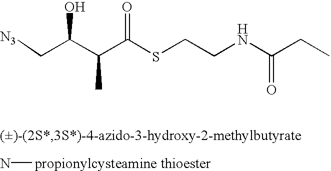

A 1.0 M solution of sodium methoxide in methanol (10 mL) is added to N,S-dipropionylcysteamine (18.9 g) under inert atmosphere and stirred until complete dissolution. After 30 minutes, acetic acid (0.4 mL) is added, and the resulting mixture is transferred by cannula to a flask containing (±)-N-[(2S*,3S*)-4-azido-3-hydroxy-2-methylbutyryl]-2-benzoxazolone (26 g) under inert atmosphere. The mixture is stirred for 15 minutes, then is concentrated under vacuum. The residue is dissolved in ethyl acetate and washed sequentially with phosphate buffer, pH 6, and brine, then dried over magnesium sulfate, filtered, and evaporated. Silica gel chromatography (ethyl acetate/hexanes) yields the product.

### EXAMPLE 16

S. fradiae Clean Host

This example describes the protocol for making a clean host in S. fradiae where the PKS genes (tylG ORF1 to tylG ORF5) are deleted via double cross-over homologous recombination. In one embodiment, the clean host is made from S. fradiae Russia-99 that makes high tylosin in large amounts. The non-PKS genes in the PKS cluster are intact and available to act on the modified PKS product. In another embodiment, the clean host is made from S. fradiae NRRL 12170, a mutant strain that makes DMT. Consequently, the expression of a recombinant PKS gene in this host results in a corresponding DMT derivative because one or more functions necessary for adding deoxyallose at the hydroxymethyl at C-14 is unavailable. In another embodiment, the clean host is made from KA-427-261, a mutant strain of S. fradiae that makes only tylactone. KA-427-261 is described by Omura et al., J. Antibiot. 33: 915 (1980) which is incorporated herein by reference. Consequently, expression of a recombinant PKS gene in this host yields the unmodified PKS product. The following method can be used to make a clean host from any strain of S. fradiae and is not limited to the three strains discussed above.

Two DNA fragments are generated using polymerase chain reaction (“PCR”), one corresponding to the DNA sequence to the left of the tylG region, and the other corresponding to the DNA sequence to the right of tylG. The two fragments are cloned next to each other on a suicide vector (a vector that will not replicate in S. fradiae) that carries a selectable antibiotic resistance marker that works in S. fradiae. One example of such a marker is the apramycin resistance gene aacCIV. The vector is then introduced into S. fradiae (e.g., by conjugation from E. coli), and apramycin-resistant colonies are selected and isolated. These isolates correspond to recombinants where the vector has integrated into the chromosome by a single homologous cross-over event either through the interval to the left of tylG, or to the right of tylG (and usually confirmed by PCR or Southern blotting). The desired mutant is obtained by propagating one of the apramycin-resistant isolates in the absence of apramycin for one or more generations, and then screening single colonies by replica plating for those that are susceptible to apramycin. The apramycin-susceptible isolates will either be wild-type (wherein the vector was excised out in the same manner that it went in), or mutant (wherein the vector integrated through one interval in the first homologous recombination step, and was excised out through the other interval in the second homologous recombination step). The wild-type and the mutant colonies can be distinguished by checking production of tylosin, and by PCR or Southern blotting.

### EXAMPLE 17

S. ambofaciens Clean Host

This example describes the protocol for making a clean host in S. ambofaciens where the PKS genes (srmG ORF1 to srmG ORF5) are deleted via double cross-over homologous recombination. In one embodiment, the clean host is made from a spiramycin producer ATCC 15154. The non-PKS genes in the PKS cluster are intact and available to act on the modified PKS product to yield a corresponding spiramycin derivative. In another embodiment, the clean host is made from a strain described by Omura et al., Chem. Pharm. Bull. 27: 176 (1979) which is incorporated herein by reference. This latter strain is a block mutant that can only make platenolide. As a result, expression of a recombinant PKS in this host yields the unmodified PKS product. The following method can be used to make a clean host from any strain of S. ambofaciens and is not limited to the two strains discussed above.

Two DNA fragments are generated using polymerase chain reaction (“PCR”), one corresponding to the DNA sequence to the left of the srmG region, and the other corresponding to the DNA sequence to the right of srmG. The two fragments are cloned next to each other on a suicide vector (a vector that will not replicate in S. ambofaciens) that carries a selectable antibiotic resistance marker that works in S. ambofaciens. One example of such a marker is the apramycin resistance gene aacCIV. The vector is then introduced into S. ambofaciens (e.g., by conjugation from E. coli), and apramycin-resistant colonies are selected and isolated. These isolates correspond to recombinants where the vector has integrated into the chromosome by a single homologous cross-over event either through the interval to the left of srmG, or to the right of srmG (and usually confirmed by PCR or Southern blotting). The desired mutant is obtained by propagating one of the apramycin-resistant isolates in the absence of apramycin for one or more generations, and then screening single colonies by replica plating for those that are sensitive to apramycin. The apramycin-sensitive isolates will either be wild-type (wherein the vector was excised out in the same manner that it went in), or mutant (wherein the vector integrated through one interval in the first homologous recombination step, and was excised out through the other interval in the second homologous recombination step). The wild-type and the mutant colonies can be distinguished by checking production of spiramycin (or in the case of the block mutant strain, platenolide), and by PCR or Southern blotting.

### EXAMPLE 18

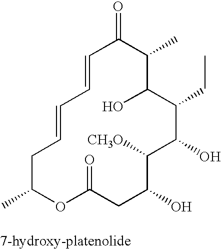

This example describes methods for making 7-hydroxy-platenolide. In the normal course of biosynthesis, module 5 binds ethylmalonyl CoA as the extender unit; extends the growing polyketide product by two carbons from the condensation of the ethylmalonyl CoA; and reduces the β-ketone of the previously added two-carbon unit to a methylene group. The 7-hydroxy-platenolide is made by eliminating the activities of the dehydratase and enoylreductase and expressing the modified PKS gene in a suitable host that do not also possess post-PKS modification enzymes. A suitable host is derived from a blocked mutant of S. ambofaciens that makes platenolide as described in Example 17.

In one method, a point mutation is made in the DH gene that alters the active site histidine into another amino acid such as leucine. This change would effectively turn domain 5 into one that only possesses ketoreductase activity. If KS6 is unable to recognized the modified acyl chain, that KS can also be replaced with one that normally processes the (α-ethyl, β-hydroxyl substrate such as the KS2 of the nystatin PKS. This latter construct is expected to make the 7(+)-hydroxyplatenolide. If the 7(−)-hydroxyplatenolide is desired, then KS6 of the platenolide PKS can be replaced with a KS that normally processes the hydroxyl of the opposite stereochemistry such as the KS1 of the nystatin PKS.

In another method, the DH/ER/KR domains of the platenolide PKS is replaced with a KR domains such. as KR1 of the nystatin PKS and the KR8 of the rifamycin PKS. If KS6 is unable to recognized the modified acyl chain, that KS can also be replaced with one that normally processes the α-ethyl, β-hydroxyl substrate such as the KS2 of the nystatin PKS. This latter construct is expected to make the 7(+)-hydroxyplatenolide. If the 7(−)-hydroxyplatenolide is desired, then KS6 of the platenolide PKS can be replaced with a KS that normally processes the hydroxyl of the opposite stereochemistry such as the KS1 of the nystatin PKS.

Either stereoisomer of 7-hydroxy-platenolide can be modified using hybrid biosynthesis or bioconversion. The 7-hydroxy platenolide can be fed to strains that are grown in the presence of cerulenin and that normally make a platenolide-based as well as non-platenolide-based macrolides for additional post-PKS modifications at the unaffected positions. For example, when added to a pikromycin strain grown in the presence of cerulenin, 7-hydroxy-5-desosaminyl-platenolide is made. When added to a midecamycin producing strain of S. mycarofaciens that is grown in the presence of cerulenin, 7-hydroxy-midecamycins (A₁, A₂, A₃, and A4) are made. When added to a spiramycin producing strain of S. ambofaciens that is grown in the presence of cerulenin, 7-hydroxy-spiramycins (I, II, III, IV, V, and V) are made.

### EXAMPLE 19

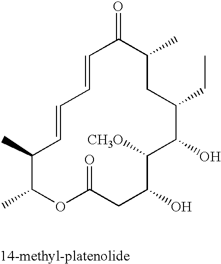

This example describes methods for making 14-methyl-platenolide. Unlike a tylactone PKS, ATl of a platenolide PKS specifies a malonyl extender unit instead of a methylmalonyl extender unit. This can be accomplished by substituting the AT1, the domains (AT1-DH1-ER1-KR1-ACP1-KS2), or the ORF1 of the platenolide PKS with the corresponding AT, domains, or ORF1 from the tylactone PKS and expressing the construct in a suitable host that do not possess post-PKS modification enzymes. A suitable host is derived from a blocked mutant of S. ambofaciens that makes platenolide as described in Example 17. Alternatively, 14-methyl and 14-hydroxymethyl platenolide can be made using chemobiosynthesis as shown by Scheme A.

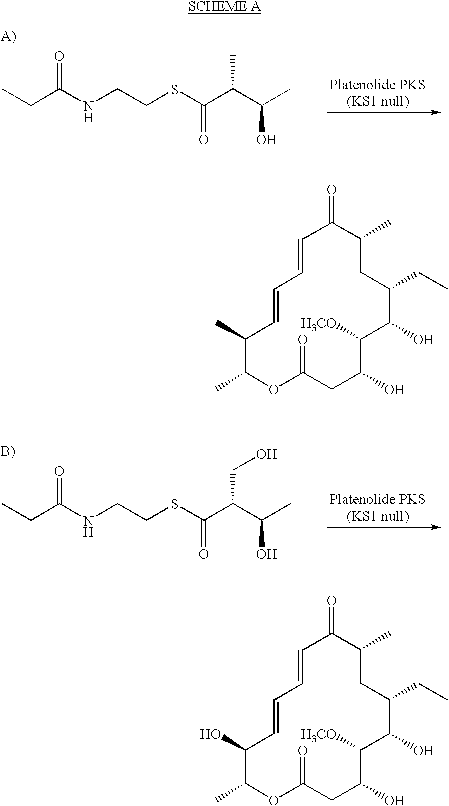

The 14-methyl-platenolide can then be modified using hybrid biosynthesis using any of the strains listed in Table 2. Because platenolide-based macrolides do not usually have a methyl group at C-14, use of these strains to bioconvert 14-methyl-platenolide will result in the corresponding 14-methyl macrolide derivative. In contrast, tylactone-based macrolides do have a methyl group at C-14 which can be further modified. For example, using S. fradiae to bioconvert will result in 4-desmethyl-4-methoxy-12-desmethyl-15-desethyl-15-methyl tylosin. Using DMT producing strain S. fradiae NRRL 12170 to bioconvert 14-methyl-platenolide will result in

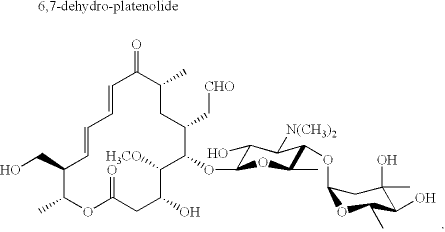

This example describes methods for making 6,7-dehydro-platenolide. In the normal course of biosynthesis, module 5 binds ethylmalonyl CoA as the extender unit; extends the growing polyketide product by two carbons from the condensation of the ethylmalonyl CoA; and reduces the β-ketone of the previously added two-carbon unit to a methylene group. The 6,7-dehydro-platenolide is made by eliminating the activity of the enoylreductase and expressing the modified PKS gene in a suitable host that do not also possess post-PKS modification enzymes. In one method, the ER activity is eliminated by introducing a mutation in the PKS gene to alter the active site GG (glycine-glycine) residues to an AP (alanine-proline) or an AS (alanine-serine). A suitable host is derived from a blocked mutant of S. ambofaciens that makes platenolide as described in Example 17.

### EXAMPLE 21

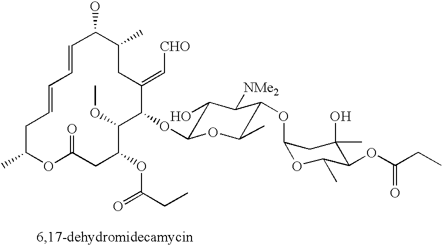

A solution of 6,7-dehydromidecamycin (812 mg) in 10 mL of anhydrous CH₂Cl₂ is treated with glacial acetic acid (100 mg) at ambient temperature. After 20 standing overnight, the mixture is poured into sat. aq. NaHCO₃. The organic phase is washed with brine, dried over sodium sulfate, filtered, and evaporated. The product is purified by silica gel chromatography.

### EXAMPLE 22

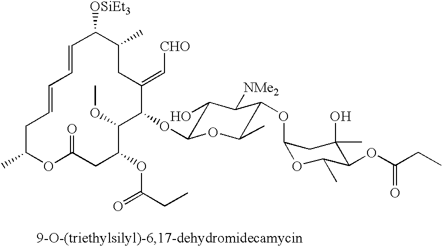

A solution of6,17-dehydromidecamycin (812 mg) in 10 mL of anhydrous pyridine is treated with chlorotriethylsilane (200 mg) for 12 hours at ambient temperature. The mixture is evaporated to dryness, then partitioned between CH₂Cl₂ and water. The organic phase is washed with brine, dried over sodium sulfate, filtered, and evaporated. The product is purified by silica gel chromatography.

### EXAMPLE 23

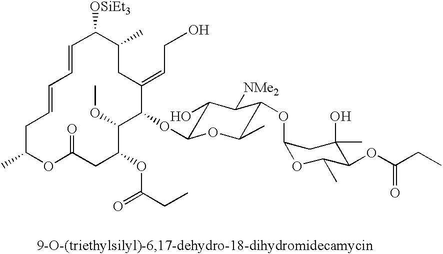

Sodium borohydride (38 mg) is added to a −78° C. solution of 9-O-(triethylsilyl)-6,17-dehydromidecamycin (930 mg) in 40 mL of methanol containing cerium trichloride hexahydrate (365 mg). After 15 minutes, the mixture is diluted with acetone, and the mixture is warmed to ambient temperature and concentrated to dryness.

The residue is dissolved in ethyl acetate and washed with sat. aq. NaHCO₃ followed by brine. The solution is dried over sodium sulfate, filtered, and evaporated. The product is purified by silica gel chromatography.

### EXAMPLE 24

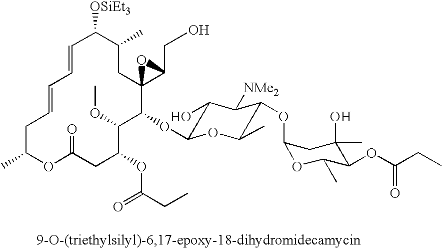

Titanium tetraisopropoxide (340 mg) is added to a solution of (−)-diisopropyl tartrate (330 mg) in 10 mL of CH₂Cl₂, and the mixture is cooled to −20° C. and treated with a 5 M solution of tert-butylhydroperoxide in decane (0.80 mL) followed by a solution of 9-O-(triethylsilyl)-6,17-dehydro-18-dihydromidecamycin (930 mg). The mixture is kept at −20° C. for 24 hours, then is quenched by addition of dimethyl sulfide, diluted with CH₂Cl₂, and washed successively with sat. aq. NaF, sat. aq. NaHCO₃, and brine. The solution is dried over sodium sulfate, filtered, and evaporated. The product is isolated by rapid chromatography on silica gel.

### EXAMPLE 25

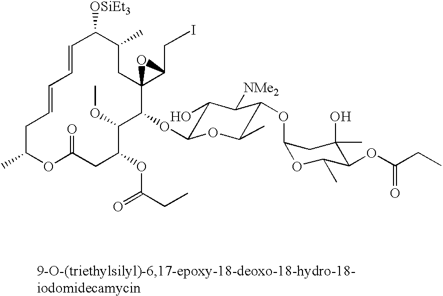

A solution of 9-O-(triethylsilyl)-6,17-epoxy-18-dihydromidecamycin (950 mg), triphenylphosphine (922 mg), and imidazole (275 mg) in 30 mL of benzene and 60 mL of ether is treated with iodine (760 mg) in one portion with vigorous stirring. After 2 hours, the mixture is poured into ether and washed successively with water, sat. aq. NaHCO₃, and brine. The solution is dried over sodium sulfate, filtered, and evaporated. The product is isolated by rapid chromatography on silica gel.

### EXAMPLE 26

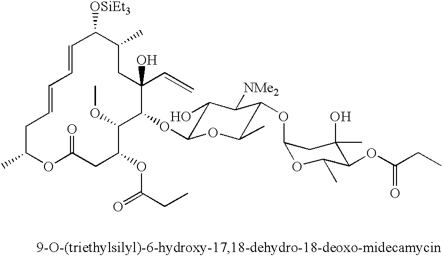

A solution of 9-O-(triethylsilyl)-6,17-epoxy-18-deoxo-18-hydro-18-iodomidecamycin (1.0 g) in 10 mL of tetrahydrofuran is added to a suspension of zinc dust (200 mg) in 1 mL of sat. aq. NH₄Cl. The mixture is stirred vigorously overnight, then diluted with ethyl acetate and filtered. The filtrate is washed successively with sat. aq. NaHCO₃, and brine. The solution is dried over sodium sulfate, filtered, and evaporated. The product is isolated by chromatography on silica gel.

### EXAMPLE 27

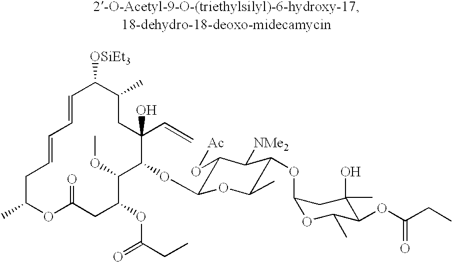

A solution of 9-O-(triethylsilyl)-6-hydroxy-17,18-dehydro-18-deoxo-midecamycin (930 mg) in 10 mL of CH₂Cl₂ is treated with acetic anhydride (200 mg). The mixture is stirred for 2 hours, then evaporated. The residue is dissolved in ethyl acetate and washed successively with sat. aq. NaHCO₃, and brine. The solution is dried over sodium sulfate, filtered, and evaporated. The product is isolated by chromatography on silica gel.

### EXAMPLE 28

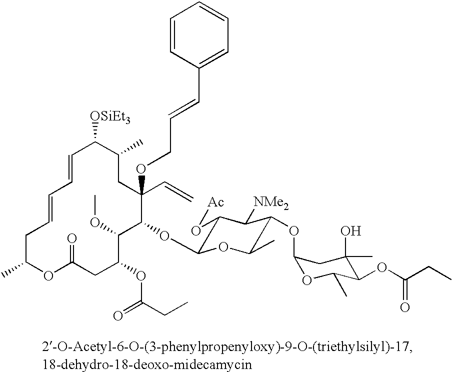

A solution of2′-O-acetyl-9-O-(triethylsilyl)-6-hydroxy-17,18-dehydro-18-deoxo-midecamycin (970 mg) and β-bromostyrene (400 mg) in 10 mL of tetrahydrofuran and 1 mL of methylsulfoxide is cooled to 0° C. and treated dropwise with a 1 M solution of potassium tert-butoxide in tetrahydrofuran (3 mL) over 2 hours. The mixture is stirred for an additional 1 hour, then is poured into sat. aq. NaHCO₃. The mixture is extracted with ethyl acetate, and the extract is washed with brine, dried over sodium sulfate, filtered, and evaporated. The product is isolated by chromatography on silica gel.

### EXAMPLE 29

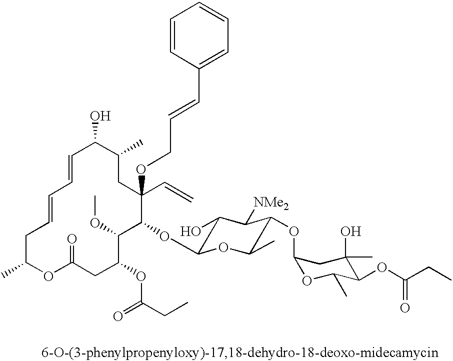

A solution of 2′-O-Acetyl-6-O-(3-phenylpropenyloxy)-9-O-(triethylsilyl)-17,18-dehydro-18-deoxo-midecamycin (1.0 g) in 10 mL of tetrahydrofuran is treated with a 1 M solution of tetrabutylammonium fluoride in tetrahydrofuran (2 mL) for 12 hours at ambient temperature. The mixture is evaporated and the residue is dissolved in 10 mL of methanol and kept for 12 hours. The mixture is evaporated, the residue is dissolved in ethyl acetate, and the solution is washed successively with water, sat. aq. NaHCO₃, and brine, then dried over sodium sulfate, filtered, and evaporated. The product is purified by silica gel chromatography.

### EXAMPLE 30

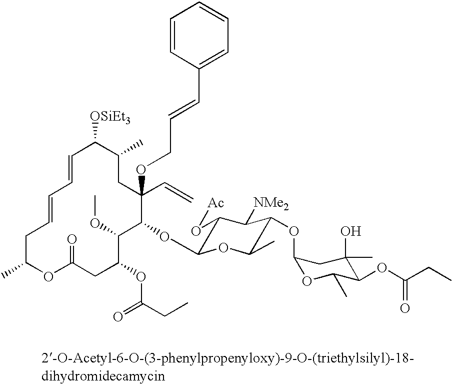

A solution of 2′-O-Acetyl-6-O-(3-phenylpropenyloxy)-9-O-(triethylsilyl)-17,18-dehydro-18-deoxo-midecamycin (1.0 g) in 10 mL of tetrahydrofuran is treated with a 1 M solution of diethylborane (1 mL) at 0° C. for 1 hour, then then mixture is kept for 12 hours at ambient temperature. Hydrogen peroxide (1 mL of 30% aqueous solution) is added, and the mixture is stirred for 1 hour. The mixture is diluted with ethyl acetate, and the solution is washed successively with water, sat. aq. NaHCO₃, and brine, then dried over sodium sulfate, filtered, and evaporated. The product is purified by silica gel chromatography.

### EXAMPLE 31

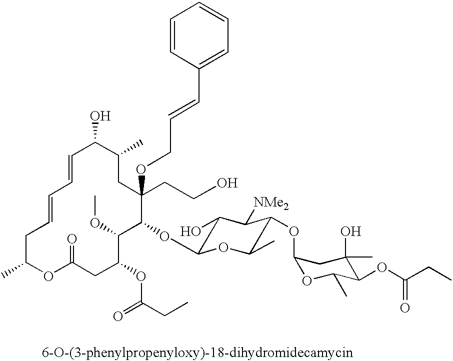

A solution of2′-O-Acetyl-6-O-(3-phenylpropenyloxy)-9-O-(triethylsilyl)-18-dihydromidecamycin (1.0 g) in 10 mL of tetrahydrofuran is treated with a 1 M solution of tetrabutylammonium fluoride in tetrahydrofuran (2 mL) for 12 hours at ambient temperature. The mixture is evaporated and the residue is dissolved in 10 mL of methanol and kept for 12 hours. The mixture is evaporated, the residue is dissolved in ethyl acetate, and the solution is washed successively with water, sat. aq. NaHCO₃, and brine, then dried over sodium sulfate, filtered, and evaporated. The product is purified by silica gel chromatography.

### EXAMPLE 32

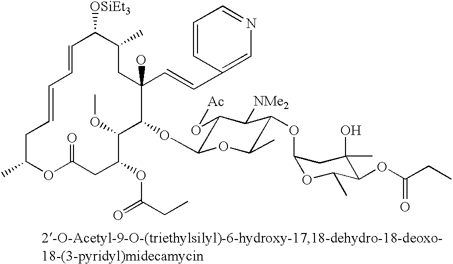

A mixture of 2 ′-O-acetyl-9-O-(triethylsilyl)-6-hydroxy-17,18-dehydro-18-deoxo-midecamycin (970 mg), 3-bromopyridine (200 mg), tris(dibenzylidenacetone)-dipalladium•CHCl₃ (100 mg), tri(o-tolyl)phosphine (300 mg), and triethylamine (200 mg) in 10 mL of degassed acetonitrile is heated at 80° C. for 3 days. The mixture is evaporated, and the residue is dissolved in ethyl acetate and washed with sat. aq. NaHCO₃ and brine. The solution is dried over sodium sulfate, filtered, and evaporated. The product is purified by silica gel chromatography.

### EXAMPLE 33

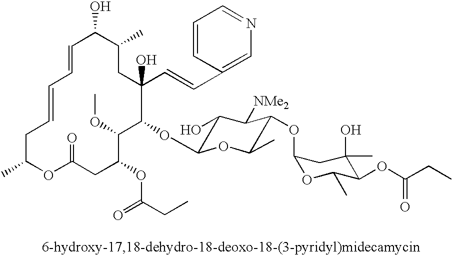

A solution of 2′-O-Acetyl-9-O-(triethylsilyl)-6-hydroxy-17,18-dehydro-18-deoxo-18-(3-pyridyl)midecamycin (1.0 g) in 10 mL of tetrahydrofuran is treated with a 1 M solution of tetrabutylammonium fluoride in tetrahydrofuran (2 mL) for 12 hours at ambient temperature. The mixture is evaporated and the residue is dissolved in 10 mL of methanol and kept for 12 hours. The mixture is evaporated, the residue is dissolved in ethyl acetate, and the solution is washed successively with water, sat. aq. NaHCO₃, and brine, then dried over sodium sulfate, filtered, and evaporated. The product is purified by silica gel chromatography.

### EXAMPLE 34

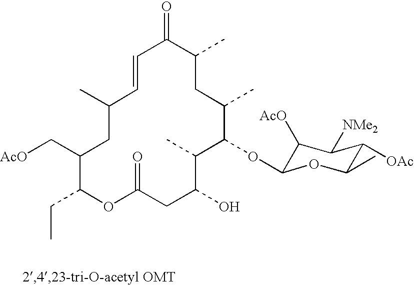

To 5.31 g OMT in 50 m L ethyl acetate, was added acetic anhydride (2.9 mL) and K₂CO₃ (5g). The mixture was stirred at room temperature for 20 hour. The reaction was filtered to remove K₂CO₃ followed by addition of 300 mL ethyl acetate. The organic material was washed with sat. NaHCO₃ (100 mL), dried over Na₂SO₄, filtered, and evaporated to dryness. The product (1.9 g) was isolated by silica gel column chromatography (5% acetone in hexane to 25% acetone in hexane in the presence of 2% triethyl amine).

### EXAMPLE 35

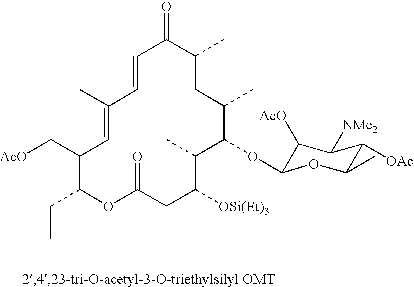

To 3, 2′.4′.23-tri-O-acetyl OMT (1.9 g) in 30 mL dichloromethane was added triethylamine (9.49 mL), DMAP (169 mg), and TESCl (4.6 mL). The mixture was stirred at room temperature for 19 hours. Ethyl acetate (300 mL) was added and the organic layer was washed with sat. NaHCO₃, dried over Na₂SO₄, filtered, and evaporated to dryness. The product (1.485 g) was obtained after purification by silica gel column (5% acetone in hexane to 10% acetone in hexane in the presence of 2% triethylamine).

### EXAMPLE 36

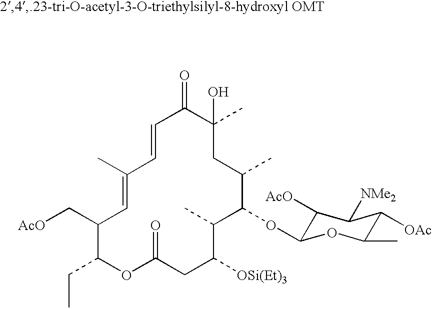

To 2′.4′.23-tri-O-acetyl-3-O-triethylsilyl OMT (1.485 g) in ehtyl acetate (60 mL), was added N-methyl-N-(trimethlsilyl)-trifluoroacetamide (1.69 mL) and ammonium iodide (53 mg). The reaction was kept at 85° C. for 12 hours. After the reaction was done, 300 mL of ethyl acetate was added and washed with sat. NaHCO₃ (2×80 mL). The organic layer was dried over Na₂SO₄, filtered, and evaporated to dryness. The residue was further dried under high vacuum for 1.5 hours. The product was used for next step without further purification.

To the above reaction product, was added 30 mL hexane at −20° C. Stirred at −20° C. for 5 minutes followed by addition of MCPBA (494 mg, ˜70% pure). The reaction mixture was stirred at −20° C. for another 15 minutes, then room temperature for 18 hours. After that, 150 mL of benzene was added. The organic was washed with sat. NaHCO₃ (2×50 mL), dried over Na₂SO₄, filtered, and evaporated to dryness. Separation was carried out on ISCO MPLC using a silica gel column to give 600 mg product.

### EXAMPLE 37

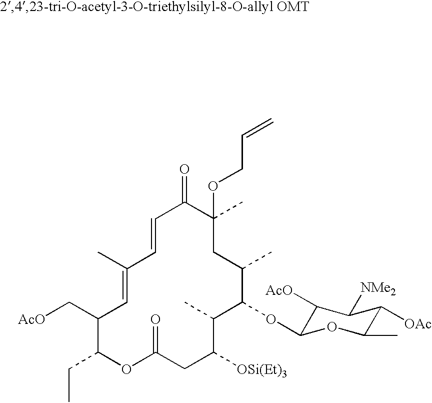

2′,4′,23-tri-O-acetyl-3-O-triethylsilyl-8-hydroxyl OMT (35.4 mg) was dissolved in 1.5 mL dry THF and 0.6 mL dry DMF. Freshly distilled allyl bromide (0.3 mL) was added and the reaction was stirred at room temperature. Sodium hydride (10 mg, 60% in mineral) was added and the reaction was stirred at room temperature for one hour. Ethyl acetate (20 mL) was added and the organic was washed with sat. NaHCO₃ (3×20 mL), dried over Na₂SO₄, filtered, and evaporated to dryness. The product (24.4 mg) was isolated by silica gel column(5% acetone in hexane to 10% acetone in hexane in the presence of 2% triethylamine).

### EXAMPLE 38

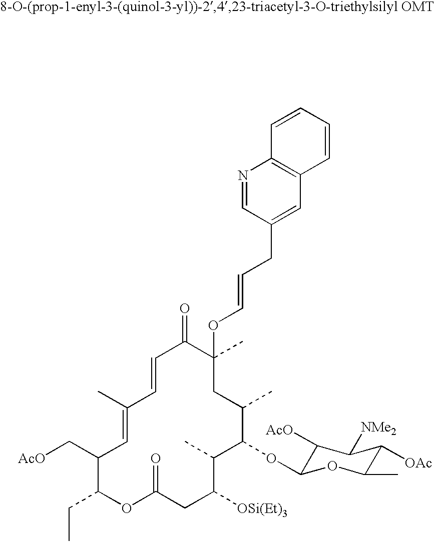

A mixture of 2′.4′.23-tri-O-acetyl-3-O-triethylsilyl-8-O-allyl OMT (27 mg), (o-tol)₃P (9.5 mg), Pd₂dBa₃.CHCl₃ (16.2 mg), triethylamine (8.7 μL), 3-bromoquinoline (21.5 □L) in 1 mL acetonitrile was kept at 90° C. for 24 hours. Ethyl acetate (15 mL) was added and the organic was washed with sat. NaHCO₃ (2×5 mL), dried over Na₂SO₄, filtered, and evaporated to dryness. The product (17 mg) was isolated by silica gel column(5% acetone in hexane to 10% acetone in hexane in the presence of 2% triethylamine).

### EXAMPLE 39

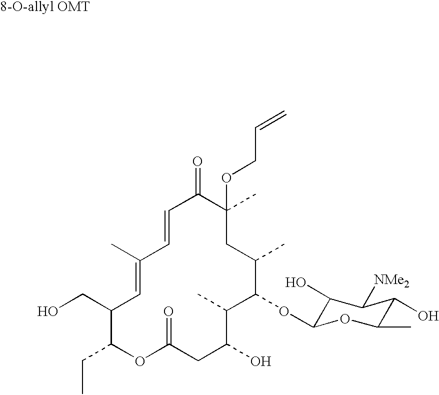

To 2′.4′.23-tri-O-acetyl-3-O-triethylsilyl-8-O-allyl OMT (4 mg) in THF was added TBAF (7 μL, 1 mM in THF). Stirred at room temperature for 30 minutes. Ethyl acetate (20 mL) was added and the organic was washed with sat. NaHCO₃ (3×20 mL), dried over Na₂SO₄, filtered, and evaporated to dryness. The residue was dissolved in 1 mL of methanol and triethylamine (70 μL ) was added. The reaction was kept at 70° C. overnight. After the solvent was removed under reduced pressure, the product (2.0 mg) was isolated by silica gel column(2% methanol in dichloromethane to 8% methanol in dichloromethane in the presence of 2% triethylamine).

### EXAMPLE 40

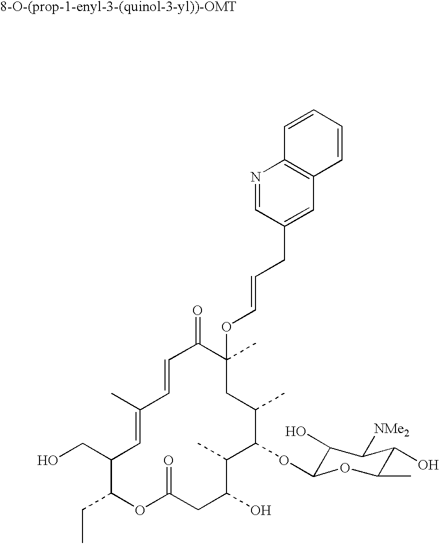

To 8-O-(prop-1-enyl-3-(quinol-3-yl))-2′,4′,23-triacetyl-3-O-triethylsilyl OMT (15.6 mg) in THF was added TBAF (21 μL, 1 mM in THF). Stirred at room temperature for 30 minutes. Ethyl acetate (20 mL) was added and the organic was washed with sat. NaHCO₃ (3×20 mL), dried over Na₂SO₄, filtered, and evaporated to dryness. The residue was dissolved in 1.6 mL of methanol and triethylamine (156 μL) was added. The reaction was kept at 70° C. overnight. After the solvent was removed under reduced pressure, the product (8.0 mg) was isolated by silica gel column(2% methanol in dichloromethane to 8% methanol in dichloromethane in the presence of 2% triethylamine).

### EXAMPLE 41

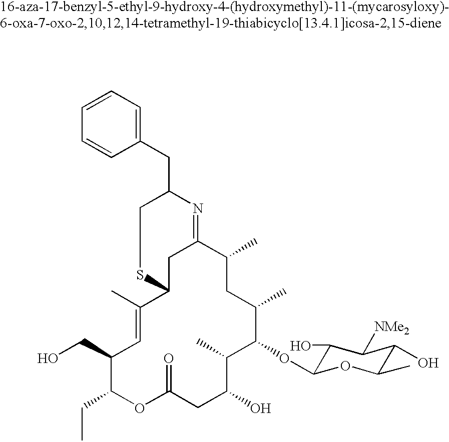

A mixture of 19-deformyl-5-O-mycarosyltylonolide (570 mg) and 2-amino-3-phenylpropanethiol (250 mg) in 10 mL of triethylamine is heated at reflux for 4 hours under inert atmosphere. The mixture is concentrated under reduced pressure, and the residue is purified by silica gel chromatography to provide the noncyclized amino ketone. The amino ketone (1 mmol) is dissolved in 10 mL of ethanol and treated with acetic acid (2 mmol) at ambient temperature for 16 hours. The solvent is evaporated, and the residue is dissolved in water. The pH is adjusted to 9 by addition of 1N NaOH, and the mixture is extracted with ethyl acetate. The extract is washed with brine, dried over MgSO₄, filtered, and evaporated. The product is isolated by silica gel chromatography.

### EXAMPLE 42

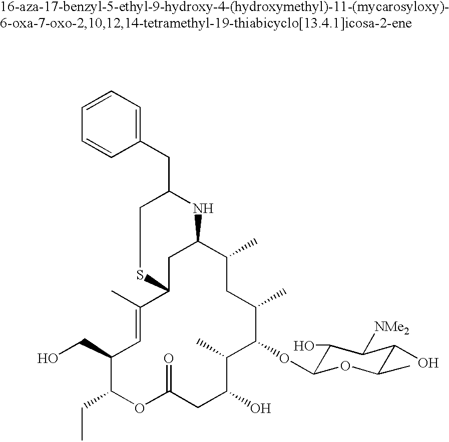

Titanium trichloride (20% aqueous solution, 1.7 mL) is added over a period of 1 hour to a solution of 16-aza-17-benzyl-5-ethyl-9-hydroxy-4-(hydroxymethyl)-11-(mycarosyloxy)-6-oxa-7-oxo-2, 10,12,1 4-tetramethyl-19-thiabicyclo[13.4.1]icosa-2,15-diene (718 mg), sodium cyanoborohydride (0.2 g), and ammonium acetate (1 g) in 15 mL of methanol. The mixture is stirred overnight at ambient temperature, then is diluted with water and washed with CH₂Cl₂. The aqueous phase is adjusted to pH 9.5 using IN NaOH and is extracted with CH₂Cl₂. The extract is dried over sodium sulfate, filtered, and evaporated. The product is purified by silica gel chromatography.

### EXAMPLE 43

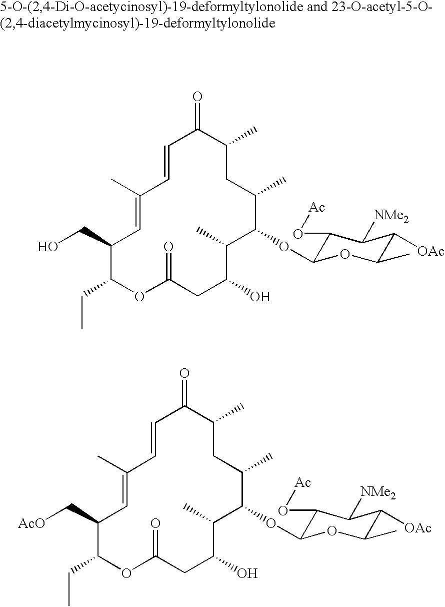

A mixture of acetic anhydride (0.70 mL) and l9-deformyl-5-O-mycinosyltylonolide (1.33 g) in acetone (25 mL) was stirred for 2 hours and concentrated under reduced pressure. The residue was dissolved in methylene chloride, washed with saturated sodium bicarbonate solution, dried with magnesium sulfate and concentrated to give a clear oil which was purified by flash chromatography (hexanes/acetone/triethylamine) to give 5-O-(2,4-diacetylmycinosyl)-19-deformyltylonolide (0.80 g) and 23-O-acetyl-5-O-(2,4-diacetylmycinosyl)-19-deformyltylonolide (0.267 g)

### EXAMPLE 44

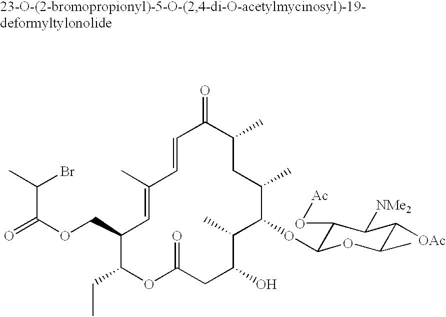

2-Bromopropionyl bromide (0.024 mL) was added to a solution of 5-O-(2,4-diacetyl-mycinosyl)-19-deformyltylonolide (0. 10 g) and pyridine (0. 05 mL) in methylene chloride (2 mL) at 0° C. and stirred until the starting material was consumed as judged by TLC analysis. The reaction mixture was diluted with methylene chloride (50 mL), washed with saturated sodium bicarbonate solution (10 mL), dried with magnesium sulfate and concentrated to give the desired product (139 mg) which was used without further purification.

### EXAMPLE 45

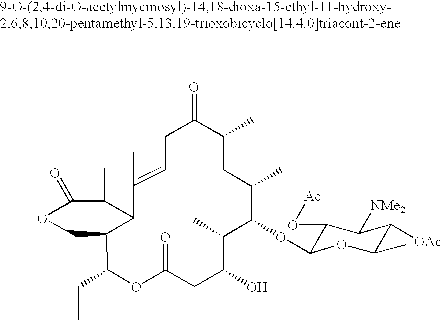

Tributyltin hydride (0.053 mL) and 1,1′-azobis(cyclohexanecarbonitrile) (5 mg) were added to a solution of crude 23-O-(2-bromopropionyl)-5-O-(2,4-diacetylmycinosyl)-19-deformyltylonolide (140 mg) in benzene. The reaction mixture was maintained at reflux for 3.25 h, allowed to cool to room temperature, diluted with acetonitrile, and extracted with hexanes (5×10 mL). The acetonitrile layer was concentrated and the residue (0.117 g) was purified by flash chromatography (hexanes/acetone/triethylamine) to give the cyclic product.

### EXAMPLE 46

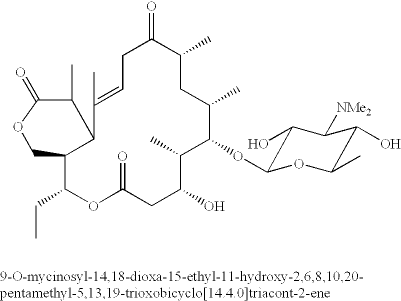

The 9-O-(2,4-diacetylmycinosyl)-14,18-dioxa-15-ethyl-11-hydroxy-2,6,8,10,20-pentamethyl-5,13,19-trioxobicyclo[14.4.0]triacont-2-ene from the previous example was dissolved in methanol and maintained at 40° C. Overnight,then concentrated under reduced pressure to give the desired product.

### EXAMPLE 47

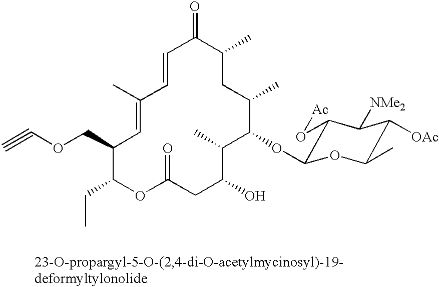

A suspension of 35% potassium hydride in oil (180 mg) was placed under inert atmosphere and washed twice with 5 mL portions of dry hexane, then suspended in 10 mL of dry tetrahydrofuran at 0° C. Propargyl bromide (80% in toluene, 0.10 mL) was added and stirred for 1 minute. A solution of 5-O-(2,4-diacetylmycinosyl)-19-deformyltylonolide (500 mg) in DMSO (5 mL) was added to the suspension and stirred for 1 minute, followed by addition of more propargyl bromide (0.8 mL). The reaction mixture was stirred at 0° C. for 3 hours, then poured into ethyl acetate, washed repeatedly with water, dried with magnesium sulfate, and concentrated. The residue was flash chromatographed (hexane/acetone/triethylamine) to give the desired propargyl ether (207 mg).

### EXAMPLE 48

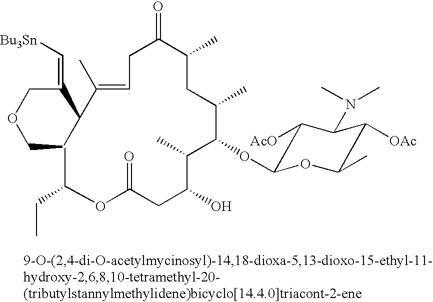

Tributyltin hydride (0.115 mL) and azobis(cyclohexanecarbonitrile) (10 mg) were added to a hot solution of 23-O-propargyl-5-O-(2,4-diacetylmycinosyl)-19-deformyltylonolide (100 mg) in 10 mL of toluene under an inert atmosphere and maintained at reflux for 2 hours. Additional tributyltin hydride (0.10 mL) was added, and reflux was continued for an additional 12 hours. The solvent was removed under reduced pressure and the residue was dissolved in acetonitrile and hexane. The acetonitrile layer was washed repeatedly with hexane and concentrated. The residue was flash chromatographed (hexane/acetone with 1% triethylamine) to give the cyclized product (58 mg).

### EXAMPLE 49

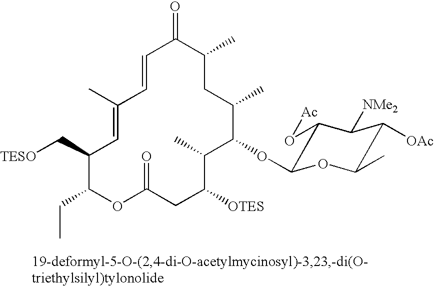

A solution of 19-deformyl-5-O-(2,4-diacetylmycinosyl)tylonolide (200 mg) in pyridine (2 mL) was treated with chlorotriethylsilane (0.58 mL) at 60° C. for 2 hours. The reaction was cooled to ambient temperature, quenched with sat. NaHCO₃, and extracted with CHCl₃. The extract was washed with brine, dried over Na₂SO₄, filtered, and evaporated. The product was isolated by silica gel chromatography (ethyl acetate/hexanes+1% Et₃N). ¹³C-NMR (CDCl₃): δ 204, 171.7, 169.8, 169.2, 147.7, 142.6, 134.0, 118.6, 100.9, 75.2, 71.7, 70.8, 70.5, 62.5, 47.4, 44.4, 42.3, 41.2 (2C), 33.7, 25.2, 21.4, 21.2, 17.9, 17.4, 17.3, 12.8, 9.7, 7.0 (3C), 6.7 (3C), 5.1 (3C), 4.3 (3C).

### EXAMPLE 50

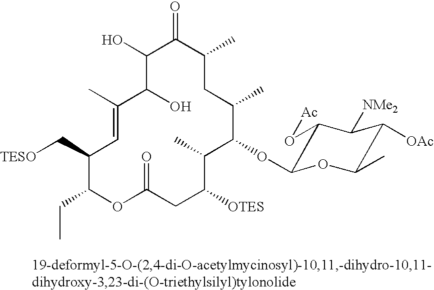

A solution of 19-deformyl-5-O-(2,4-di-O-acetylmycinosyl)-2,23-di(O-triethylsilyl)tylonolide (140 mg) in 1 mL of CH₂Cl₂ was cooled to −78° C. under inert atmosphere and treated with tetramethylethylenediamine (0.03 mL) followed by a solution of osmium tetraoxide (44 mg) in 0.3 mL of CH₂Cl₂. The brown-black solution was allowed to warm to 10° C. over 2.75 hours, and quenched by addition of 1 mL of tetrahydrofuran and 2 mL of sat. NaHSO₃. This mixture was brought to reflux and kept for 2 hours, then cooled to ambient temperature and kept 12 hours. The mixture was partitioned between ethyl acetate and sat. NaHCO₃, and the organic extract was washed with brine, dried over Na₂SO₄, filtered, and concentrated to a brown oil. The product was purified by silica gel chromatography (acetone/hexanes+1% Et₃N). ¹³C-NMR (CDCl₃): δ 214.3, 171.3, 169.8, 169.3, 139.3, 126.2, 100.4, 79.8, 76.3, 76.1, 71.7, 70.7, 70.5, 69.7, 67.2, 63.4, 46.1, 43.5, 42.8, 40.7, 41.3 (2C), 38.3, 35.4, 25.3, 21.3, 21.2, 18.3, 18.1, 17.6, 17.4, 14.1, 11.6, 11.4, 9.8, 9.6, 9.5, 6.9 (3C), 6.7 (3C), 5.0 (3C), 4.3 (3C).

### EXAMPLE 51

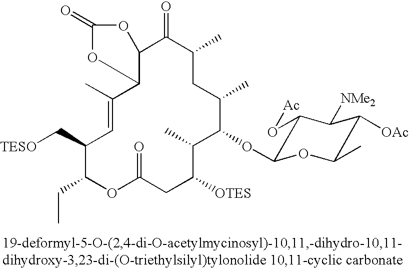

A solution of 19-deformyl-5-O-(2,4-di-O-acetylmycinosyl)-10,11-dihydro-10,11-dihydroxy-3,23-di(O-triethylsilyl)tylonolide (40 mg) in 0.35 mL of pyridine was treated with a 20% solution of phosgene in toluene (0.046 mL) for 16 hours at ambient temperature. The mixture was partitioned between ethyl acetate and sat. NaHCO₃, and the organic extract was washed with brine, dried over Na₂SO₄, filtered, and concentrated to a brown oil. The product was purified by silica gel chromatography (acetone/hexanes+1% Et₃N). ¹³C-NMR (CDCl₃): δ 206.8, 171.1, 169.8, 169.2, 153.3, 133.2, 129.9, 101.0, 84.0, 82.0, 80.3, 75.6, 71.6, 70.9, 70.4, 69.2, 67.2, 63.1, 46.2, 44.2, 43.7, 43.1, 41.2, 40.6, 35.0, 32.6, 25.6, 21.3, 21.2, 18.4, 17.7, 17.4, 11.5, 10.6, 9.7, 9.0, 7.0 (3C), 6.7 (3C), 5.2 (3C), 4.3 (3C).

### EXAMPLE 52

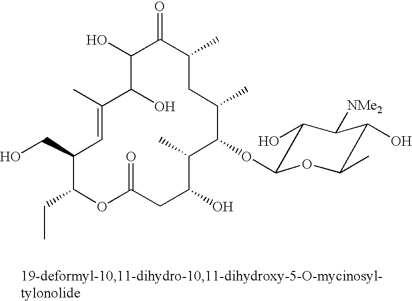

A solution of 19-deformyl-5-O-(2,4-di-O-acetylmycinosyl)-10,11-dihydro-10,11-dihydroxy-3,23-di-(O-triethylsilyl)tylonolide (21 mg) in 0.1 mL of tetrahydrofuran is treated with 0.14 mL of 1 M tetrabutylammonium fluoride in tetrahydrofuran for 16 hours. The mixture is concentrated, and the residue is dissolved in methanol (1 mL) and kept for 12 hours at 50° C. The mixture is concentrated, and the residue is partitioned between ethyl acetate and sat. NaHCO₃. The organic extract is washed with brine, dried over sodium sulfate, filtered, and evaporated.

### EXAMPLE 53

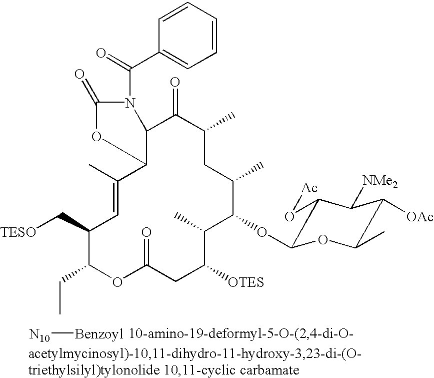

A solution of 19-deformyl-5-O-(2,4-di-O-acetylmycinosyl)-10,11-dihydro-10,11-dihydroxy-3,23-di(O-triethylsilyl)tylonolide (943 mg) and dibutyltin oxide (500 mg) in 15 mL of dichloroethane are heated at reflux for 4 hours, using a Dean-Stark trap to remove water. Benzoyl isothiocyanate (0.23 mL) and triethylamine (0.17 mL) are added, and reflux was continued for an additional 1 hour. Tetrabutylammonium bromide (332 mg) is added, and heating is continued for another 2 hours. The reaction mixture is cooled and partitioned between ethyl acetate and water, and the organic phase is washed with brine, dried over sodium sulfate, filtered, and concentrated. The product is isolated by silica gel chromatography.

### EXAMPLE 54

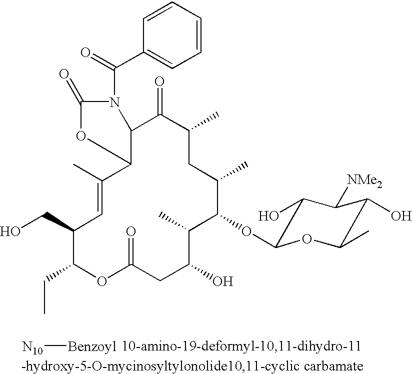

A solution of N₁₀-Benzoyl 10-amino-19-deformyl-5-O-(2,4-di-O-acetylmycinosyl)-10,11-dihydro-11-hydroxy-3,23-di-(O-triethylsilyl)tylonolide 10,11-cyclic carbamate (24 mg) in 0.1 mL of tetrahydrofuran is treated with 0.14 mL of 1 M tetrabutylammonium fluoride in tetrahydrofuran for 16 hours. The mixture is concentrated, and the residue is dissolved in methanol (1 mL) and kept for 12 hours at ambient temperature. The mixture is concentrated, and the residue is partitioned between ethyl acetate and sat. NaHCO₃. The organic extract is washed with brine, dried over sodium sulfate, filtered, and evaporated.

### EXAMPLE 55

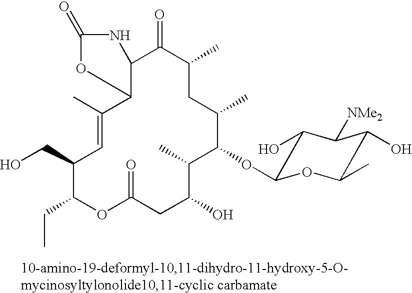

A solution of N₁₀-Benzoyl 10-amino-19-deformyl-10,11-dihydro-11-hydroxy-5-O-mycinosyltylonolide 10,11-cyclic carbamate (75 mg) in 1 mL of methanol is treated with cesium carbonate (30 mg) for 2 hours at ambient temperature. The mixture is concentrated, and the residue is partitioned between water and ethyl acetate. The organic phase is washed with brine, dried over sodium sulfate, and concentrated. The product is purified by silica gel chromatography.

### EXAMPLE 56

MIC Testing

The MIC was determined by the tube broth dilution method with cation-adjusted Mueller-Hinton broth (CAMHB) for S. aureus strains, or with CAMHB supplemented with 2% lysed horse blood for S. pneumoniae strains, according to the procedures recommended by National Committee for Clinical Laboratory Standards (NCCLS) (1). See National Committee for Clinical Laboratory Standards. 2000; Methods for Dilution Antimicrobial Susceptibility Tests for Bacterial That Grow Aerobically: Approved Standard-Fifth Edition. M7-A5. National Committee for Clinical Laboratory Standards, Wayne, Pa.; and Quality Control Guidelines for Disk Diffusion and Broth Microdilution Antimicrobial Susceptibility Tests with Seven Drugs for Veterinary Applications. J Clin Microbiol. January 2000; 38(l):453-5, which are each incorporated herein by reference.

Streptococcus pneumoniae and Staphylococcus aureus strains, including both erythromycin-susceptible and erythromycin-resistance strains, were obtained from ATCC. S. pneumoniae strains were grown on blood agar base supplemented with 5% defibrinated sheep blood (Teknova, Calif.). S. aureus ATCC 33591 and 14154 were grown on nutrient agar, whereas S. aureus ATCC 6538p and 29213 were grown on micrococcus agar and trypticase soy agar respectively.

Serial twofold dilutions were prepared in CAMHB or CAMHB supplemented with 2% lysed horse blood in tubes (1 ml/tube). The twofold dilutions of antibiotics used are 50, 25, 12.5, 6.25, 3.125, 1.56, 0.78, 0.39, 0.19, 0.098, 0.049 and 0 ug/ml. The bacterial inocula were prepared in CAMHB by directly suspending colonies grown on an appropriate 18-24-hour agar plate. The suspensions were adjusted to match the 0.5 McFarland standard (1×10⁸ CFU/ml) and inoculated to tubes containing serial antibiotic dilutions to make a final concentration of 5×10⁵ CFU/ml. The tubes were then incubated at 35° C. for 16 to 20 hours in an ambient air incubator and the MICs were determined. The quality control strains, S. pneumoniae ATCC 49619 and S. aureus ATCC 29213 were included in the tests.

What is claimed is:

1. A sixteen-membered macrolide
having a side chain Z at C-8 of the macrolide wherein Z is selected from the group consisting of: hydrogen, —O—(CH₂)ₙ-cycloalkyl; —O—(CH₂)ₙ-heterocyclo; —O—(CH₂)ₙ-aryl; —O—(CH₂)ₙ—CH═CH-aryl; and —O—(CH₂)ₙ—CH═CH—CH₂-aryl where n is 0-5;
or a fused bicyclic compound
where one of the cyclic components is a sixteen-membered macrolide and the other is a five- or six-membered heterocyclic moiety.

2. The macrolide as in claim 1 of the formula 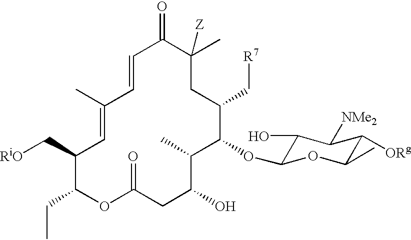
where Rⁱ is hydrogen, or mycinose;
R^{g} is hydrogen, mycarose, 4-acyl-mycarose, or 4-sulfonyl-mycarose;
R⁷ is hydrogen, methyl, hydroxymethyl, aminomethyl or CHO; and,
Z is selected from the group consisting of. —O—(CH₂)ₙ-cycloalkyl; —O—(CH₂)ₙ-heterocyclo; —O—(CH₂)ₙ-aryl; —O—(CH₂)ₙ—CH═CH-aryl; and —O—(CH₂)ₙ—CH═CH—CH₂-aryl where n is 0-5.

3. The macrolide as in claim 2 wherein the 4-acyl or 4-sulfonyl group in mycarose is selected from the group consisting of isovaleryl; phenylacetyl; phenylthioacetyl; phenylsulfonylacetyl; 4-nitrophenylacetyl; 4-nitrophenylsulfonyl; and phenylethanesulfonyl.

4. The macrolide as in claim 2 wherein the aryl is selected from the group consisting of

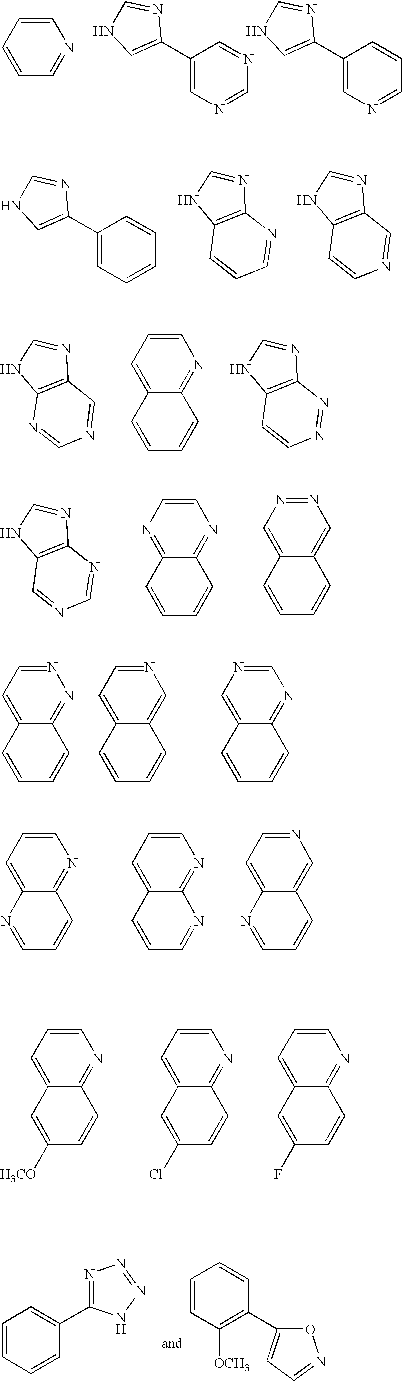

5. The bicyclic compound of claim 1 of the formula 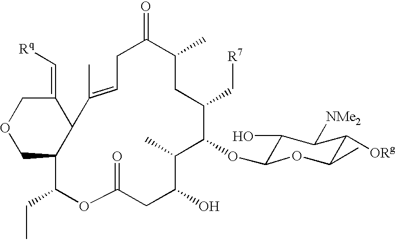
wherein
R⁷ is hydrogen, methyl, hydroxymethyl, aminomethyl or CHO; and,
R^{g} is hydrogen, mycarose or 4-acyl-mycarose; and,
R^{q} is C₁-C₅ alkyl, aryl, —(CH₂)ₙ.-cycloalkyl; —(CH₂)ₙ-heterocyclo; —(CH₂)ₙ-aryl; —(CH₂)ₙ—CH═CH-aryl; —(CH₂)ₙ—CH═CH—CH₂-aryl; and, —(CH₂)n-NHC(=O)—(CH₂)ₘ-aryl where n and m are each independently 0-5.

6. The bicyclic compound of claim 1 of the formula 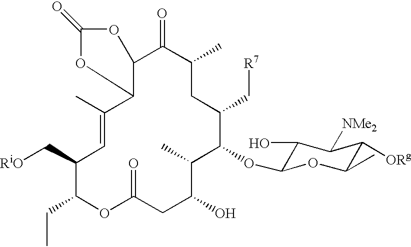
wherein
Rⁱ is hydrogen or mycinose;
R^{g} is hydrogen, mycarose or 4-acyl-mycarose; and,
R⁷ is hydrogen, methyl, hydroxymethyl, aminomethyl or CHO.

7. The bicyclic compound of claim 1 of the formula 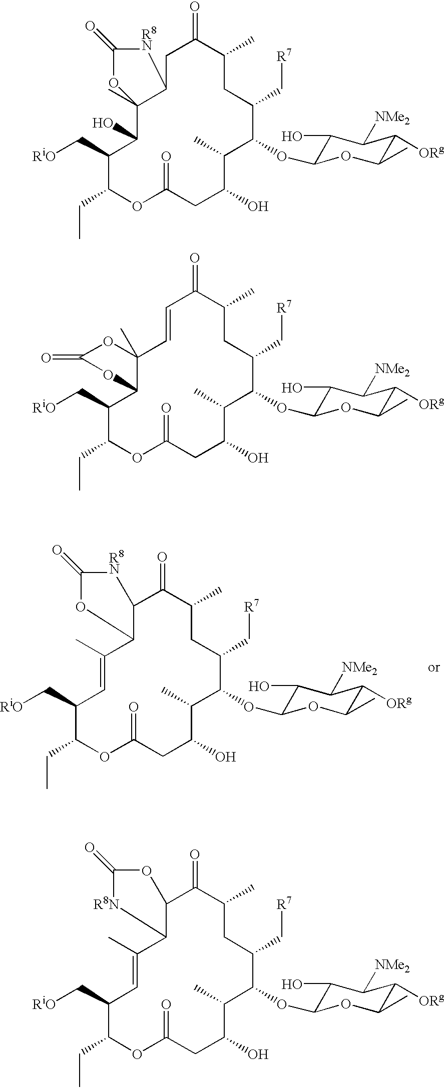
wherein
Rⁱ is hydrogen or mycinose;
R^{g} is hydrogen, mycarose or 4-acyl-mycarose;
R⁷ is hydrogen, methyl, hydroxymethyl, aminomethyl or CHO; and,
R⁸ is hydrogen, C₁-C₅ alkyl, aryl, —(CH₂)ₙ-cycloalkyl; —(CH₂)ₙ-heterocyclo; (CH₂)ₙ aryl; (CH₂)ₙ CH—CH aryl; (CH₂)ₙ CH—CH CH₂ aryl; and, (CH₂)ₙ NHC(═O)—(CH₂)ₘ-aryl where n and m are each independently 0-5.

8. The bicyclic compound of claim 1 of the formula 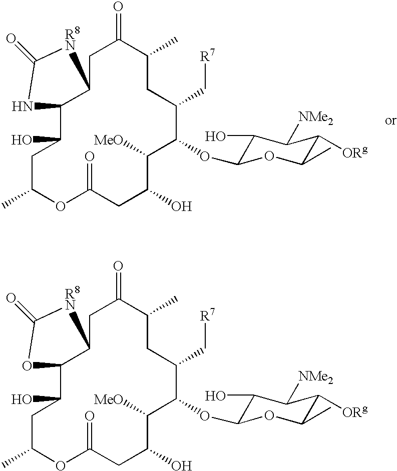
wherein
R^{g} is hydrogen, mycarose or 4-acyl-mycarose;
R⁷ is hydrogen, methyl, hydroxymethyl, aminomethyl or CHO; and,
R⁸ is hydrogen, C₁-C₅ alkyl, aryl, —(CH₂)ₙ-cycloalkyl; —(CH₂)ₙ-heterocyclo; —(CH₂)ₙ-aryl; —(CH₂)ₙ—CH═CH-aryl; —(CH₂)ₙ—CH═CH—CH₂-aryl; —(CH₂)n-NHC(═O)—(CH₂)ₘ-aryl where n and m are each independently 0-5.

9. The compound as in claim 5, 6, 7, or 8 wherein the aryl is selected from the group consisting of

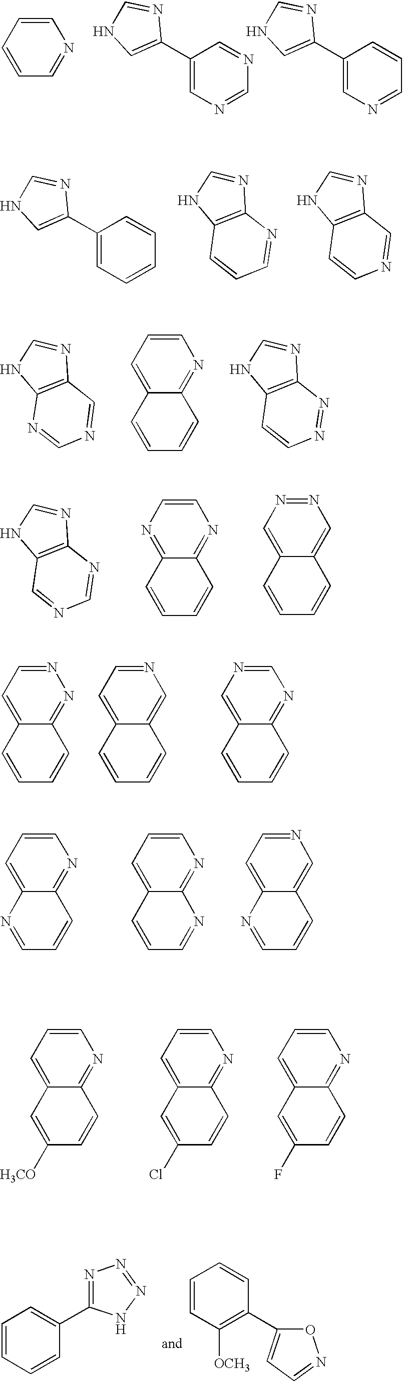

10. The compound as in claim 5, 6, 7, or 8 wherein the 4-acyl or 4-sulfonyl group in mycarose is selected from the group consisting of isovaleryl; phenylacetyl; phenylthioacetyl; phenylsulfonylacetyl; 4-nitrophenylacetyl; 4-nitrophenylsulfonyl; and phenylethanesulfonyl.

11. A method of making a 8-hydroxy-9-oxo macrolide comprising converting a 9-oxo macrolide into a 8,9-silyl enolether and oxidizing the 8,9-silyl enolether to the 8-hydroxy-9-oxo macrolide.

12. A method of making a bicyclic compound wherein one of the cyclic components is a sixteen-membered macrolide and the other is a six-membered cyclic ether comprising
converting a 12-ene-14-hydroxymethyl-macrolide to a 12-ene-14-alkynylalkoxymethyl macrolide; and
treating the 12-ene-14-alkynylalkoxymethyl macrolide with a trialkyl-tin-hydride.

13. A dihydroxylation method comprising
hydroxylating a 10-ene sixteen-membered macrolide to form a 10,11-dihydroxy macrolide.

14. A method of adding a nucleophile at C-12 of a 9-oxo-10-ene-12,13-epoxy sixteen-membered macrolide comprising
treating the macrolide with a transition metal catalyst in the presence of the nucleophile.

15. A method of making a 9-oxo-10-ene-12-hydroxy sixteen-membered macrolide comprising
treating a 9-oxo-11-ene-13-hydroxy macrolide with an epoxidizing agent to form a 11, 12-epoxide; and
treating the epoxide with a base to make the 9-oxo-10-ene-12-hydroxy sixteen-membered macrolide.
